(19) United States
(12) Patent Application Publication
(10) Pub. No.: US 2008/0249042 A1
(43) Pub. Date: Oct. 9, 2008

(54) All-Trans-Retinol: All-Trans-13,14-Dihydroretinol Saturase and Methods of Its Use

(75) Inventors: Alexander R. Moise, Cleveland, OH (US); Vladimir A. Kuksa, Seattle, WA (US); Krzysztof Palczewski, Bay Village, OH (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: UNIVERSITY OF WASHINGTON, Seattle, WA (US)

(21) Appl. No.: 11/574,976

(22) PCT Filed: Sep. 9, 2005
(86) PCT No.: PCT/US05/32462

§ 371 Date: Jun. 21, 2007

Related U.S. Application Data
(60) Provisional application No. 60/609,038, filed on Sep. 9, 2004.

(51) Int. Cl. A61K 31/7088; C12P 7/02; C12N 9/00; C07H 21/00; C12N 15/00; C12N 5/00; C12N 1/00; C12P 19/34; C07K 16/00; G01N 33/53; A61K 31/07

(52) U.S. Cl. 514/44; 435/155; 435/183; 536/23.2; 435/320.1; 435/325; 435/243; 435/69.1; 530/387.1; 530/388.1; 530/389.1; 435/7.2; 514/725

(57) ABSTRACT

Compositions of all-trans-retinol: all-trans-13,14-dihydroretinal saturase and methods of use thereof are provided.

### CROSS-REFERENCE TO RELATED APPLICATIONS

This application claims the benefit of U.S. Application Ser. No. 60/609,038, filed Sep. 9, 2004, the entire disclosures of which is incorporated herein by reference.

### STATEMENT OF GOVERNMENT SUPPORT

This invention was made with Government support by Grant Nos. R03 EY0 15399-01, EY08061, and EY08123 awarded by the National Institutes of Health. The Government has certain rights in this invention.

### FIELD

The invention generally relates to compositions of all-trans-retinol: all-trans-13,14-dihydroretinol saturase, enzymatic products, and methods of use thereof.

### BACKGROUND

Retinoids are essential for many important biological functions, such as development, immunity, cellular differentiation, and vision of vertebrates. Retinoids encompassing both natural derivatives of all-trans-retinol and their synthetic analogues exert their functions through several active compounds. Esterification of retinol by lecithin-retinol acyltransferase (LRAT) leads to retinyl esters, which represent both a major storage form of vitamin A and an intermediate of the visual cycle. Ruiz, et al. J Biol Chem 274:3834-3841, 1999; Batten, et al. J Biol Chem 279:10422-10432, 2004; Imanishi, et al. J Cell Biol 164:373-383, 2004. In retinal pigment epithelium (RPE) an unidentified enzyme carries out the isomerization of all-trans-retinol either directly or through an ester intermediate to generate 11-cis-retinol, which can be oxidized to 11-cis-retinal, the visual chromophore. Kuksa, et al. Vision Res 43:2959-2981, 2003. Reversible oxidation to retinal can be carried out by several members of the microsomal, short-chain alcohol dehydrogenase family (SCAD) and possibly by class I, II, and IV medium-chain alcohol dehydrogenases (ADH). Chou, et al. J Biol Chem 277:25209-25216, 2002; Duester, et al. Chem Biol Interact 143-144, 201-210, 2003. Oxidation of retinal by retinal dehydrogenase (RALDH) types 1, 2, 3 and 4 generates retinoic acid (RA), which controls development and cellular differentiation via nuclear receptors. Bhat, et al. Gene 166:303-306, 1995; Penzes, et al. Gene 191:167-172, 1997; Wang, et al. J Biol Chem 271:16288-16293, 1996; Zhao, et al. Eur J Biochem 240:15-22, 1996; Mic, et al. Mech Dev 97:227-230, 2000; Lin, et al. J Biol Chem 278:9856-9861, 2003; Chambon, Faseb J 10:940-954, 1996. RA-inducible cytochrome P450 enzymes CYP26A1 and B1 carry out the catabolism of RA to polar 4-hydroxy-RA, 4-oxo-RA and 18-hydroxy-RA. Abu-Abed, et al. J Biol Chem 273:2409-2415, 1998; Fujii, et al. Embo J 16:4163-4173, 1997; White, et al. J Biol Chem 271:29922-29927, 1996; White, et al. Proc Natl Acad Sci USA 97:6403-6408, 2000. Specific localization of RA anabolizing and catabolizing enzymes are essential for embryonic patterning. Other pathways generate retro-retinoids such as 14-hydroxy-4,14-retro-retinol (14-HRR) and anhydroretinol (AR), whose opposing effects control cell growth. Buck, et al. Science 254:1654-1656, 1991; Buck, et al. J Exp Med 178:675-680, 1993. Given the low levels and labile nature of retinoids in biological systems, and the incompletely understood mechanism of their biotransformations, a need exists in the art to identify many of the enzymes involved in retinoid metabolism.

### SUMMARY

The invention is generally related to compositions of all-trans-retinol: all-trans-13,14-dihydroretinol saturase and methods of use thereof. An isolated polypeptide is provided comprising the contiguous sequence of human, mouse or monkey all-trans-retinol: all-trans-13,14-dihydroretinol saturase, or a functionally active fragment thereof. In a further aspect, the isolated polypeptide comprises the contiguous sequence of human all-trans-retinol: all-trans-13,14-dihydroretinol saturase (GenBank Accession Number gi46329587). An isolated polynucleotide is provided comprising the contiguous sequence of human, mouse or monkey all-trans-retinol: all-trans-13,14-dihydroretinol saturase, or a functionally active fragment thereof.

A method for treating a disease state in a mammalian subject comprises administering to the mammalian subject a compound that activates all-trans-retinol: all-trans-13,14-dihydroretinol saturase activity in the mammalian subject. A method for treating a disease state in a mammalian subject comprises administering to the mammalian subject a compound that inhibits all-trans-retinol: all-trans-13,14-dihydroretinol saturase activity in the mammalian subject. In a further aspect, a method for treating for treating a disease state in a mammalian subject comprises administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier. The disease state includes, but is not limited to, retinal disease, blindness, autoimmune disease, cancer, neoplastic disease, or a skin condition or disorder.

A method of producing all-trans-(13,14)-dihydroretinol is provided comprising expressing a heterologous nucleic acid which hybridizes under stringent conditions comprising hybridization in aqueous solution containing 4-6×SSC at 65-68° C., or 42° C. in 50% fornamide, to a polynucleotide that codes for human RetSat (GenBank Accession Number gi46329587), mouse RetSat (GenBank Accession Number AY704159) or monkey (macaque) RetSat (GenBank Accession Number AY707524), or a functionally active fragment thereof, in a host cell. In one aspect, the host cell is a mammalian host cell

An isolated polypeptide is provided comprising the contiguous sequence of human, mouse or monkey all-trans-retinol: all-trans-13,14-dihydroretinol saturase, or a functionally active fragment thereof. In a further aspect, the isolated polypeptide comprises the contiguous sequence of human all-trans-retinol: all-trans-13,14-dihydroretinol saturase (GenBank Accession Number gi46329587).

An isolated polynucleotide is provided comprising the contiguous sequence of human, mouse or monkey all-trans-retinol: all-trans-13,14-dihydroretinol saturase, or a functionally active fragment thereof.

An expression construct is provided In a detailed aspect, the transcriptional promoter is a heterologous promoter. A cultured prokaryotic or eukaryotic cell is provided which is transformed or transfected with the expression construct. In a further aspect, the eukaryotic cell is a mammalian cell.

A vector is provided comprising the expression construct which comprises the following operably linked elements: a transcriptional promoter; a RETSAT polynucleotide which hybridizes under stringent conditions comprising hybridization in aqueous solution containing 4-6×SSC at 65-68°, or 42° C. in 50% formamide, to a polynucleotide encoding human RetSat (GenBank Accession Number gi46329587), mouse RetSat (GenBank Accession Number AY704159) and monkey (macaque) RetSat (GenBank Accession Number AY707524) or the full length complement of the polynucleotide, wherein the RetSat polypeptide comprises the contiguous amino acid sequence of the human, mouse or monkey polypeptide or a functionally active fragment thereof; and a transcriptional terminator. In a further aspect an isolated host cell comprises the vector. A method for producing a RetSat polypeptide is provided which comprises growing cells transformed or transfected with the vector, and isolating the RetSat polypeptide from the cells. In a detailed aspect, the cells are bacterial cells or mammalian cells.

An antibody is provided that binds to human RetSat polypeptide. In a further aspect, the antibody is a monoclonal antibody, a polyclonal antibody, a single chain antibody, a heavy chain antibody, an F(ab′)2, F(ab′), or Fv fragment.

A method of identifying agonists or antagonists of a eukaryotic Retsat polypeptide comprising: administering a candidate compound to a first cell that expresses a Retsat polypeptide, and determining whether the candidate compound produces a physiological change by the first cell.

A pharmaceutical composition is provided comprising all-trans-13,14-dihydroretinol, all-trans-13,14-dihydroretinoic acid and/or all-trans-13,14-dihydroretinoid derivative, and a pharmaceutically acceptable carrier. The pharmaceutical composition can be formulated, for example, for topical administration, oral administration, intravenous administration, intraocular injection or perioccular injection. In a further aspect the pharmaceutical composition can be the all-trans 13,14-dihydroretinoid derivative is a retinyl ester.

A method for treating for treating retinal disease or blindness in a mammalian subject is provided comprising administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

A method for treating a retinal disease state or blindness in a mammalian subject is provided comprising administering to the mammalian subject a compound that activates all-trans-retinol: all-trans-13,14-dihydroretinol saturase activity in the mammalian subject.

A method for treating for treating autoimmune disease in a mammalian subject comprising administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

A method for treating an autoimmune disease in a mammalian subject is provided comprising administering to the mammalian subject a compound that activates all-trans-retinol: all-trans-13,14-dihydroretinol saturase activity in the mammalian subject.

A method for treating for treating a skin condition or disorder in a mammalian subject is provided comprising administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

A method for treating a skin condition or disorder in a mammalian subject is provided comprising administering to the mammalian subject a compound that activates all-trans-retinol: all-trans-13,14-dihydroretinol saturase activity in the mammalian subject.

A method for treating a neoplastic disease state in a mammalian subject is provided comprising administering to the mammalian subject a compound that inhibits all-trans-retinol: all-trans-13,14-dihydroretinol saturase activity in a neoplastic cell.

### BRIEF DESCRIPTION OF THE DRAWINGS

FIG. 1 shows the identification of vertebrate proteins with similarity to plant and cyanobacteria CRTISO.

FIG. 2 shows the subcellular localization of mouse RetSat in transfected cells.

FIG. 3 shows enzyme activities of tomato CRTISO and mouse RetSat in transfected cells.

FIG. 4 shows the identification of the biosynthetic product of the conversion of all-trans-retinol by mouse RetSat.

FIG. 5 shows the isomeric form of the substrate of mouse RetSat.

FIG. 6 shows RetSat activity towards all-trans-retinal.

FIG. 7 shows RetSat activity towards all-trans-retinoic acid.

FIG. 8 shows RetSat activity in homogenized cells.

FIG. 9 shows the identification of all-trans-13,14-dihydroretinol in various tissues.

FIG. 10 shows LRAT activity.

FIG. 11 shows the analysis of metabolism of all-trans-ROL palmitate in the liver of Lrat−/− mice.

FIG. 12 shows the oxidation of all-trans-ROL and all-trans-DROL to the respective aldehyde.

FIG. 13 shows the oxidation of all-trans-RAL and all-trans-DRAL to all-trans-RA and all-trans-DRA, respectively.

FIG. 14 shows the oxidation of all-trans-RA and all-trans-DRA.

FIG. 15 shows the response of F9-RARE-lacZ reporter cell line to RA and DRA.

FIG. 16 shows the activation of DR1 elements by all-trans-DRA, all-trans-RA, and 9-cis-RA.

FIG. 17 shows compound all-trans-4-oxo-DRA (VI) was characterized by [1H]-NMR.

FIG. 18 shows analysis of metabolism of all-trans-DROL in the liver of Lrat−/− mice.

FIG. 19 shows analysis of metabolism of all-trans-RA in the liver of Lrat−/− mice.

FIG. 20 shows conversion of all-trans-DROL into all-trans-DRA by RPE microsomes.

FIG. 21 shows the reaction catalyzed by plant and cyanobacterial CRTISO.

FIG. 22 shows the synthesis of all-trans-13,14-dihydroretinol.

FIG. 23 shows the reaction catalyzed by RetSat converting all-trans-retinol into all-trans-13,14-dihydroretinol.

FIG. 24 shows the metabolism of all-trans-ROL and all-trans-DROL.

### DETAILED DESCRIPTION

Retinoids carry out essential functions in vertebrate development and vision. Many of the retinoid processing enzymes remain to be identified at the molecular level. To expand the knowledge of retinoid biochemistry in vertebrates we studied the enzymes involved in plant metabolism of carotenoids, a related group of compounds. We identified a family of vertebrate enzymes that share significant similarity and a putative phytoene dehydrogenase domain with a recently described plant carotenoid isomerase, CRTISO, which isomerizes prolycopene to all-trans-lycopene. Comparison of heterologously-expressed mouse and plant enzymes indicates that unlike plant CRTISO, the CRTISO-related mouse enzyme is inactive towards prolycopene. Instead, the CRTISO-related mouse enzyme is a retinol saturase carrying out the saturation of the 13-14 double bond of all-trans-retinol to produce all-trans-13,14-dihydroretinol. The product of mouse retinol saturase (RetSat) has a shifted UV absorbance maximum, λₘₐₓ=290 nm, compared to the parent compound, all-trans-retinol (λₘₐₓ=325 nm), and its MS analysis (m/z=288) indicates saturation of a double bond. The product was further identified as all-trans-13,14-dihydroretinol as its characteristics matched those of a synthetic standard. Mouse RetSat is membrane associated and expressed in many tissues, with the highest levels in liver, kidney, and intestine. All-trans-13,14-dihydroretinol was also detected in several tissues of animals maintained on a normal diet. Thus, saturation of all-trans-retinol to all-trans-13,14-dihydroretinol by RetSat produces a new metabolite of yet unknown biological function.

The metabolism of vitamin A is a highly regulated process that generates essential mediators involved in the development, cellular differentiation, immunity, and vision of vertebrates. Retinol saturase converts all-trans-retinol to all-trans-13,14-dihydroretinol. The present study demonstrates that the enzymes involved in oxidation of retinol to retinoic acid and then to oxidized retinoic acid metabolites are also involved in the synthesis and oxidation of all-trans-13,14-dihydroretinoic acid. All-trans-13,14-dihydroretinoic acid can activate retinoic acid receptor/retinoid X receptor heterodimers but not retinoid X receptor homodimers in reporter cell assays. All-trans-13,14-dihydroretinoic acid was detected in vivo in Lrat−/− mice supplemented with retinyl palmitate. Thus, all-trans-13,14-dihydroretinoic acid is a naturally occurring retinoid and a potential ligand for nuclear receptors. This new metabolite can also be an intermediate in a retinol degradation pathway or it can serve as a precursor for the synthesis of bioactive 13,14-dihydroretinoid metabolites.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

The term “RetSat locus” and “RETSAT gene” refer to the coding sequences, intervening sequences and regulatory elements controlling transcription and/or translation. The terms “RETSAT locus” and “RETSAT gene” include all allelic variations of RETSAT. In exemplary embodiments, the RETSAT gene is GenBank Accession Number gi46329587 for human RetSat, GenBank Accession Number AY704159 for mouse RetSat and GenBank Accession Number AY707524 for monkey (macaque) RetSat, the disclosures of which are incorporated by reference herein.

The term “RETSAT nucleic acids” refers to polynucleotides from the RetSat locus, such as those encoding RetSat polypeptides, including mRNAs, DNAs, cDNAs, genomic DNA, as well as antisense nucleic acids, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and typically 80%, more typically 90% sequence identity, to the RETSAT nucleic acid of human RetSat (GenBank Accession Number gi46329587), mouse RetSat (GenBank Accession Number AY704159) and monkey (macaque) RetSat (GenBank Accession Number AY707524).

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides or larger. Polynucleotides and nucleic acids include RNA, cDNA, genomic DNA, synthetic forms, and mixed polymers, both sense and antisense strands, and can also be chemically or biochemically modified or can contain non-natural or derivatized nucleotide bases, as will be readily appreciated by the skilled artisan. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “oligonucleotide” refers to a polynucleotide of from about six (6) to about one hundred (100) nucleotides or more in length. Thus, oligonucleotides are a subset of polynucleotides. Oligonucleotides can be synthesized on an automated oligonucleotide synthesizer (for example, those manufactured by Applied BioSystems (Foster City, Calif.)) according to specifications provided by the manufacturer.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

“Retsat polypeptide” refers to a polypeptide encoded by a RETSAT gene, and fragments, derivatives or analogs thereof. The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide typically having at least 10 contiguous amino acids, more typically at least 20, still more typically at least 50 contiguous amino acids of the Retsat polypeptide. A derivative is a polypeptide having conservative amino acid substitutions, as compared with another sequence. Derivatives further include, for example, glycosylations, acetylations, phosphorylations, and the like. An analog of a “polypeptide” can be, for example, a polypeptide containing one or more analogs of an amino acid (e.g., unnatural amino acids, and the like), polypeptides with substituted linkages as well as other modifications known in the art, both naturally and non-naturally occurring. Ordinarily, such polypeptides will be at least about 50% identical to the native Retsat amino acid sequence, typically in excess of about 90%, and more typically at least about 95% identical.

The terms “amino acid” or “amino acid residue”, as used herein, refer to naturally occurring L amino acids or to D amino acids as described further below. The commonly used one- and three-letter abbreviations for amino acids are used herein (see, e.g., Alberts et al., Molecular Biology of the Cell, 3d ed., Garland Publishing, Inc., New York, 1994).

The term “heterologous” refers to a nucleic acid or polypeptide from a different source, (e.g., a tissue, organism or species), as compared with another nucleic acid or polypeptide.

The term “isolated” refers to a nucleic acid or polypeptide that has been removed from its natural cellular environment. An isolated nucleic acid is typically at least partially purified from other cellular nucleic acids, polypeptides and other constituents.

The term “functionally active” Retsat polypeptides refers to those fragments, derivatives and analogs displaying one or more known functional activities associated with a full-length (wild-type) Retsat polypeptide (e.g., converting all trans-retinol to all-trans (13,14)-dihydroretinol), antigenicity (binding to an anti-Retsat antibody), immunogenicity, and the like. Functionally active molecules include Retsat polypeptides, fragments, derivatives and analogs thereof, nucleic acids encoding Retsat polypeptides, fragments, and derivatives thereof, and anti-Retsat antibodies.

The term “therapeutically effective” refers to an amount of a molecule (e.g., a RetSat polypeptide, anti-RetSat antibody, RETSAT nucleic acid, all-trans-(13,14)-dihydroretinol, all-trans-13,14-dihydroretinoic acid and 13,14-dihydroretinoid derivatives that is sufficient to modulate cell proliferation, retinoid metabolism, skin and/or immune function and regulation in a subject, such as a patient or a mammal.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

The term “substantial similarity” in the context of polypeptide sequences, indicates that the polypeptide comprises a sequence with at least 70% sequence identity to a reference sequence, or preferably 80%, or more preferably 85% sequence identity to the reference sequence, or most preferably 90% identity over a comparison window of about 10-20 amino acid residues. In the context of amino acid sequences, “substantial simlarity” further includes conservative substitutions of amino acids. Thus, a polypeptide is substantially similar to a second polypeptide, for example, where the two peptides differ by one or more conservative substitutions.

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company, 1984.) In addition, individual substitutions, deletions or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman, Adv. Appl. Math. 2:482, 1981, which is incorporated by reference herein), by the homology alignment algorithm of Needleman and Wunsch, J. Mol. Biol. 48:443-53, 1970, which is incorporated by reference herein), by the search for similarity method of Pearson and Lipman, Proc. Natl. Acad. Sci. USA 85:2444-48, 1988, which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4thed., John Wiley and Sons, New York, 1999).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle, J. Mol. Evol. 25:351-60, 1987, which is incorporated by reference herein). The method used is similar to the method described by Higgins and Sharp, Comput. Appl. Biosci. 5:151-53, 1989, which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al., J. Mol. Biol. 215:403-410, 1990, which is incorporated by reference herein). (See also Zhang et al., Nucleic Acid Res. 26:3986-90, 1998; Altschul et al., Nucleic Acid Res. 25:3389-402, 1997, which are incorporated by reference herein). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. 1990, supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9, 1992, which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77, 1993, which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions.

The term “immunological cross-reactive” means that a polypeptide, fragment, derivative or analog is capable of competitively inhibiting the binding of an antibody to its antigen.

The terms “transformation” or “transfection” refer to the process of stably altering the genotype of a recipient cell or microorganism by the introduction of polynucleotides. This is typically detected by a change in the phenotype of the recipient cell or organism. The term “transformation” is generally applied to microorganisms, while “transfection” is used to describe this process in cells derived from multicellular organisms.

The term “sample” generally indicates a specimen of tissue, cells, plasma, serum, spinal fluid, lymph fluid, the external sections of the skin, respiratory, intestinal, and genitourinary tracts, tears, saliva, blood cells, hair, tumors, organs, other material of biological origin that contains polynucleotides, or in vitro cell culture constituents of any of these. A sample can further be semi-purified or purified forms of polynucleotides. A sample can be isolated from a mammal, such as a human, an animal, or any other organism having a RETSAT locus, as well as in vitro culture constituents of any of these.

The term “disease” refers to a disease, condition, or disorder associated with cell proliferation, retinoid metabolism, skin and/or immune function and regulation. Such diseases include, for example, cancer, blindness, skin diseases and conditions and immunological disorders.

Generally, other nomenclature used herein and many of the laboratory procedures in cell culture, molecular genetics and nucleic acid chemistry and hybridization, which are described below, are those well known and commonly employed in the art. (See generally Ausubel et al. 1999 supra; Sambrook et al., Molecular Cloning: A Laboratory Manual, 3d Ed., Cold Spring Harbor Laboratory Press, New York, 2001, which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

### The RETSAT Gene

The invention relates to the nucleotide sequences of encoding RetSat. The human, mouse and monkey (macaque) RETSAT DNAs were identified. In a specific embodiment, a RETSAT nucleic acid comprises a nucleic acid of human RetSat (GenBank Accession Number gi46329587), mouse RetSat (GenBank Accession Number AY704159) and monkey (macaque) RetSat (GenBank Accession Number AY707524), or the coding region of the RETSAT locus, or nucleic acid sequences (e.g., an open reading frame) encoding a Retsat polypeptide (e.g., human RetSat (GenBank Accession Number gi46329587), mouse RetSat (GenBank Accession Number AY704159) and monkey (macaque) RetSat (GenBank Accession Number AY707524).). RETSAT nucleic acids further include mRNAs, genomic DNA, and antisense nucleic acids corresponding to the RETSAT locus. RETSAT nucleic acids further include derivatives (e.g., nucleotide sequence variants), such as those encoding other possible codon choices for the same amino acid or conservative amino acid substitutions thereof, such as naturally occurring allelic variants. Due to the degeneracy of nucleotide coding sequences, other DNA sequences which encode substantially the same amino acid sequence as a RETSAT gene (human RetSat (GenBank Accession Number gi46329587), mouse RetSat (GenBank Accession Number AY704159) and monkey (macaque) RetSat (GenBank Accession Number AY707524)), can be used in the practice of the present invention. These include, but are not limited to, nucleotide sequences comprising all or portions of a RETSAT gene which is altered by the substitution of different codons that encode the same or a functionally equivalent amino acid residue (e.g., a conservative substitution) within the sequence, thus producing a silent change.

The invention also provides RETSAT nucleic acid fragments of at least 6 contiguous nucleotides (e.g., a hybridizable portion); in other embodiments, the nucleic acids comprise at least 8 contiguous nucleotides, 25 nucleotides, 50 nucleotides, 100 nucleotides, 150 nucleotides, 200 nucleotides, or even up to 250 nucleotides or more of a RETSAT sequence. In another embodiment, the nucleic acids are smaller than 200 or 250 nucleotides in length. The RETSAT nucleic acids can be single or double-stranded. As is readily apparent, as used herein, a “nucleic acid encoding a fragment of an Retsat polypeptide” is construed as referring to a nucleic acid encoding only the recited fragment or portion of the Retsat polypeptide and not the other contiguous portions of the Retsat polypeptide as a contiguous sequence. Fragments of RETSAT nucleic acids encoding one or more Retsat domains are also provided.

RETSAT nucleic acids further include those nucleic acids hybridizable to, or complementary to, the foregoing sequences. In specific aspects, nucleic acids are provided which comprise a sequence complementary to at least 10, 25, 50, 100, 200, or 250 nucleotides or more of a RETSAT gene. In a specific embodiment, a nucleic acid which is hybridizable to a RETSAT nucleic acid (e.g., having sequence SEQ ID NO:1), or to a nucleic acid encoding a RETSAT derivative, under conditions of low, medium or high stringency, is provided.

By way of example, and not limitation, procedures using low stringency conditions are as follows: Filters containing DNA are pretreated for 6 hours at 40° C. in a solution containing 35% formamide, 5×SSC, 50 mM Tris-HCl (pH 7.5), 5 mM EDTA, 0.1% polyvinylpyrrolidone (PVP), 0.1% Ficoll, 1% bovine serum albumin (BSA), and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 cpm 32P-labeled probe. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and re-exposed to film. Other conditions of low stringency that can be used are well known in the art (e.g., those employed for cross-species hybridizations). (See also Shilo et al. Weinberg, Proc. Natl. Acad. Sci. USA 78:6789-92, 1981).

In another embodiment, a nucleic acid which is hybridizable to a RETSAT nucleic acid under conditions of high stringency is provided. By way of example, and not limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 □g/ml denatured salmon sperm DNA. Filters are hybridized for 48 hours at 65° C. in prehybridization mixture containing 100 μg/ml denatured salmon sperm DNA and 5-20×106 cpm of 32P-labeled probe. Washing of filters is done at 65° C. for 1 hour in a solution containing 2×SSC, 0.01% PVP, 0.01% Ficoll, and 0.01% BSA. This is followed by a wash in 0.1×SSC at 50° C. for 45 min before autoradiography. Other conditions of high stringency which can be used are well known in the art. (See generally Ausubel et al., supra; Sambrook et al., supra).

In another specific embodiment, a nucleic acid which is hybridizable to a RETSAT nucleic acid under conditions of moderate stringency is provided. By way of example, and not limitation, procedures using such conditions of moderate stringency are as follows: Prehybridization of filters containing DNA is carried out for 8 hours to overnight at 55° C. in buffer composed of 6×SSC, 50 mM Tris-HCl (pH 7.5), 1 mM EDTA, 0.02% PVP, 0.2% Ficoll, 0.02% BSA and 500 μg/ml denatured salmon sperm DNA. Filters are hybridized for 24 hours at 55° C. in a prehybridization mixture containing 100 μg/ml denatured salmon sperm DNA and 5-20×106 cpm of 32P-labeled probe. Washing of filters is done at 37° C. for 1 hour in a solution containing 2×SSC, 0.01% PVP, 0.01% Ficoll, and 0.01% BSA.

Various other stringency conditions which promote hybridization can be used. For example, hybridization in 6×SSC at about 45° C., followed by washing in 2×SSC at 50° C. can be used. Alternatively, the salt concentration in the wash step can range from low stringency of about 5×SSC at 50° C., to moderate stringency of about 2×SSC at 50° C., to high stringency of about 0.2×SSC at 50° C. In addition, the temperature of the wash step can be increased from low stringency conditions at room temperature, to moderately stringent conditions at about 42° C., to high stringency conditions at about 65° C. Other conditions include, but are not limited to, hybridizing at 68° C. in 0.5M NaH₂PO₄ (pH 7.2)/1 mM EDTA/7% SDS, or hybridization in 50% formamide/0.25M NaH₂PO₄ (pH 7.2)/0.25 M NaCl/1 mM EDTA/7% SDS, followed by washing in 40 mM NaH₂PO₄ (pH 7.2)/1 mM EDTA/5% SDS at 50° C. or in 40 mM NaH₂PO₄ (pH 7.2)/1 mM EDTA/1% SDS at 50° C. Both temperature and salt can be varied, or alternatively, one or the other variable may remain constant while the other is changed.

Low, moderate and high stringency conditions are well known to those of skill in the art, and will vary predictably depending on the base composition of the particular nucleic acid sequence and on the specific organism from which the nucleic acid sequence is derived. For guidance regarding such conditions see, for example, Sambrook et al. (supra); and Ausubel et al. (supra).

RETSAT nucleic acids further include derivatives and analogs. Such derivatives and analogs can comprise at least one modified base moiety, such as, for example, 5-fluorouracil, 5-bromouracil, 5-chlorouracil, 5-iodouracil, hypoxanthine, xanthine, 4-acetylcytosine, 5-(carboxy-hydroxylmethyl)uracil, 5-carboxymethylaminomethyl-2-thiouridine, 5-carboxymethylamino-methyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, 2,6-diaminopurine, and the like. The RETSAT nucleic acids can also have at least one modified sugar moiety, such as, for example, arabinose, 2-fluoroarabinose, xylulose, and hexose.

The RETSAT nucleic acids can also have a modified phosphate backbone, such as, for example, a phosphorothioate, a phosphorodithioate, a phosphoramidothioate, a phosphoramidate, a phosphordiamidate, a methylphosphonate, an alkyl phosphotriester, and a formacetal or analog thereof.

The RETSAT nucleic acids can also be an a-anomeric oligonucleotide. An α-anomeric oligonucleotide forms specific double-stranded hybrids with complementary RNA in which, contrary to the usual P-units, the strands run parallel to each other (see, e.g., Gautier et al., Nucl. Acids Res. 15:6625-41, 1987).

RETSAT nucleic acid derivatives or analogs can be synthesized by standard methods known in the art (e.g., by use of a commercially available automated DNA synthesizer). As examples, phosphorothioate nucleic acids can be synthesized by the method of Stein et al. Nucl. Acids Res. 16:3209-21, 1988, and methyphosphonate nucleic acids oligonucleotides can be prepared by use of controlled pore glass polymer supports (Sarin et al., Proc. Natl. Acad. Sci. USA 85:7448-51, 1988), and the like.

Specific embodiments for the isolation of RETSAT nucleic acids, presented as example but not by way of limitation, are as follows.

For expression cloning (a technique commonly known in the art), an expression library is constructed by methods known in the art. For example, mRNA (e.g., human) is isolated, cDNA is prepared and then ligated into an expression vector (e.g., a bacteriophage derivative) such that it is capable of being expressed by the host cell into which it is then introduced. Various screening assays can then be used to select for the expressed Retsat polypeptide. In one embodiment, anti-Retsat specific antibodies can be used for selection.

In another embodiment, polymerase chain reaction (PCR) can be used to amplify the desired sequence in a genomic or cDNA library, prior to selection. Oligonucleotide primers representing known RETSAT sequences, for example, as selected from human RetSat (GenBank Accession Number gi46329587), mouse RetSat (GenBank Accession Number AY704159) and monkey (macaque) RetSat (GenBank Accession Number AY707524), can be used as primers in PCR. In a typical embodiment, the oligonucleotide primers represent at least part of the RETSAT conserved segments of strong identity between RETSAT of different species. The synthetic oligonucleotides can be utilized as primers to amplify particular oligonucleotides within the RETSAT gene by PCR sequences from a source (RNA or DNA), typically a cDNA library, of potential interest. PCR can be carried out, for example, by use of a Perkin-Elmer Cetus thermal cycler and Taq polymerase (Gene Amp). The DNA being amplified can include mRNA or cDNA or genomic DNA from any eukaryotic species. One of skill in the art can choose to synthesize several different degenerate primers for use in the PCR reactions.

It is also possible to vary the stringency of hybridization conditions used in priming the PCR reactions, to allow for greater or lesser degrees of nucleotide sequence similarity between the known RETSAT nucleotide sequence and the related nucleic acid being isolated. For cross species hybridization, low stringency conditions are typically used. For same species hybridization, moderately stringent conditions are more typically used. After successful amplification of a segment of a related RETSAT nucleic acid, that segment can be molecularly cloned and sequenced, and utilized as a probe to isolate a complete cDNA or genomic clone. This, in turn, can permit the determination of the gene's complete nucleotide sequence, the analysis of its expression, and the production of its polypeptide product for functional analysis, as described infra. In this fashion, additional genes encoding Retsat polypeptides and Retsat polypeptide derivatives can be identified.

The above-methods are not meant to limit the following general description of methods by which clones of RETSAT nucleic acids or fragments can be obtained. Any eukaryotic cell potentially can serve as the nucleic acid source for the molecular cloning of the RETSAT gene. The nucleic acid sequences encoding RETSAT can be isolated from vertebrate sources including, mammalian sources such as, porcine, bovine, feline, avian, equine, canine and human, as well as additional primate, avian, reptilian, amphibian, and piscine sources, and the like, from non-vertebrate sources, such as insects, worms, nematodes, plants, and the like. The DNA can be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., supra; Glover, (ed.), DNA Cloning: A Practical Approach, IRL Press, Washington, D.C. Vol. I, II, 1985.) Clones derived from genomic DNA can contain regulatory and intron DNA regions in addition to coding regions; clones derived from cDNA will typically contain only exon sequences. Whatever the source, the nucleic acids can be molecularly cloned into a suitable vector for propagation of those nucleic acids.

In the molecular cloning of the gene from genomic DNA, DNA fragments are generated, some of which will encode a RETSAT gene. The DNA can be cleaved at specific sites using various restriction enzymes. Alternatively, one can use DNase in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific nucleic acid containing the desired gene can be accomplished in a number of ways. For example, a portion of a RETSAT (of any species) gene or its specific RNA, or a fragment thereof can be purified and labeled. The generated DNA fragments can be screened by nucleic acid hybridization to the labeled probe (see, e.g., Benton and Davis, Science 196:180-02, 1977; Grunstein and Hogness, Proc. Natl. Acad. Sci. USA 72:3961-65, 1975). Those DNA fragments with substantial identity to the probe will hybridize. It is also possible to identify the appropriate fragment by restriction enzyme digestion(s) and comparison of fragment sizes with those expected according to a known restriction map, if such is available. Further selection can be carried out on the basis of the properties of the gene.

Alternatively, the presence of the RETSAT nucleic acids can be detected by assays based on the physical, chemical, or immunological properties of its expressed product. For example, cDNA clones, or DNA clones which hybrid-select the proper mRNAs, can be selected which produce a polypeptide that, for example, has similar or identical electrophoretic migration, isoelectric focusing behavior, proteolytic digestion maps, RetSat activity, substrate binding activity, or antigenic properties as known for Retsat polypeptide(s). Immune serum or antibody which specifically binds to the Retsat polypeptide can be used to identify putatively Retsat polypeptide synthesizing clones by binding in an immunoassay, (e.g. an ELISA (enzyme-linked immunosorbent assay)-type procedure).

The RETSAT gene can also be identified by mRNA selection by nucleic acid hybridization followed by in vitro translation. In this procedure, fragments are used to isolate complementary mRNAs by hybridization. Such DNA fragments typically represent available, purified RETSAT DNA of another species (e.g., human, mouse, and the like). Immunoprecipitation analyses or functional assays of the in vitro translation products of the isolated mRNAs identifies the mRNA and, therefore, the complementary DNA fragments that contain the desired sequences. In addition, specific mRNAs can be selected by adsorption of polysomes isolated from cells to immobilized antibodies specifically directed against Retsat polypeptide. A radiolabeled RETSAT cDNA can be synthesized using the selected mRNA (from the adsorbed polysomes) as a template. The radiolabeled mRNA or cDNA can then be used as a probe to identify the RETSAT DNA from among other genomic DNA.

Alternatives to isolating the RETSAT genomic DNA include, but are not limited to, chemically synthesizing the gene sequence itself from a known sequence or making cDNA to the mRNA which encodes the Retsat polypeptide. For example, RNA for cDNA cloning of the RETSAT gene can be isolated from cells that express the Retsat polypeptide. Other methods are possible and are considered within the scope of the invention.

The identified and isolated RETSAT nucleic acids can then be inserted into an appropriate cloning vector. A large number of vector-host systems known in the art can be used. Possible vectors include, but are not limited to, plasmids or modified viruses. The vector system is selected to be compatible with the host cell. Such vectors include, but are not limited to, bacteriophages such as lambda derivatives, yeast integrative and centromeric vectors, 2μ plasmid, and derivatives thereof, or plasmids such as pBR322, pUC, pcDNA3.1 or pRSET (Invitrogen) plasmid derivatives or the Bluescript vector (Stratagene), to name but a few. The insertion of the RETSAT nucleic acids into a cloning vector can, for example, be accomplished by ligating the DNA fragment into a cloning vector which has complementary cohesive termini. If the complementary restriction sites used to fragment the DNA are not present in the cloning vector, however, the ends of the DNA molecules can be enzymatically modified. Alternatively, any desired restriction endonuclease site can be produced by ligating nucleotide sequences (e.g., linkers) onto the DNA termini; these ligated sequences can comprise specific chemically synthesized oligonucleotides encoding restriction endonuclease recognition sequences. In an alternative method, the cleaved vector and RETSAT nucleic acids can be modified by homopolymeric tailing. Recombinant molecules can be introduced into host cells via transformation, transfection, infection, electroporation, and the like, so that many copies of the nucleic acid sequence are generated.

In an alternative method, the RETSAT nucleic acids can be identified and isolated after insertion into a suitable cloning vector in a “shot gun” approach. Enrichment for the RETSAT nucleic acids, for example, by size fractionation, can be done before insertion into the cloning vector. In specific embodiments, transformation of host cells with recombinant DNA molecules that incorporate the isolated RETSAT gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

### Expression of the RETSAT Gene

The nucleotide sequence coding for a Retsat polypeptide, or a functionally active derivative, analog or fragment thereof, can be inserted into an appropriate expression vector (i.e., a vector which contains the necessary elements for the transcription and translation of the inserted polypeptide-coding sequence). The necessary transcriptional and translational signals can also be supplied by the native RETSAT gene and/or its flanking regions. A variety of host-vector systems can be utilized to express the polypeptide-coding sequence. These include, but are not limited to, mammalian cell systems infected with virus (e.g., vaccinia virus, adenovirus, and the like), insect cell systems infected with virus (e.g., baculovirus), microorganisms such as yeast containing yeast vectors, or bacteria transformed with bacteriophage DNA, plasmid DNA, or cosmid DNA. The expression elements of vectors vary in their strengths and specificities. Depending on the host-vector system utilized, any one of a number of suitable transcription and translation elements can be used. In specific embodiments, the human RETSAT gene is expressed, or a nucleic acid sequence encoding a functionally active portion of human Retsat is expressed in yeast or bacteria. In yet another embodiment, a fragment of RETSAT comprising a domain of the Retsat polypeptide is expressed.

Any of the methods previously described for the insertion of DNA fragments into a vector can be used to construct expression vectors containing a chimeric gene consisting of appropriate transcriptional/translational control signals and the polypeptide coding sequences. These methods include in vitro recombinant DNA and synthetic techniques and in vivo recombinants (genetic recombination). Expression of nucleic acid sequences encoding a Retsat polypeptide or fragment can be regulated by a second nucleic acid sequence so that the Retsat polypeptide or fragment is expressed in a host transformed with the recombinant DNA molecule. For example, expression of a Retsat polypeptide can be controlled by any promoter/enhancer element known in the art. Promoters which can be used to control RETSAT gene expression include, but are not limited to, the SV40 early promoter region (Benoist and Chambon, Nature 290:304-10, 1981), the promoter contained in the 3′ long terminal repeat of Rous sarcoma virus (Yamamoto et al., Cell 22:787-97, 1980), the herpes thymidine kinase promoter (Wagner et al., Proc. Natl. Acad. Sci. USA 78:1441-45, 1981), the regulatory sequences of the metallothionein gene (Brinster et al., Nature 296:39-42, 1982), prokaryotic expression vectors such as the beta-lactamase promoter (Villa-Komaroff et al., Proc. Natl. Acad. Sci. USA 75:3727-31, 1978) or the tac promoter (deBoer et al., Proc. Nat. Acad. Sci. USA 80:21-25, 1983), plant expression vectors including the cauliflower mosaic virus 35S RNA promoter (Gardner et al., Nucl. Acids Res. 9:2871-88, 1981), and the promoter of the photosynthetic enzyme ribulose biphosphate carboxylase (Herrera-Estrella et al., Nature 310:115-20, 1984), promoter elements from yeast or other fungi such as the Gal7 and Gal4 promoters, the ADH (alcohol dehydrogenase) promoter, the PGK (phosphoglycerol kinase) promoter, the alkaline phosphatase promoter, and the like.

The following animal transcriptional control regions, which exhibit tissue specificity, have been utilized for transgenic expression animals: the elastase I gene control region which is active in pancreatic acinar cells (Swift et al., Cell 38:639-46, 1984; Omitz et al., Cold Spring Harbor Symp. Quant. Biol. 50:399-409, 1986; MacDonald, Hepatology 7(1 Suppl.):42S-51S, 1987; the insulin gene control region which is active in pancreatic beta cells (Hanahan, Nature 315:115-22, 1985), the immunoglobulin gene control region which is active in lymphoid cells (Grosschedl et al., Cell 38:647-58, 1984; Adams et al., Nature 318:533-8, 1985; Alexander et al., Mol. Cell. Biol. 7:1436-44, 1987), the mouse mammary tumor virus control region which is active in testicular, breast, lymphoid and mast cells (Leder et al., Cell 45:485-95, 1986), the albumin gene control region which is active in liver (Pinkert et al., Genes Dev. 1:268-76, 1987), the alpha-fetoprotein gene control region which is active in liver (Krumlauf et al., Mol. Cell. Biol. 5:1639-48, 1985; Hammer et al., Science 235:53-58, 1987); the alpha 1-antitrypsin gene control region which is active in the liver (Kelsey et al., Genes and Devel. 1:161-71, 1987); the beta-globin gene control region which is active in myeloid cells (Magram et al., Nature 315:338-40, 1985; Kollias et al., Cell 46:89-94, 1986); the myelin basic protein gene control region which is active in oligodendrocyte cells in the brain (Readhead et al., Cell 48:703-12, 1987); the myosin light chain-2 gene control region which is active in skeletal muscle (Shani, Nature 314:283-86, 1985); and the gonadotropic releasing hormone gene control region which is active in the hypothalamus (Mason et al., Science 234:1372-78, 1986).

In a specific embodiment, a vector is used that comprises a promoter operably linked to a RetSat-encoding nucleic acid, one or more origins of replication, and, optionally, one or more selectable markers (e.g., an antibiotic resistance gene). For example, an expression construct can be made by subcloning a RETSAT coding sequence into a restriction site of the pRSECT expression vector. Such a construct allows for the expression of the Retsat polypeptide under the control of the T7 promoter with a histidine amino terminal flag sequence for affinity purification of the expressed polypeptide.

Expression vectors containing RETSAT nucleic acid inserts can be identified by general approaches well known to the skilled artisan, including: (a) nucleic acid hybridization, (b) the presence or absence of “marker” gene function, and (c) expression of inserted sequences. In the first approach, the presence of a RETSAT nucleic acid inserted in an expression vector can be detected by nucleic acid hybridization using probes comprising sequences that are homologous to an inserted RETSAT nucleic acid. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, and the like) caused by the insertion of a vector containing the RETSAT nucleic acids. For example, if the RETSAT nucleic acid is inserted within the marker gene sequence of the vector, recombinants containing the RETSAT insert can be identified by the absence of marker gene function.

In the third approach, recombinant expression vectors can be identified by assaying the Retsat polypeptide expressed by the recombinant. Such assays can be based, for example, on the physical or functional properties of the Retsat polypeptide in in vitro assay systems. Once a particular recombinant DNA molecule is identified and isolated, several methods that are known in the art can be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain can be chosen that modulates the expression of the inserted sequences, or modifies or processes the gene product in the specific fashion desired. Expression from certain promoters can be elevated in the presence of certain inducers; thus, expression of the genetically engineered Retsat polypeptide can be controlled. Furthermore, different host cells having characteristic and specific mechanisms for the translational and post-translational processing and modification (e.g., glycosylation, phosphorylation) of polypeptides can be used. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a mammalian protein. Furthermore, different vector/host expression systems can affect processing reactions to different extents.

### Retsat Polypeptides, Fragments, Derivatives and Analogs

The invention further relates to Retsat polypeptides, fragments, derivatives and analogs thereof. In one aspect, the invention provides amino acid sequences of Retsat polypeptide, typically Retsat polypeptide (encoded by human RetSat (GenBank Accession Number gi46329587), mouse RetSat (GenBank Accession Number AY704159) and monkey (macaque) RetSat (GenBank Accession Number AY707524)). In particular aspects, the polypeptides, fragments, derivatives, or analogs of Retsat polypeptides are from an animal (e.g., human, mouse, rat, pig, cow, dog, monkey, and the like). The production and use of Retsat polypeptides, fragments, derivatives and analogs thereof are also within the scope of the present invention. In a specific embodiment, the fragment, derivative or analog is functionally active (i.e., capable of exhibiting one or more functional activities associated with a full-length, wild-type Retsat polypeptide). As one example, such fragments, derivatives or analogs which have the desired immunogenicity or antigenicity can be used, for example, in immunoassays, for immunization, for inhibition of Retsat activity, and the like. Fragments, derivatives or analogs that retain, or alternatively lack or inhibit, a desired Retsat property of interest (e.g., conversion of all-trans-retinol to all-trans-(13,14)-dihydroretinol) can be used as inducers, or inhibitors of such property and its physiological correlates. A specific embodiment relates to a Retsat fragment that can be bound by an anti-Retsat antibody. Fragments, derivatives or analogs of Retsat can be tested for the desired activity by procedures known in the art, including but not limited to the functional assays described herein.

Retsat polypeptide derivatives include naturally-occurring amino acid sequence variants as well as those altered by substitution, addition or deletion of one or more amino acid residues that provide for functionally active molecules. Retsat polypeptide derivatives include, but are not limited to, those containing as a primary amino acid sequence of all or part of the amino acid sequence of a Retsat polypeptide including altered sequences in which one or more functionally equivalent amino acid residues (e.g., a conservative substitution) are substituted for residues within the sequence, resulting in a silent change.

In another aspect, a polypeptide consisting of or comprising a fragment of a Retsat polypeptide having at least 10 contiguous amino acids of the Retsat polypeptide is provided. In other embodiments, the fragment consists of at least 20 or 50 contiguous amino acids of the Retsat polypeptide. In a specific embodiment, the fragments are not larger than 35, 100 or even 200 amino acids.

Fragments, derivatives or analogs of Retsat polypeptide include but are not limited to those molecules comprising regions that are substantially similar to Retsat polypeptide or fragments thereof (e.g., in various embodiments, at least 50%, 60%, 70%, 75%, 80%, 90%, or even 95% identity or similarity over an amino acid sequence of identical size), or when compared to an aligned sequence in which the alignment is done by a computer sequence comparison/alignment program known in the art, or whose coding nucleic acid is capable of hybridizing to a RETSAT nucleic acid, under high stringency, moderate stringency, or low stringency conditions (supra). Retsat polypeptides further comprise fragments and derivatives having an antigenic determinant (e.g., can be recognized by an antibody specific for human Retsat polypeptide).

The Retsat polypeptide derivatives and analogs can be produced by various methods known in the art. The manipulations which result in their production can occur at the gene or protein level. For example, the cloned RETSAT nucleic acids can be modified by any of numerous strategies known in the art (see, e.g., Sambrook et al., supra), such as making conservative substitutions, deletions, insertions, and the like. The sequence can be cleaved at appropriate sites with restriction endonuclease(s), followed by further enzymatic modification if desired, isolated, and ligated in vitro. In the production of the RETSAT nucleic acids encoding a fragment, derivative or analog of a Retsat polypeptide, the modified nucleic acid typically remains in the proper translational reading frame, so that the reading frame is not interrupted by translational stop signals or other signals which interfere with the synthesis of the Retsat fragment, derivative or analog. The RETSAT nucleic acid can also be mutated in vitro or in vivo to create and/or destroy translation, initiation and/or termination sequences. The Retsat encoding nucleic acid can also be mutated to create variations in coding regions and/or to form new restriction endonuclease sites or destroy preexisting ones and to facilitate further in vitro modification. Any technique for mutagenesis known in the art can be used, including but not limited to, chemical mutagenesis, in vitro site-directed mutagenesis (Hutchison et al., J. Biol. Chem. 253:6551-60, 1978), the use of TAB linkers (Pharmacia), and the like.

Manipulations of the Retsat polypeptide sequence can also be made at the polypeptide level. Included within the scope of the invention are Retsat polypeptide fragments, derivatives or analogs which are differentially modified during or after synthesis (e.g., in vivo or in vitro translation). Such modifications include conservative substitution, glycosylation, acetylation, phosphorylation, amidation, derivatization by known protecting/blocking groups, proteolytic cleavage, linkage to an antibody molecule or other cellular ligand, and the like. Any of numerous chemical modifications can be carried out by known techniques, including, but not limited to, specific chemical cleavage (e.g., by cyanogen bromide), enzymatic cleavage (e.g., by trypsin, chymotrypsin, papain, V8 protease, and the like); modification by, for example, NaBH4 acetylation, formylation, oxidation and reduction, or metabolic synthesis in the presence of tunicamycin, and the like.

In addition, fragments, derivatives and analogs of Retsat polypeptides can be chemically synthesized. For example, a peptide corresponding to a portion, or fragment, of a Retsat polypeptide, which comprises a desired domain, or which mediates a desired activity in vitro, can be synthesized by use of chemical synthetic methods using, for example, an automated peptide synthesizer. Furthermore, if desired, nonclassical amino acids or chemical amino acid analogs can be introduced as a substitution or addition into the Retsat polypeptide sequence. Non-classical amino acids include but are not limited to the D-isomers of the common amino acids, alpha-amino isobutyric acid, 4-aminobutyric acid, 2-amino butyric acid, .gamma.-amino butyric acid, epsilon-Ahx, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, beta-alanine, selenocysteine, fluoro-amino acids, designer amino acids such as beta-methyl amino acids, C alpha-methyl amino acids, N alpha-methyl amino acids, and amino acid analogs in general. Furthermore, the amino acid can be D (dextrorotary) or L (levorotary).

In a specific embodiment, the Retsat fragment or derivative is a chimeric, or fusion, protein comprising a Retsat polypeptide or fragment thereof (typically consisting of at least a domain or motif of the Retsat polypeptide, or at least 10 contiguous amino acids of the Retsat polypeptide) joined at its amino- or carboxy-terminus via a peptide bond to an amino acid sequence of a different protein. In one embodiment, such a chimeric protein is produced by recombinant expression of a nucleic acid encoding the protein. The chimeric product can be made by ligating the appropriate nucleic acid sequence, encoding the desired amino acid sequences, to each other in the proper coding frame and expressing the chimeric product by methods commonly known in the art. Alternatively, the chimeric product can be made by protein synthetic techniques (e.g., by use of an automated peptide synthesizer).

Retsat polypeptides can be isolated and purified by standard methods including chromatography (e.g., ion exchange, affinity, sizing column chromatography, high pressure liquid chromatography), centrifugation, differential solubility, or by any other standard technique for the purification of proteins. The functional properties can be evaluated using any suitable assay as described herein or otherwise known to the skilled artisan. Alternatively, once a Retsat polypeptide produced by a recombinant is identified, the amino acid sequence of the polypeptide can be deduced from the nucleotide sequence of the chimeric gene contained in the recombinant. As a result, the protein can be synthesized by standard chemical methods known in the art (see, e.g., Hunkapiller et al., Nature 310:105-11, 1984; Stewart and Young, Solid Phase Peptide Synthesis, 2nd Ed., Pierce Chemical Co., Rockford, Ill., 1984).

In another alternate embodiment, native Retsat polypeptides can be purified from natural sources by standard methods such as those described above (e.g., immunoaffinity purification). In a specific embodiment of the present invention, Retsat polypeptides, whether produced by recombinant DNA techniques, by chemical synthetic methods or by purification of native polypeptides, include but are not limited to those containing as a primary amino acid sequence all or part of the amino acid sequence of human Retsat polypeptide (SEQ ID NO:2), as well as fragments, derivatives and analogs thereof.

### Structure of the RETSAT Gene and Polypeptide(s)

The structure of the RETSAT gene and Retsat polypeptide can be analyzed by various methods known in the art. The cloned DNA or cDNA corresponding to the RETSAT gene can be analyzed by methods including but not limited to Southern hybridization (Southern, J. Mol. Biol. 98:503-17, 1975), Northern hybridization (see, e.g., Freeman et al., Proc. Natl. Acad. Sci. USA 80:4094-98, 1983), restriction endonuclease mapping (see generally Sambrook et al., supra), and DNA sequence analysis (see, e.g., Sambrook et al., supra). Polymerase chain reaction (PCR; see, e.g., U.S. Pat. Nos. 4,683,202, 4,683,195 and 4,889,818; Gyllensten et al., Proc. Natl. Acad. Sci. USA 85:7652-56, 1988; Ochman et al., Genetics 120:621-3, 1988; Loh et al., Science 243:217-20, 1989) followed by Southern hybridization with a RETSAT-specific probe can allow the detection of the RETSAT gene in DNA from various cell types. Methods of amplification other than PCR are commonly known and can also be employed.

In one embodiment, Southern blot hybridization can be used to determine the genetic linkage of the RETSAT locus. Northern blot hybridization analysis can be used to determine the expression of the RETSAT gene. Various cell types at various states of development or activity can be tested for RETSAT expression. The stringency of the hybridization conditions for both Southern and Northern blot hybridization can be manipulated to ensure detection of nucleic acids with the desired degree of sequence identity to the specific RETSAT probe used. Modifications of these and other methods commonly known in the art can be used. Restriction endonuclease mapping can be used to roughly determine the genetic structure of the RETSAT gene. Restriction maps derived by restriction endonuclease cleavage can be confirmed by DNA sequence analysis. DNA sequence analysis can be performed by any techniques known in the art, including but not limited to the method of Maxam and Gilbert, Meth. Enzymol. 65:499-560, 1980), the Sanger dideoxy method (Sanger et al., Proc. Natl. Acad. Sci. USA 74:5463-67, 1977), the use of T7 DNA polymerase (Tabor and Richardson, U.S. Pat. No. 4,795,699), or use of an automated DNA sequencer (e.g., Applied Biosystems, Foster City, Calif.).

The amino acid sequence of the Retsat polypeptide can be derived by deduction from the DNA sequence, or alternatively, by direct sequencing of the protein (e.g., with an automated amino acid sequencer). The Retsat polypeptide sequence can be further characterized by a hydrophilicity analysis (Hopp and Woods, Proc. Natl. Acad. Sci. USA 78:3824-28, 1981). A hydrophilicity profile can be used to identify the hydrophobic and hydrophilic regions of the Retsat polypeptide and the corresponding regions of the gene sequence which encode such regions.

Secondary structural analysis (e.g., Chou and Fasman, Biochemistry 13:222-45, 1974) can also be conducted to identify regions of the Retsat polypeptide that assume specific secondary structures. Manipulation, translation, and secondary structure prediction, open reading frame prediction and plotting, as well as determination of sequence identity and similarities, can also be accomplished using computer software programs available in the art, such as those described above. Other methods of structural analysis can also be employed. These include but are not limited to X-ray crystallography (Engstom, Biochem. Exp. Biol. 11:7-13, 1974) and computer modeling (Fletterick and Zoller, (eds.), “Computer Graphics and Molecular Modeling”, In Current Communications in Molecular Biology, Cold Spring Harbor Laboratory, Cold Spring Harbor, N.Y., 1986; Bordo, Comput. Appl. Biosci. 9:639-45, 1993; Bruccoleri and Karpus, Biopolymers 26:137-68, 1987; Hansen et al. Pac. Symp. Biocoput. 106-17, 1998); Li et al., Protein Sci. 6:956-70, 1997; Stemberg and Zvelebil, Eur. J. Cancer 26:1163-66, 1990; Ring and Cohen, FASEB J. 7:783-90, 1993; and Sutcliffe et al., Protein Eng. 1:377-84, 1987).

Antibodies to Retsat Polypeptides, Fragments. Derivatives and Analogs

Retsat polypeptides, fragments, derivatives, and analogs thereof, can be used as an immunogen to generate antibodies which immunospecifically bind such Retsat polypeptides, fragments, derivatives, and analogs thereof. Such antibodies include but are not limited to polyclonal antibodies, monoclonal antibodies, chimeric antibodies, single chain antibodies, heavy chain antibody fragments (e.g., F(ab′), F(ab′)2, Fv, or hypervariable regions), and an Fab expression library. In a specific embodiment, polyclonal and/or monoclonal antibodies to whole, intact human Retsat polypeptide are produced. In another embodiment, antibodies to a domain of a human Retsat polypeptide are produced. In another embodiment, fragments of a human Retsat polypeptide identified as hydrophilic are used as immunogens for antibody production.

Methods for making and using antibodies are generally disclosed by Harlow and Lane (Using Antibodies, A Laboratory Manual, Cold Spring Harbor Laboratory, Cold Spring Harbor, N.Y., 1999; the disclosure of which is incorporated by reference herein). Various procedures known in the art can be used for the production of polyclonal antibodies to a Retsat polypeptide, fragment, derivative or analog thereof. For the production of such antibodies, various host animals (including, but not limited to, rabbits, mice, rats, sheep, goats, camals, llamas and the like) can be immunized by injection with the native Retsat polypeptide, fragment, derivative or analog. Various adjuvants can be used to increase the immunological response, depending on the host species, including but not limited to Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward a Retsat polypeptide, fragment, derivative, or analog thereof, any technique which provides for the production of antibody molecules by continuous cell lines in culture can also be used. Such techniques include, for example, the hybridoma technique originally developed by Kohler and Milstein (see, e.g., Nature 256:495-97, 1975), as well as the trioma technique, (see, e.g., Hagiwara and Yuasa, Hum. Antibodies Hybridomas 4:15-19, 1993), the human B-cell hybridoma technique (see, e.g., Kozbor et al., Immunology Today 4:72, 1983), and the EBV-hybridoma technique to produce human monoclonal antibodies (see, e.g., Cole et al., In Monoclonal Antibodies and Cancer Therapy, Alan R. Liss, Inc., pp. 77-96, 1985). Human antibodies can be used and can be obtained by using human hybridomas (see, e.g., Cote et al., Proc. Natl. Acad. Sci. USA 80:2026-30, 1983) or by transforming human B cells with EBV virus in vitro (see, e.g., Cole et al., supra).

Further to the invention, “chimeric” or “humanized” antibodies (see, e.g., Morrison et al., Proc. Natl. Acad. Sci. USA 81:6851-5, 1984; Neuberger et al., Nature 312:604-08, 1984; Takeda et al., Nature 314:452-4, 1985) can be prepared. Such chimeric antibodies are typically prepared by splicing the non-human genes for an antibody molecule specific for a Retsat polypeptide together with genes from a human antibody molecule of appropriate biological activity. It can be desirable to transfer the antigen binding regions (e.g., F(ab′)2, F(ab′), Fv, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567; 4,816,397; 5,693,762; and 5,712,120; International Patent Publications WO 87/02671 and WO 90/00616; and European Patent Publication EP 239 400; the disclosures of which are incorporated by reference herein). Alternatively, a human monoclonal antibody or portions thereof can be identified by first screening a human B-cell cDNA library for DNA molecules that encode antibodies that specifically bind to an Retsat polypeptide according to the method generally set forth by Huse et al., Science 246:1275-81, 1989. The DNA molecule can then be cloned and amplified to obtain sequences that encode the antibody (or binding domain) of the desired specificity. Phage display technology offers another technique for selecting antibodies that bind to Retsat polypeptides, fragments, derivatives or analogs thereof. (See, e.g., International Patent Publications WO 91/17271 and WO 92/01047; and Huse et al., supra).

According to another aspect of the invention, techniques described for the production of single chain antibodies (see, e.g., U.S. Pat. Nos. 4,946,778 and 5,969,108) can be adapted to produce RetSat-specific single chain antibodies. An additional aspect of the invention utilizes the techniques described for the construction of a Fab expression library (see, e.g., Huse et al. 1989 supra) to allow rapid and easy identification of monoclonal Fab fragments with the desired specificity for Retsat polypeptides, fragments, derivatives, or analogs thereof.

The immunoglobulins also can be heavy chain antibodies. Immunoglobulins from animals such as camels, dromedaries, and llamas (Tylopoda) can form heavy chain antibodies, which comprise heavy chains without light chains. (See, e.g., Desmyter et al., J. Biol. Chem. 276:26285-90, 2001; Muyldermans et al, J. Mol. Recognit. 12:131-40, 1999; Arbabi Ghahroudi et al., FEBS Lett. 414:521-26, 1997; Muyldermans et al., Protein Eng. 7:1129-35, 1994; Hamers-Casterman et al., Nature 363:446-48, 1993; the disclosures of which are incorporated by reference herein.) The variable region of heavy chain antibodies are typically referred to as “VHH” regions. (See, e.g., Muyldermans et al., TIBS 26:230-35, 2001.) The VHH of heavy chain antibodies typically have enlarged or altered CDR regions, as such enlarged CDR1 and/or CDR3 regions. Methods of producing heavy chain antibodies are also known in the art. (See, e.g., Arbabi Ghahroudi et al., supra; Muyldermans et al., supra.)

Antibody which contains the idiotype of the molecule can be generated by known techniques. For example, such fragments include but are not limited to, the F(ab′)2 fragment which can be produced by pepsin digestion of the antibody molecule, the Fab′ fragments which can be generated by reducing the disulfide bridges of the F(ab′)2 fragment, the Fab fragments which can be generated by treating the antibody molecule with papain and a reducing agent, and Fv fragments. Recombinant Fv fragments can also be produced in eukaryotic cells using, for example, the methods described in U.S. Pat. No. 5,965,405.

In the production of antibodies, screening for the desired antibody can be accomplished by techniques known in the art (e.g., ELISA (enzyme-linked immunosorbent assay)). In one example, antibodies which recognize a specific domain of a Retsat polypeptide can be used to assay generated hybridomas for a product which binds to a Retsat fragment containing that domain. For selection of an antibody that specifically binds to a first Retsat polypeptide derivative, but which does not specifically bind a different Retsat polypeptide, one can select on the basis of antibody positive binding to the first Retsat polypeptide and a lack of antibody binding to the second different Retsat polypeptide.

Antibodies specific to a domain of Retsat polypeptides are also provided. The foregoing antibodies can be used in methods known in the art relating to the localization and activity of the Retsat polypeptide sequences of the invention (e.g., for imaging proteins, measuring levels thereof in appropriate physiological samples, in diagnostic methods, and the like). In another embodiment of the invention (see infra), anti-Retsat antibodies and fragments thereof containing the antigen-binding domain are used as agents and compositions to slow, abate or alter cell proliferation, affect (e.g., increase or decrease or alter) retinoid metabolism, skin and/or immune function and regulation.

### Functional Assays for Retsat Polypeptides Fragments, Derivatives, and Analogs

The functional activity of Retsat polypeptides, fragments, derivatives and analogs can be assayed by various methods. For example, when assaying for the ability to bind or compete with wild-type Retsat polypeptide for binding to anti-Retsat antibody, various immunoassays known in the art can be used. Such assays include, but are not limited to, competitive and non-competitive assay systems using techniques such as radioimmunoassays, ELISA (enzyme linked immunosorbent assay) “sandwich” immunoassays, immunoradiometric assays, gel diffusion precipitin reactions, immunodiffusion assays, in situ immunoassays (using colloidal gold, enzyme or radioisotope labels, and the like), Western blots, precipitation reactions, agglutination assays (e.g., gel agglutination assays, hemagglutination assays), complement fixation assays, immunofluorescence assays, protein A assays, immunoelectrophoresis assays, and the like. (See generally Harlow and Lane, supra). Antibody binding can be detected by measuring a label on the primary antibody. Alternatively, the primary antibody is detected by measuring binding of a secondary antibody or reagent to the primary antibody. The secondary antibody can also be directly labeled. Many means are known in the art for detecting binding in an immunoassay and are considered within the scope of the present invention.

In another embodiment, the ability of Retsat polypeptide to convert all-trans-retinol to all-trans-(13,14)-dihydroretinol (infra).

### In Vivo Uses of RETSAT Nucleic Acids, Retsat Polypeptides, Fragments, Derivatives, Analogs and Antibodies

The invention provides further for methods for the administration of one or more agents, or compositions containing such agents, which modulate cell proliferation, retinoid metabolism, skin and/or immune function and regulation. Such agents include, but are not limited to, Retsat polypeptides, fragments, derivatives and analogs thereof as described hereinabove; antibodies specific for Retsat polypeptide, fragments, derivatives and analogs thereof (as described hereinabove); all-trans-(13,14)-dihydroretinol, all all-trans-(13,14)-dihydroretinoic acid, all-trans-(13,14)-dihydroretinoid derivatives, and Retsat polypeptide agonists and antagonists. The Retsat agents can be used to treat disorders involving cancer, blindness, skin and immunological disorders by altering Retsat function.

Generally, it is typical to administer an agent of a species origin or species reactivity (in the case of antibodies) that is the same as that of the recipient. Thus, a human Retsat polypeptide, fragment, derivative, or analog thereof, or RETSAT nucleic acid or fragment or analog thereof, or an antibody to a human Retsat polypeptide, is administered to a human in a dose which is therapeutically or prophylactically effective.

Diseases involving cancer, blindness, skin diseases or conditions and/or immunological disorders. Examples of such an agent include, but are not limited to, anti-sense RETSAT nucleic acids under the control of a strong inducible promoter, particularly those that are active in liver, kidney, and intestine. Other agents that can be used to decrease Retsat activity include anti-Retsat antibodies, or those that can be identified using in vitro assays or animal models, examples of which are described herein.

In specific embodiments, agents that decrease RETSAT function are administered therapeutically (including prophylactically) in diseases involving an increased (relative to normal or desired) level of Retsat polypeptide or function. For example, the agent can be administered to a patient where Retsat polypeptide is overexpressed, genetically defective, or biologically hyperactive, as compared with a normal cell of that type. Further, an agent of the invention can be administered in diseases or disorders wherein in vitro (or in vivo) assays indicate the utility of Retsat antagonist administration.

The level in Retsat polypeptide or function can be detected, for example, by obtaining a patient tissue sample (such as from a biopsy tissue) and assaying it in vitro for RNA or polypeptide levels, structure and/or activity of the expressed RETSAT RNA or Retsat polypeptide. Many methods standard in the art can be thus employed including, but not limited to, immunoassays to detect and/or visualize Retsat polypeptide (e.g., Western blot, immunoprecipitation followed by sodium dodecyl sulfate polyacrylamide gel electrophoresis (“SDS PAGE”), immunocytochemistry, and the like) and/or hybridization assays to detect RETSAT expression by detecting and/or visualizing RETSAT mRNA (e.g., Northern blot assays, dot blots, in situ hybridization, quantitative reverse transcriptase-PCR, and the like), among others known to the skilled artisan.

Diseases involving cancer, blindness, skin conditions and disorders and immunological disorders that can be treated or prevented include, but are not limited to, acute promyelocytic leukemia, dermatological disorders, and the like.

Compositions of the invention, including an effective amount of all-trans-(13,14)-dihydroretinol in a pharmaceutically acceptable carrier, can be administered to a patient. The amount all-trans-(13,14)-dihydroretinol which will be effective in the treatment of a particular disease will depend on the nature of the disease, and can be determined by standard clinical techniques.

In an exemplary embodiment, compositions comprising all-trans-(13,14)-dihydroretinol, all-trans-13,14-dihydroretinoic acid and 13,14-dihydroretinoid derivatives and a pharmaceutically acceptable carrier are administered.

An agent can be administered to human or other non-human vertebrates. In certain embodiments, the agent is administered to an aging human. In certain embodiments, the agent is substantially pure, in that is contains less than about 5% or less than about 1%, or less than about 0.1%, other retinoids. In other embodiments, a combination of agents can be administered.

Agents can be delivered to the eye by any suitable means, including, for example, oral or local administration. Modes of local administration can include, for example, eye drops, intraocular injection or periocular injection. Periocular injection typically involves injection of the agents into the conjunctiva or to the tennon (the fibrous tissue overlying the eye). Intraocular injection typically involves injection of the agent into the vitreous. In certain embodiments, the administration is non-invasive, such as by eye drops or oral dosage form.

Agents can be formulated for administration using pharmaceutically acceptable vehicles as well as techniques routinely used in the art. A vehicle is selected according to the solubility of the agent. Suitable ophthalmological compositions include those that are administrable locally to the eye, such as by eye drops, injection or the like. In the case of eye drops, the formulation can also optionally include, for example, ophthalmologically compatible agents such as isotonizing agents such as sodium chloride, concentrated glycerin, and the like; buffering agents such as sodium phosphate, sodium acetate, and the like; surfactants such as polyoxyethylene sorbitan mono-oleate (also referred to as Polysorbate 80), polyoxyl stearate 40, polyoxyethylene hydrogenated castor oil, and the like; stabilization agents such as sodium citrate, sodium edentate, and the like; preservatives such as benzalkonium chloride, parabens, and the like; and other ingredients. Preservatives can be employed, for example, at a level of from about 0.001 to about 1.0% weight/volume. The pH of the formulation is usually within the range acceptable to ophthalmologic formulations, such as within the range of about pH 4 to 8.

For injection at or in the eye, the agent can be provided in an injection grade saline solution, in the form of an injectable liposome solution, or the like. Intraocular and periocular injections are known to those skilled in the art and are described in numerous publications including, for example, Ophthalmic Surgery: Principles of Practice, Ed., G. L. Spaeth, W. B. Sanders Co., Philadelphia, Pa., U.S.A., pages 85-87, 1990.

Suitable oral dosage forms include, for example, tablets, pills, sachets, or capsules of hard or soft gelatin, methylcellulose or of another suitable material easily dissolved in the digestive tract. Suitable nontoxic solid carriers can be used which include, for example, pharmaceutical grades of mannitol, lactose, starch, magnesium stearate, sodium saccharin, talcum, cellulose, glucose, sucrose, magnesium carbonate, and the like. (See, e.g., Remington “Pharmaceutical Sciences”, 17 Ed., Gennaro (ed.), Mack Publishing Co., Easton, Pa., 1985.)

The doses of the agents can be suitably selected depending on the clinical status, condition and age of the subject, dosage form and the like. In the case of eye drops, a agent can be administered, for example, from about 0.01 mg, about 0.1 mg, or about 1 mg, to about 25 mg, to about 50 mg, to about 90 mg per single dose. Eye drops can be administered one or more times per day, as needed. In the case of injections, suitable doses can be, for example, about 0.0001 mg, about 0.001 mg, about 0.01 mg, or about 0.1 mg to about 10 mg, to about 25 mg, to about 50 mg, or to about 90 mg of the agent, one to four times per week. In other embodiments, about 1.0 to about 30 mg of agent can be administered one to three times per week.

Oral doses can typically range from about 1.0 to about 1000 mg, one to four times, or more, per day. An exemplary dosing range for oral administration is from about 10 to about 250 mg one to three times per day.

In various embodiments of the invention, it can be useful to use such compositions to achieve sustained release of all-trans-(13,14)-dihydroretinol.

Diseases involving cancer, blindness, skin conditions and disorders and immunological disorders are also treated or prevented by administration all-trans-(13,14)-dihydroretinol, all-trans-13,14-dihydroretinoic acid and/or 13,14-dihydroretinoid derivatives.

The invention provides methods for the administration to a subject of an effective amount of all-trans-(13,14)-dihydroretinol, all-trans-13,14-dihydroretinoic acid and/or 13,14-dihydroretinoid derivatives. Typically, the all-trans-(13,14)-dihydroretinol, all-trans-13,14-dihydroretinoic acid and/or 13,14-dihydroretinoid derivatives is substantially purified prior to formulation. The subject or patient can be an animal, including but not limited to, cows, pigs, horses, chickens, cats, dogs, and the like, and is typically a mammal, and in a particular embodiment human. In another specific embodiment, a non-human mammal is the subject.

Methods of introduction include but are not limited to intradermal, intramuscular, intraperitoneal, intravenous, subcutaneous, intranasal, intraocular, epidural and oral routes. The agents can be administered by any convenient route such as, for example, by infusion or bolus injection, by absorption through epithelial or mucocutaneous linings (e.g., oral mucosa, rectal and intestinal mucosa), and the like, and can be administered together with other functionally active agents. Administration can be systemic or local. In addition, it can be desirable to introduce all-trans-(13,14)-dihydroretinol into the target tissue by any suitable route, including intravenous and intrathecal injection.

In a specific embodiment, it can be desirable to administer the agent locally to the area in need of treatment; this administration can be achieved by, for example, and not by way of limitation, local infusion during surgery, topical application, by injection (e.g., intraocular injection), by means of a catheter, by means of a suppository, or by means of an implant, the implant being of a porous, non-porous, or gelatinous material, including membranes such as sialastic membranes, or fibers.

In yet another embodiment, the agent can be delivered in a controlled release system. In one embodiment, a pump can be used (see, e.g., Langer, supra; Sefton, Crit. Ref. Biomed. Eng. 14:201-40, 1987; Buchwald et al., Surgery 88:507-16, 1980; Saudek et al., N. Engl. J. Med. 321:574-79, 1989). In another embodiment, polymeric materials can be used (see Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, N.Y., 1984; Ranger and Peppas, J. Macromol. Sci. Rev. Macromol. Chem. 23:61, 1983; see also Levy et al., Science 228:190-92, 1985; During et al., Ann. Neurol. 25:351-56, 1989; Howard et al., J. Neurosurg. 71:105-12, 1989). In yet another embodiment, a controlled release system can be placed in proximity of the therapeutic target, thus requiring only a fraction of the systemic dose (see, e.g., Goodson, in Medical Applications of Controlled Release, supra, Vol. 2, pp. 115-38, 1984). Other controlled release systems are discussed in, for example, the review by Langer, Science 249:1527-33, 1990.

The present invention also provides pharmaceutical compositions. Such compositions comprise a therapeutically effective amount of all-trans-(13,14)-dihydroretinol, all-trans-13,14-dihydroretinoic acid and/or 13,14-dihydroretinoid derivatives, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more typically in humans. The term “carrier” refers to a diluent, adjuvant, excipient, stabilizer, or vehicle with which the agent is formulated for administration. Pharmaceutical carriers can be sterile liquids, such as water and oils, including those of petroleum, animal, vegetable or synthetic origin, such as peanut oil, soybean oil, mineral oil, sesame oil, and the like. Water is a typical carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Pharmaceutical compositions can take the form of solutions, suspensions, emulsion, tablets, pills, capsules, powders, sustained-release formulations, and the like. The composition can be formulated as a suppository, with traditional binders and carriers such as triglycerides.

Oral formulations can include standard carriers such as pharmaceutical grades of mannitol, lactose, starch, magnesium stearate, sodium saccharine, cellulose, magnesium carbonate, and the like. In an exemplary embodiment, an oral formulation comprising all-transs-(13,14)-dihydroretinol is formulated as a vitamin. Examples of suitable pharmaceutical carriers are described in, for example, Remington's Pharmaceutical Sciences (Gennaro (ed.), Mack Publishing Co., Easton, Pa., 1990). Such compositions will contain a therapeutically effective amount of all-trans-(13,14)-dihydroretinol, typically in purified form, together with a suitable amount of carrier so as to provide a formulation proper for administration to the patient. The formulation should suit the mode of administration.

In one embodiment, the agent is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent. Generally, the ingredients are supplied either separately or mixed together in unit dosage form. For example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The agents of the invention can be formulated as neutral or salt forms. Pharmaceutically acceptable salts include those formed with free amino groups such as those derived from hydrochloric, phosphoric, acetic, oxalic, tartaric acids, and the like, and those formed with free carboxyl groups such as those derived from sodium, potassium, ammonium, calcium, ferric hydroxides, isopropylamine, triethylamine, 2-ethylamino ethanol, histidine, procaine, and the like.

The amount of the agent which will be effective in the treatment of a particular disease or condition. In addition, in vitro assays can optionally be employed to help identify optimal dosage ranges. The precise dose of the agent to be employed in the formulation will also depend on the route of administration, and the seriousness of the disease, and should be decided according to the judgment of the practitioner and each patient's circumstances. Suitable dosage ranges for intravenous administration are generally about 20-500 micrograms of active agent per kilogram body weight. Suitable dosage ranges for intranasal administration are generally about 0.01 pg/kg body weight to 1 mg/kg body weight. Effective doses can be extrapolated from dose response curves derived from in vitro or animal model test systems. Oral formulations typically contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Screening for Agonists and Antagonists

RETSAT nucleic acids, Retsat polypeptide, and fragments, derivatives and analogs thereof, also have uses in screening assays to detect candidate compounds that specifically bind to RETSAT nucleic acids, Retsat polypeptides, or fragments, derivatives or analogs thereof, and thus have use as agonists or antagonists. The agonists and antagonists can be identified by in vitro and/or in vivo assays. Such assays can be used to identify agents that are therapeutically effective, or as lead compounds for drug development. The invention thus provides assays to detect candidate compounds that specifically affect the activity or expression of RETSAT nucleic acids, Retsat polypeptides, or fragments, derivatives or analogs thereof.

In a typical in vivo assay, recombinant cells expressing RETSAT nucleic acids can be used to screen candidate compounds for those that affect RETSAT expression. Effects on RETSAT expression can include the synthesis or levels of all-trans-13,14-dihydroretinoic acid and/or 13,14-dihydroretinoid derivatives.

Candidate compounds can also be identified by in vitro screens. For example, recombinant cells expressing RETSAT nucleic acids can be used to recombinantly produce Retsat polypeptide for in vitro assays to identify candidate compounds that bind to Retsat polypeptide. Candidate compounds (such as putative binding partners of Retsat or small molecules) are contacted with the Retsat polypeptide (or fragment, derivative or analog thereof) under conditions conducive to binding, and then candidate compounds that specifically bind to the Retsat polypeptide are identified. Similar methods can be used to screen for candidate compounds that bind to nucleic acids encoding RETSAT, or a fragment, derivative or analog thereof. Methods that can be used to carry out the foregoing are commonly known in the art, and include diversity libraries, such as random or combinatorial peptide or non-peptide libraries that can be screened for candidate compounds that specifically bind to Retsat polypeptide. Many libraries are known in the art, such as, for example, chemically synthesized libraries, recombinant phage display libraries, and in vitro translation-based libraries.

Examples of chemically synthesized libraries are described in Fodor et al., Science 251:767-73, 1991, Houghten et al. Nature 354:84-86, 1991, Lam et al. Nature 354:82-84, 1991, Medynski, Bio/Technology 12:709-10, 1994, Gallop et al. J. Med. Chem. 37:1233-51, 1994, Ohlmeyer et al. Proc. Natl. Acad. Sci. USA 90:10922-26, 1993, Erb et al., Proc. Natl. Acad. Sci. USA 91:11422-26, 1994, Houghten et al. Biotechniques 13:412-21, 1992, Jayawickreme et al. Proc. Natl. Acad. Sci. USA 91:1614-18, 1994, Salmon et al. Proc. Natl. Acad. Sci. USA 90:11708-12, 1993, International Patent Publication WO 93/20242, and Brenner and Lemer, Proc. Natl. Acad. Sci. USA 89:5381-83, 1992.

Examples of phage display libraries are described in Scott and Smith, Science 249:386-90, 1990, Devlin et al. Science 249:404-06, 1990, Christian et al. J. Mol. Biol. 227:711-18, 1992, Lenstra, J. Immunol. Meth. 152:149-57, 1992, Kay et al. Gene 128:59-65, 1993, and International Patent Publication WO 94/18318.

In vitro translation-based libraries include, but are not limited to, those described in International Patent Publication WO 91/05058, and Mattheakis et al. Proc. Natl. Acad. Sci. USA 91:9022-26, 1994. By way of examples of nonpeptide libraries, a benzodiazepine library (see, e.g., Bunin et al., Proc. Natl. Acad. Sci. USA 91:4708-12, 1994) can be adapted for use. Peptide libraries (see, e.g., Simon et al., Proc. Natl. Acad. Sci. USA 89:9367-71, 1992) can also be used. Another example of a library that can be used, in which the amide functionalities in peptides have been permethylated to generate a chemically transformed combinatorial library, is described by Ostresh et al. Proc. Natl. Acad. Sci. USA 91:11138-42, 1994).

Screening of the libraries can be accomplished by any of a variety of commonly known methods. See, for example, the following references, which disclose screening of peptide libraries: Parmley and Smith, Adv. Exp. Med. Biol. 251:215-18, 1989; Scott and Smith (supra); Fowlkes et al. BioTechniques 13:422-28, 1992; Oldenburg et al. Proc. Natl. Acad. Sci. USA 89:5393-97, 1992; Yu et al. Cell 76:933-45, 1994; Staudt et al. Science 241:577-80, 1988; Bock et al. Nature 355:564-66, 1992; Tuerk et al. Proc. Natl. Acad. Sci. USA 89:6988-92, 1992; Ellington et al. Nature 355:850-52, 1992; U.S. Pat. Nos. 5,096,815, 5,223,409, and 5,198,346, all to Ladner et al.; Rebar and Pabo, Science 263:671-73, 1994; and International Patent Publication WO 94/18318.

In a specific embodiment, screening can be carried out by contacting the library members with a Retsat polypeptide (or nucleic acid or derivative) immobilized on a solid phase and harvesting those library members that bind to the polypeptide (or nucleic acid or derivative). Examples of such screening methods, termed “panning” techniques are described by way of example in Parmley and Smith, Gene 73:305-18, 1988; Fowlkes et al. (supra); International Patent Publication WO 94/18318; and in references cited hereinabove.

### Animal Models

The invention also provides animal models. In one embodiment, animal models for diseases involving cancer, blindness, skin conditions and disorders and immunological disorders are provided. Such an animal can be initially produced by promoting homologous recombination between a RETSAT gene in its chromosome and an exogenous RETSAT nucleic acid that has been rendered biologically inactive (typically by insertion of a heterologous sequence, such as an antibiotic resistance gene). In one aspect, homologous recombination is carried out by transforming embryo-derived stem (ES) cells with a vector containing the insertionally inactivated RETSAT gene, such that homologous recombination occurs, followed by injecting the ES cells into a blastocyst, and implanting the blastocyst into a foster mother, followed by the birth of the chimeric animal (“knockout animal”) in which a RETSAT gene has been inactivated (see Capecchi, Science 244:1288-92 (1989)). The chimeric animal can be bred to produce additional knockout animals. Such animals can be mice, rats, hamsters, sheep, pigs, cattle, and the like, and are typically non-human mammals. In a specific embodiment, a knockout mouse is produced. Knockout animals are expected to develop, or be predisposed to developing diseases, involving cancer, blindness, skin conditions and disorders and immunological disorders and can be useful to screen for or test candidate compounds.

In a different embodiment of the invention, transgenic animals that have incorporated and express a functional RETSAT gene have use as animal models of diseases involving cancer, blindness, skin conditions and disorders and immunological disorders. Transgenic animals are expected to develop or be predisposed to developing diseases involving cancer, blindness, skin conditions and disorders and immunological disorders and thus can have use as animal models of such diseases (e.g., to screen for or test candidate compounds.

The following examples are provided merely as illustrative of various aspects of the invention and shall not be construed to limit the invention in any way.

### Exemplary Embodiments

### EXAMPLE 1

### Retinoids Essential for Important Biological Functions

Retinoids are essential for many important biological functions, such as development, immunity, cellular differentiation, and vision of vertebrates. Retinoids encompassing both natural derivatives of all-trans-retinol and their synthetic analogues exert their functions through several active compounds. Esterification of retinol by lecithin-retinol acyltransferase (LRAT) leads to retinyl esters, which represent both a major storage form of vitamin A and an intermediate of the visual cycle. Ruiz, et al. J Biol Chem 274:3834-3841, 1999; Batten, et al. J Biol Chem 279:10422-10432, 2004; Imanishi, et al. J Cell Biol 164:373-383, 2004. In retinal pigment epithelium (RPE) an unidentified enzyme carries out the isomerization of all-trans-retinol either directly or through an ester intermediate to generate 11-cis-retinol, which can be oxidized to 11-cis-retinal, the visual chromophore. Kuksa, et al. Vision Res 43:2959-2981, 2003. Reversible oxidation to retinal can be carried out by several members of the microsomal, short-chain alcohol dehydrogenase family (SCAD) and possibly by class I, II, and IV medium-chain alcohol dehydrogenases (ADH). Chou, et al. J Biol Chem 277:25209-25216, 2002; Duester, et al. Chem Biol Interact 143-144, 201-210, 2003. Oxidation of retinal by retinal dehydrogenase (RALDH) types 1, 2, 3 and 4 generates retinoic acid (RA), which controls development and cellular differentiation via nuclear receptors. Bhat, et al. Gene 166:303-306, 1995; Penzes, et al. Gene 191:167-172, 1997; Wang, et al. J Biol Chem 271:16288-16293, 1996; Zhao, et al. Eur J Biochem 240:15-22, 1996; Mic, et al. Mech Dev 97:227-230, 2000; Lin, et al. J Biol Chem 278:9856-9861, 2003; Chambon, Faseb J 10:940-954, 1996. RA-inducible cytochrome P450 enzymes CYP26A1 and B1 carry out the catabolism of RA to polar 4-hydroxy-RA, 4-oxo-RA and 18-hydroxy-RA. Abu-Abed, et al. J Biol Chem 273:2409-2415, 1998; Fujii, et al. Embo J 16:4163-4173, 1997; White, et al. J Biol Chem 271:29922-29927, 1996; White, et al. Proc Natl Acad Sci USA 97:6403-6408, 2000. Specific localization of RA anabolizing and catabolizing enzymes are essential for embryonic patterning. Other pathways generate retro-retinoids such as 14-hydroxy-4,14-retro-retinol (14-HRR) and anhydroretinol (AR), whose opposing effects control cell growth. Buck, et al. Science 254:1654-1656, 1991; Buck, et al. J Exp Med 178:675-680, 1993. Given the low levels and labile nature of retinoids in biological systems, and the incompletely understood mechanism of their biotransformations, many of the enzymes involved in retinoid metabolism remain to be discovered.

In addition to dietary sources, retinoids are derived from the cleavage of C40 provitamin A carotenoids such as α- and β-carotene and cryptoxanthin to produce retinal, which can be converted to all-trans-retinol. Provitamin A carotenoids also represent a major storage form of retinoids in tissues, serum, and the vertebrate egg yolk. Only two enzymes involved in the metabolism of β-carotene in animals have been identified: β,β-carotene-15,15′-monooxygenase (BCO-I), which carries out the symmetric cleavage of β-carotene to produce retinal, and β,β-carotene-9′,10′-oxygenase (BCO-II), which carries out the asymmetric cleavage to generate β,β-ionone and β-apo-10′-carotenal. von Lintig, et al. Proc Natl Acad Sci USA 98:1130-1135, 2001; Kiefer, et al. J Biol Clem 276:14110-14116, 2001. BCO-I and II have sequence similarity to VP14, the 9-cis-neoxanthin cleavage enzyme from Zea mais, and other carotenoid cleavage enzymes from plants (reviewed in Giuliano, et al. Trends Plant Sci 8:145-149, 2003). BCO-I was first identified in flies based on its similarity to VP14 and later cloned from mice and humans. von Lintig, et al. Proc Natl Acad Sci USA 98:1130-1135, 2001; Redmond, et al. J Biol Chem 276:6560-6565, 2001; Yan, et al. Genomics 72:193-202, 2001. Other, more limited dietary sources of retinoids are all-trans-retinyl esters and free all-trans-retinol. In addition to β-carotene, retinal, and retinoic acid, animal tissues also retain considerable amounts of non-provitamin A carotenoids such as lutein and zeaxanthin in the primate macula, and lycopene in serum and most tissues. Non-provitamin A carotenoids as well as uncleaved β-carotene have been implicated in the prevention of cancer, macular degeneration, and heart disease (reviewed in Snodderly, Am J Clin Nutr 62:1448S-1461S, 1995 and Fraser, et al. Prog Lipid Res 43:228-265, 2004). Despite this interest the enzymes involved in the metabolism and physiology of carotenoids in animals await molecular identification.

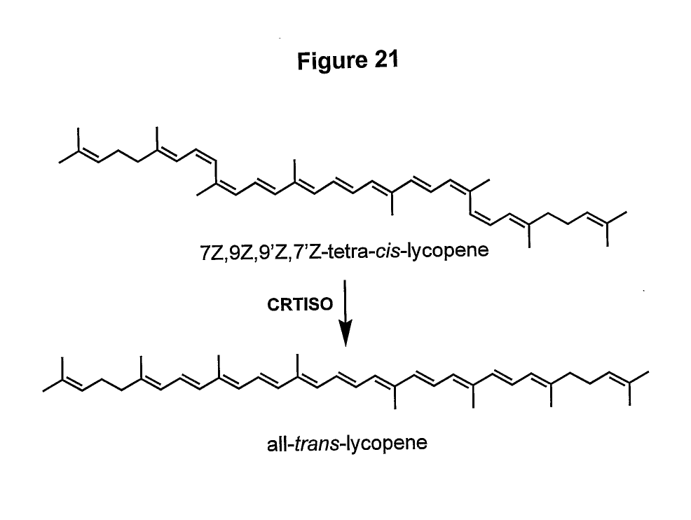

Powerful genetic approaches and readily identifiable phenotypes aided in the discovery of the biochemical pathways of carotenoid synthesis in plants and bacteria. These enzymes could serve as a model to uncover carotenoid or retinoid enzymes in vertebrates. Recently, the enzymes responsible for the isomerization of 7Z,9Z,9′Z,7′Z-tetra-cis-lycopene (also known as prolycopene) to all-trans-lycopene in plants and cyanobacterium Synechocystis (FIG. 21) have been characterized. Park, et al. Plant Cell 14:321-332, 2002; Isaacson, et al. Plant Cell 14:333-342, 2002; Breitenbach, et al. Z Naturforsch [C] 56:915-917, 2001; Masamoto, et al. Plant Cell Physiol 42:1398-1402, 2001; Giuliano, et al. Trends Plant Sci 7:427-429, 2002. FIG. 21 shows reaction catalyzed by plant and cyanobacterial CRTISO. Tomato CRTISO mutants are known for their tangerine phenotype due to accumulation of 7Z,9Z,9′Z,7′Z-tetra-cis-lycopene. Zechmeister, et al. Proc Natl Acad Sci USA 21:468-474, 1941 The protein sequences of CRTISO enzymes are similar to CrtI from non-photosynthetic bacteria, an enzyme that catalyzes the direct conversion of phytoene into all-trans-lycopene. Giuliano, et al. J Biol Chem 261:12925-12929, 1986. It also bears resemblance to phytoene desaturase, Pds, and ζ-carotene desaturase, or Zds, from plants, which each introduce two double bonds during the four desaturation steps from phytoene to 7Z,9Z,9′Z,7′Z-tetra-cis-lycopene (trans-H elimination at 11,11′ by Pds and cis-H elimination at 7,7′ by Zds) (reviewed in Sandmann, Physiologia Plantarum 116:431-440, 2002).

Searching for homologous proteins using the protein sequences of plant CRTISO, we found similarities in a few hypothetical proteins predicted by the conceptual translation of expressed sequence tags (ESTs) and transcripts from vertebrates and non-vertebrates. Expression of the mouse CRTISO-like protein in transfected cells revealed that it catalyzes saturation of all-trans-retinol at the 13-14 double bond, and the mouse CRTISO-like protein was thus designated RetSat. This is in contrast to tomato CRTISO, which catalyzes cis-trans isomerization of lycopene and has no saturase activity versus all-trans-retinol. moreover, the saturated product, all-trans-13,14-dihydroretinol, is detected in several tissues of animals maintained on a diet containing normal levels of vitamin A.

### EXAMPLE 2

### Materials And Methods

Cloning of mouse and monkey RetSat and tomato CRTISO and creation of stable cell lines with inducible expression. RPE was microdissected from a C57/BL6 mouse or macaque crab-eating monkey (Macaca fascicularis). RNA from mouse or monkey RPE and from ripe, red tomato was isolated using the MicroAqueous RNA Isolation Kit (Ambion, Austin, Tex.) and reverse-transcribed using SuperScript II Reverse Transcriptase (Invitrogen, Carlsbad, Calif.) and oligo(dT) primers according to manufacturer's protocol. Mouse RetSat cDNA was amplified using Hotstart Turbo Pfu Polymerase (Stratagene, La Jolla, Calif.) and the primers: 5′-ATGTGGATCACTGCTCTGCTGCTGG-3′ (forward) (SEQ ID NO: 1) and 5′-TCTGGCTCTTCTCTGAACGGACTACATC-3′ (reverse) (SEQ ID NO: 2); monkey RetSat was amplified with primers: 5′-CAGTCGGAGCTGTCCCATTTACC-3′ (forward) (SEQ ID NO: 3) and 5′-AAATTCCTCTGACTCCTCCCTGATG-3′ (reverse) (SEQ ID NO: 4); tomato CRTISO was amplified using the primers: 5′-CTTTCCAGGGAGCCCAAAAT-3′ (forward) (SEQ ID NO: 5) and 5′-ACATCTAGATATCATGCTAGTGTCCTT-3′ (reverse) (SEQ ID NO: 6). For expression of mouse RetSat, cDNA was amplified with the primers:

5′-CCTCTAGAGCCACCATGTGGATCACTGCTCTGCTGCTGG-3′ (forward) (SEQ ID NO: 7) and

5′-ACTAGTCTACATCTTCTTCTTTTGTGCCTTGACCTTTGA-3′ (reverse) (SEQ ID NO: 8) and cloned into the tetracycline-inducible, eukaryotic expression vector pCDNA4/TO (Invitrogen) using Xba I/Spe I, while tomato CRTISO was amplified using the primers

5′-TCTAGAAGGAGGACAGCAATGGTAGATGTAGACAAAAGAGTGGA-3′ (forward) (SEQ ID NO: 9) and 5′-ACATCTAGATATCATGCTAGTGTCCTT-3′ (reverse) (SEQ ID NO: 10) and cloned into the Xba I site of pCDNA4/TO. N-acetylglucosaminyltransferase I-negative HEK293S cells, obtained from Dr. G. Khorana (MIT, Boston, Mass.), were transfected with the tetR-expression plasmid pCDNA6-TR(blaR), and blasticidin-resistant colonies were selected and cloned. A stable tetR-expressing clone of HEK-293S designated HEK-Khorana (HEKK) was then transfected with pCDNA4/TO (zeoR) containing either mouse RetSat or tomato CRTISO cDNA and selected with zeocin. All zeocin-resistant clones were pooled and used in activity assays. Cells were cultured in DMEM, 10% fetal calf serum plus zeocin and blasticidin antibiotics and maintained at 37° C., 5% CO₂ and 100% humidity.

Inducible-Expression Of LRAT Protein in HEKK Cells. Mouse Lrat cDNA was cloned as described elsewhere. For expression Lrat coding region was amplified using the primers: 5′-GCCACCATGAAGAACCCAATGCTGGAAGCT-3′ (SEQ ID NO: 11) and ACATACACGTTGACCTGTGGACTG (SEQ ID NO: 12). The PCR product was ligated into the pCR-Blunt II-TOPO vector (Invitrogen) and then subcloned into the EcoRI site of pCDNA4/TO. TetR-expressing HEKK cells were transfected with the pCDNA4/TO-Lrat construct and selected with zeocin. Stable clones were verified for expression of Lrat protein using the anti-LRAT monoclonal antibody described elsewhere (2).

Purification of a bacterially expressed His-tagged mouse RetSat fragment, polyclonal and monoclonal antibody production. The Nco I fragment of RetSat cDNA, corresponding to nucleotide 440-1391 of mouse RetSat cDNA (coding for ¹⁴⁹MASPF . . . MTALVPM⁴⁶⁵ polypeptide fragment) was cloned into the Nco I site of the inducible bacterial expression vector pET30B (Invitrogen). This resulted in a recombinant protein tagged at both amino and carboxyl termini with hexa-histidine tags. The plasmid was transformed into BL-21RP cells (Stratagene) and expression was induced with IPTG. The double (His)₆-tagged fragment of the mouse RetSat protein (40 kDa) was purified by Ni-NTA affinity using manufacturer's protocol (Qiagen, Valencia, Calif.). The purified protein was examined by gel electrophoresis. Following in-gel trypsin digestion the eluted tryptic peptides were examined by microsequencing by LC/MS to verify the identity of the recombinant RetSat fragment. The purified protein was used to immunize mice as described before and the monoclonal antibody produced by established methods. Haeseleer, et al. J Biol Chem 277:45537-45546, 2002; Adamus, et al. In Vitro Cell Dev Biol 25:1141-1146, 1989. Rabbit polyclonal antiserum was raised in collaboration with Cocalico Biologicals Inc. (Reamstown, Pa.). The sera and monoclonal antibody were tested for their specificity by immunocytochemistry and immunoblotting of RetSat-transfected versus untransfected cells. Anti-RetSat IgG was purified from the ascitic supernatant of RetSat-producing hybridoma cells using a HiTRAP protein G HP (Amersham, Piscataway, N.J.) using the manufacturer's protocol. The purified antibody was coupled with fluorophore using the Alexa Fluor 488 monoclonal antibody coupling kit (Invitrogen) following the manufacturer's protocol.

Northern blot analysis of mouse RetSat transcripts. Northern blot analysis was performed using a commercially available premade blot containing 2 μg poly (A) RNA from various mouse tissues per lane (FirstChoice Northern Blot Mouse Blot I, Ambion) following the manufacturer's protocol. The [α³²P]-radiolabeled probe was constructed by run-off PCR of mouse RetSat cDNA using the 5′-TCTGGCTCTTCTCTGAACGGACTACATC-3′ reverse primer and the Strip-EZ probe synthesis kit from Ambion following the manufacturer's protocol. Alternatively, a radiolabeled antisense mouse beta-actin probe was constructed using the T7 primer and the pTRIamp 18 βP-actin template (Ambion).

Immunoblotting and immunohistochemistry analysis of mouse RetSat. To establish the membrane association of RetSat, mouse liver was homogenized in 50 mM Tris-HCl, pH 8.0, containing 250 mM sucrose, 5 mM dithiothreitol, and 1× protease inhibitor cocktail (Sigma-Aldrich, St. Louis, Mo.) using a douncer. The nuclei and extracellular matrix were pelleted by centrifugation for 30 min at 20,000 g and discarded. The high-speed cytosolic supernatant and post-nuclear membranes were separated by centrifugation at 145,000 g for 90 min. Post-nuclear membranes were homogenized in 10 mM Tris, pH 8.0, containing 200 mM NaCl, 1 mM EDTA, 1% Triton X-100, and 10 μM PMSF. The protein concentration was measured in whole cell lysate, high-speed cytosolic supernatant and post-nuclear membrane fraction using the Bradford assay. Bradford, Anal Biochem 72:248-254, 1976. Equal amounts of protein were resolved on SDS-PAGE and stained with 1/1000 dilution of anti-RetSat monoclonal antibody and 1/10⁴ goat anti-mouse IgG (Fc) (Promega, Madison, Wis.). To examine tissue-specific expression of RetSat, various mouse tissues were dissected and homogenized with 10 mM Tris, pH 8.0, containing 10 mM 2-mercaptoethanol and 10 μM PMSF, with the aid of a douncer. The membranes were pelleted by centrifugation at 12,000 g for 30 min. The protein concentration was measured using the Bradford assay. Bradford, Anal Biochem 72:248-254, 1976. Equal amounts of protein (10 μg) from the membrane fraction of each tissue were resolved by SDS-PAGE and stained by immunoblotting with 1/1000 dilution of rabbit anti-RetSat polyclonal antiserum and alkaline phosphatase-coupled 1/10⁴ goat anti-rabbit IgG (Fc) (Promega) secondary antibody. The mouse monoclonal anti-RetSat showed the same reactivity in the examined tissues as the polyclonal antiserum. Untransfected HEKK or HEKK-RetSat cells were cultured in Dulbecco's modified Eagle's medium (Invitrogen) on glass bottom microwell dishes (MatTek Corp., Ashland, Mass.). Expression of RetSat was induced by the addition of 1 μg/ml tetracycline. Cells were harvested after 48 hr and fixed with 4% paraformaldehyde (Fisher, Hampton, N.H.) in PBS (136 mM NaCl, 11.4 mM sodium phosphate, pH 7.4) for 10 min and washed by PBS. To block nonspecific labeling, the cells were incubated in 1.5% normal goat serum (Vector Lab., Inc., Burlingame, Calif.) in PBST (136 mM NaCl, 11.4 mM sodium phosphate, 0.1% Triton X-100, pH 7.4) for 15 min at room temperature. The cells were incubated overnight at 4° C. in Alexa 488-coupled anti-RetSat monoclonal IgG diluted with PBST. The sections were rinsed in PBST and mounted in 50 μl of 2% 1,4-diazabicyclo-[2.2.2]octane (Sigma-Aldrich) in 90% glycerol to retard photobleaching. For confocal imaging, the cells were analyzed on a Zeiss LSM510 laser-scanning microscope (Carl Zeiss, Inc., Thornwood, N.Y.).

Retinol isomer purification and HPLC analysis of retinoids. All procedures involving retinoids were performed under dim red light unless otherwise specified. Retinoids were stored in N,N-dimethylformamide under argon at −80° C. All retinol and retinal substrates were purified by normal-phase HPLC (Beckman Ultrasphere-Si, 5 μm, 4.6 mm×250 mm, Fullerton, Calif.) with 10% ethyl acetate/90% hexane at a flow rate of 1.4 mmin using an HP1100 HPLC with a diode-array detector and HP Chemstation A. 08.03 software. For all-trans-13,14-dihydroretinol we used an extinction coefficient ε=16,500 at 290 nm. The following extinction coefficients were used for retinoids (in M−1·cm−1): all-trans-retinol, ε=51,770 at 325 nm; 9-cis-retinol, ε=42,300 at 323 nm; 11-cis-retinol, ε=34,320 at 318 nm; 13-cis-retinol, ε=48,305 at 328 nm; all-trans-retinal, ε=48,000 at 368 nm in hexane; and all-trans-retinoic acid, ε=45,300 at 350 nm in ethanol. Garwin, et al. Methods Enzymol 316:313-324, 2000. Retinoic acid was dissolved in ethanol and examined by reverse-phase HPLC System II (Zorbax ODS, 5 μm, 4.6 mm×250 mm, Agilent, Foster City, Calif.) with an isocratic mobile phase of 70% acetonitrile, 29% water, 1% glacial acetic acid, flow rate of 1.4 ml/min.

7Z,9Z,9′Z,7′Z-Tetra-cis-tycopene purification and reverse-phase HPLC analysis of carotenoids. Carotenoid extraction and analysis were performed under dim red light. One gram from the fruit of a tangerine tomato was freeze-thawed three times and extracted with 2 ml PBS, 2 ml of ethanol, and 6 ml of hexane with the aid of a douncer. The organic phase was dried and resuspended in ethanol/tetrahydrofuran (9:1), and examined by reverse-phase HPLC System I (Prontosil, 200-3-C30, 3 μm, 4.6 mm×250 mm, Bischoff Chromatography, Leonberg, Germany) with a mobile phase of 75% tert-butyl methyl ether/25% methanol and a flow rate of 1 ml/min. More than 90% of the extract consisted of 7Z,9Z,9′Z,7′Z-tetra-cis-lycopene identified based on its published UV/VIS absorption spectrum in hexane with the following characteristics: shoulder at λ=417 nm, ε=90,000 M⁻¹cm⁻¹, λₘₐₓ=437 nm, ε=105,000 M−1 cm⁻¹, shoulder at λ=461 nm ε=70,000 M⁻¹cm⁻¹. Bradford, Anal Biochem 72:248-254, 1976.

Enzyme assays of RetSat and CRTISO-catalyzed reactions. For enzyme assay, cells were seeded in six-well plates and expression of RetSat or CRTISO was induced with 1 μg/ml tetracycline 48 hr prior to analysis. Substrate preparation and addition were conducted under dim red light. Retinoid substrates were purified by HPLC as described above and dissolved in N,N-dimethylformamide (DMF) to a final concentration of 4 mM. Organic extract of tangerine tomatoes was dried under a stream of argon and resuspended in DMF. The substrates were diluted in 300 μl complete media (tetracycline 1 μg/ml) to a 40 μM final concentration, overlayed on cells, and incubated overnight in the dark at 37° C. in 5% CO₂ and 100% humidity. Media and cells were collected by scraping, mixed with an equal volume of methanol. For retinol and dihydroretinol analysis the methanol:water mixture was extracted with two volumes of hexane, then the organic phase was dried, resuspended in hexane, and analyzed by normal-phase HPLC. Retinal and dihydroretinal analysis was performed by treatment of the methanol:water mixture with 12.5 mM hydroxylamine followed by organic extraction and normal-phase HPLC. For carotenoid analysis the organic phase was dried and resuspended in ethanol/tetrahydrofuran (9:1) and examined by reverse-phase HPLC System I. For retinoic acid analysis the 1:1 methanol:water mixture was acidified with 0.1 volumes of 12N HCl and extracted with 1 volume of chlorofom, dried and resuspended in ethanol and examined by reverse-phase HPLC System II.

Chemical synthesis of all-trans-13,14-dihydroretinol. All reagents were purchased from Sigma-Aldrich or Fluka and were used without additional purification. Solvents were dried under standard procedures prior to use. All operations with retinoids were performed under dim red light unless otherwise specified. β-Ionone was condensed with triethyl phosphonoacetate in anhydrous THF in the presence of NaH to give ethyl trans-β-ionylideneacetate. This ester was then reduced with LiAlH4 to alcohol and reacted overnight with triphenylphosphine hydrobromide to give Wittig salt. Ethyl 4-oxo-3-methylcrotonate was hydrogenated in methanol with H2 using 10% Pd on C as a catalyst to yield ethyl 4-oxo-3-methylbutyrate, which was then reacted with Wittig salt using t-BuOK as a base in anhydrous CH₂Cl₂ in the presence of 18-crown-6. The obtained mixture of ethyl 11-cis- and all-trans-13,14-dihydroretinoates was reduced with LiAlH4 to 13,14-dihydroretinols, and all-trans-isomer was separated from 11-cis- by flash chromatography of silica gel using 5% ethyl acetate in hexane. NMR data was recorded on a Bruker 500 MHz spectrometer using CDCl₃ as an internal standard. 1H-NMR analysis of synthetic all-trans-13,14-dihydroretinol: NMR (CDCl₃, δ, ppm) 6.41 (dd, 1H, H-12, J 11.3, 14.75 Hz), 6.10-6.12 (m, 3H, H-7, H-8, H-10 J 15.7 Hz), 5.6 (dd, 1H, H-11, J 8.34, 14.95 Hz), 3.67 (m, 2H, CH₂-15), 2.41 (m, 1H, H-13, J 6.7 Hz), 1.96 (m, 2H, CH₂-14), 1.90 (s, 3H, CH₃-9), 1.69 (s, 3H, CH₃-5), 1.46 (m, 2H, CH₂-2), 1.6 (m, 4H, CH₂-3, CH₂-4), 1.06 (d, 3H, CH₃-13 J 6.7 Hz), 1.00 (s, 6H, 2×CH₃-1).

Isomerization and EI-MS analysis of all-trans-13,14-dihydroretinol Equal amounts (by UV absorbance) of the synthetic and biosynthetic compounds were resuspended in ethanol and exposed to sunlight for 30 min, followed by the addition of an equal volume of water and two volumes of hexane. The compounds were extracted, the organic phase was dried and resuspended in hexane, and the isomeric mixture was examined by normal-phase HPLC. For MS analysis the unknown biosynthetic metabolite and the chemically synthesized all-trans-13,14-dihydroretinol were purified by normal-phase HPLC and analyzed by EI-MS analysis using a JEOL HX-110 direct probe mass spectrometer. Some of the ions in the fragmentation patterns of both samples were 288 [M]+, 273 [M—CH₃]+, 243 [M—CH₂CH₂OH]+, 215 [M—CH(Me)(CH₂)₂OH]+, 202, 187, 159. The spectra are shown without manipulation.

Enzymatic assays for saturase activity in homogenized cells. Cells were homogenized with 15 mM Tris-HCl, pH 8.0, containing 10 mM dithiothreitol and 0.32 M sucrose. One aliquot of cells was boiled for 10 min at 95° C. as a negative control. Cell aliquots of 200 μl were supplemented with 1 mM ATP and 40 μM all-trans-retinol final concentrations. Some aliquots were also supplemented with 0.4 mM NADH or 0.4 mM NADPH to regenerate the redox state of the reaction. The cell homogenate was incubated with retinol substrate with shaking at 37° C. for 1 hr in the dark. This was followed by the extraction of retinoids with one volume of methanol and two volumes of hexane. The organic phase was dried and resuspended in hexane and then examined by normal-phase HPLC.

Esterification assay of all-trans-retinol and all-trans-13,14-dihydroretinol. HEKK-LRAT cells were homogenized in 250 mM sucrose, 10 mM Tris-HCl with the aid of a douncer. RPE microsomes were prepared as previously described. Stecher, et al. J Biol Chem 274:8577-8585, 1999. A substrate solution, 2 μL of 1 mM stock in DMF, was added to a 1.5 ml Eppendorf tube containing 20 μL of 10% BSA and 20 μL of UV-treated RPE microsomes or 100 μL of membrane homogenate of HEKK-LRAT cells and 10 mM BTP (pH 7.5) buffer to a total volume of 200 μL. The reactions were incubated at 37° C. for 10 min. Retinoids were extracted with 300 μL of methanol and 300 μL of hexane. Then 100 μL of the hexane extract was analyzed by normal-phase HPLC, using first 0.5% ethyl acetate in hexane for 10 min to separate retinyl esters and then 20% ethyl acetate in hexane for an additional 10 min to separate retinols. Elution was monitored at 290 nm and 325 nm.

### EXAMPLE 4

Cloning of the cDNA of Mouse and Monkey CRTISO-Like Proteins

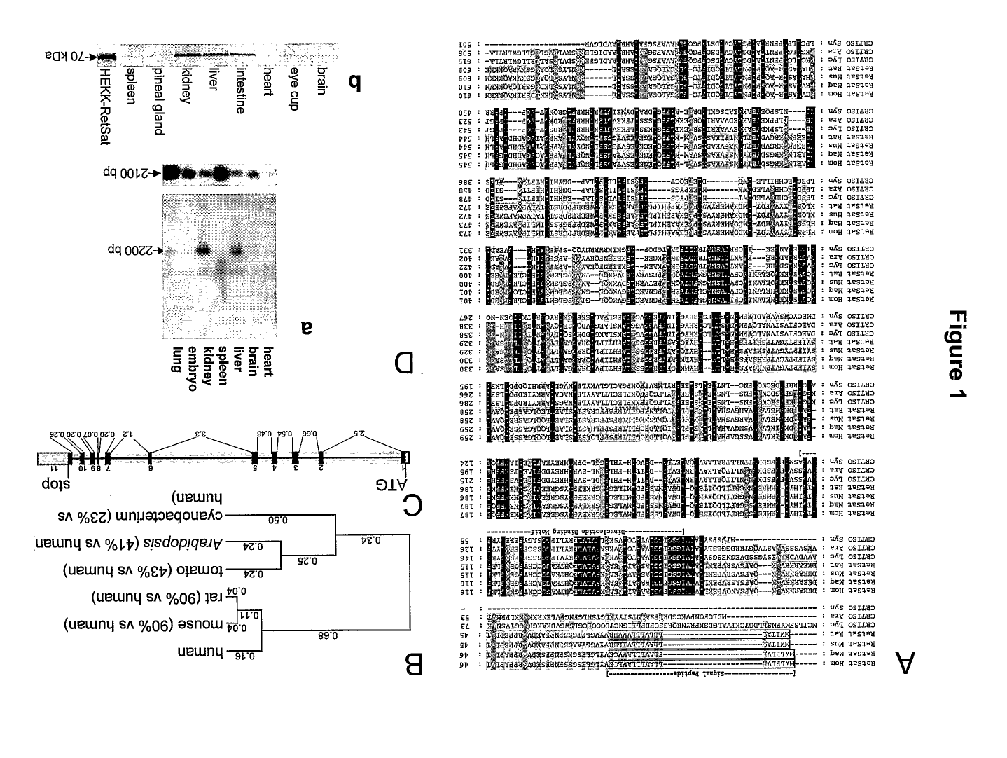

The protein sequences of tomato and A. thaliana CRTISO were used to search for similar proteins in other species. We found proteins that share extensive similarity over the entire length of the protein in several phyla, from bacterial, archaebacterial, and fungal phytoene dehydrogenases to other phytoene dehydrogenase and CRTISO-like proteins in other plants and higher eukaryotes. A family of highly conserved proteins was found in many chordate species but not in non-chordates. This chordate CRTISO-like protein family has members in vertebrates such as man, mouse, rat, chicken, and zebrafish and pufferfish (Fugu rubipres and Tetraodon nigroviridis), as well as invertebrates such as the ascidians Ciona intestinalis and Ciona savignyi. The CRTISO-like ascidian proteins share many conserved residues with the related vertebrate proteins as judged by the translation of the available ascidian genomic sequence (63% conserved substitutions including 41% identical residues compared with human). The alignment of the human, monkey, mouse, and rat protein sequences to CRTISO from tomato, A. thaliana and cyanobacterium Synechocystis sp. (strain PCC 6803) is represented in FIG. 1A. Vertebrate CRTISO-like proteins are named RetSat after the catalytic activity observed for this enzyme (see the following sections). A phylogenetic dendogram based on a neighbor-joining algorithm appears to be monophyletic (FIG. 1B) and indicates that the proteins found in vertebrates are related to plant CRTISO (41-43% conserved substitutions including 25-27% identical residues). Thus the ancestral member of plant CRTISO and vertebrate RetSat appeared before the divergence of plant and animal kingdoms. Not only do mouse, human, and rat RetSat proteins share extensive homology throughout their sequence, but the genes coding for these proteins have the same exon-intron arrangement, with the intron breaks at the same place in the aligned protein sequence. The human gene encompasses 12 kbp of genomic DNA and 11 exons on the minus strand of chromosome 2 (FIG. 1C). The 3 kbp cDNA of the human RetSat protein (accession number gi31377747) encodes a protein of 65 kDa, based on theoretical mass calculations of the translated sequence. There is an in-frame stop codon 54 bp upstream of the potential translation initiation site without intervening splice acceptors, which indicates that the 5′-end of the cDNA matches the amino terminus of the protein.

A putative dinucleotide-binding domain, also observed in a protein superfamily that includes FAD-binding mammalian monoamine oxidases and protoporphyrinogen oxidases as well as phytoene desaturases, is located at the N-terminal portion of RetSat. Wierenga, et al. J Mol Biol 187:101-107, 1986; Dailey, et al. J Biol Chem 273:13658-13662, 1998. Another apparent feature is the canonical signal sequence that targets the nascent protein to the membrane of the endoplasmic reticulum (ER). Blobel, et al. Symp Soc Exp Biol 33:9-36, 1979. The hydrophobic stretch from residue 568 to 588 is the most likely transmembrane domain.

The cDNA for mouse and macaque monkey RetSat orthologous proteins was cloned from reverse transcribed RNA from the retina and RPE. Sequencing of several independent clones ensured that the sequence was verified. The sequence of the submitted mouse RetSat cDNA (AY704159) has five bases that are different from the sequence available in the database (gi18483252), two of which result in amino acid changes. In a previous study, rat RetSat expression and other gene products were identified as downregulated in rat mammary adenocarcinomas, and the rat cDNA was tentatively designated rat mammary tumor-7 (RMT-7). Wang, et al. Oncogene 20:7710-7721, 2001. No further biochemical characterization of the enzyme was carried out. The sequence we deposited to GenBank for mouse RetSat (AY704159) corresponds perfectly to multiple EST sequences and it is more similar to human RetSat than the sequence currently available in the database. Monkey RetSat protein (GenBank submission AY707524) has 97% conserved substitutions, including 94% identical residues with the human protein sequence available in the database (gi46329587).

### EXAMPLE 5

### Characterization of the Tissue Distribution and Subcellular Localization of Mouse RetSat Protein

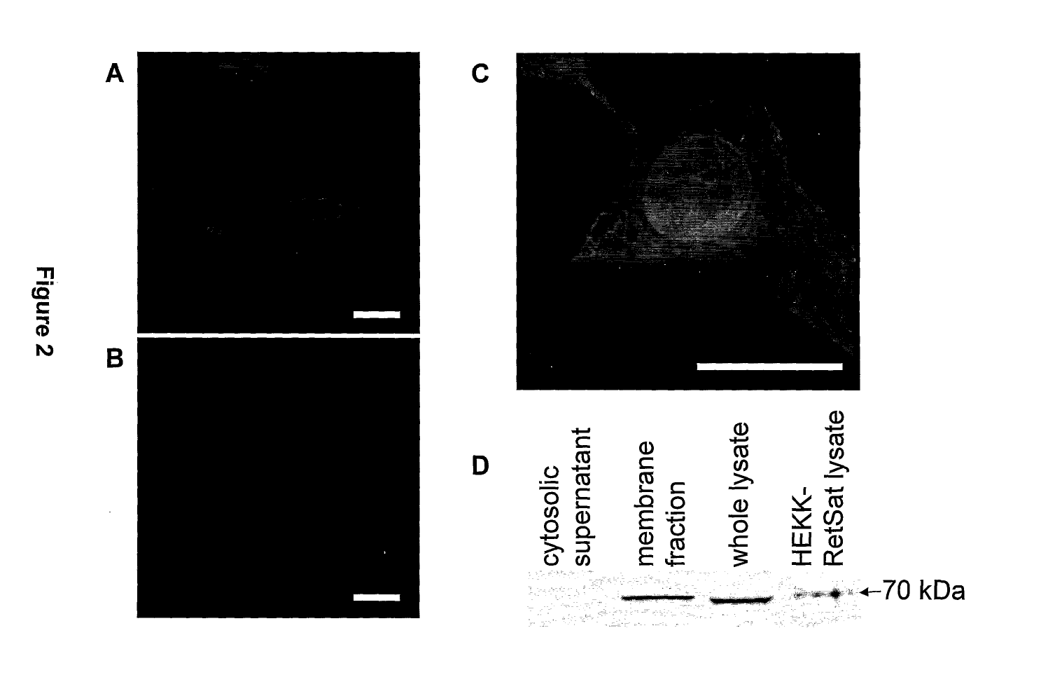

Mouse RetSat expression was examined by Northern blot analysis using a radiolabeled antisense RetSat probe and a comercially available premade blot containing equal amounts of RNA from various tissues. RetSat mRNA appears as a 2200 bp transcript expressed predominantly in the liver and kidney among the tissues examined (FIG. 1D a, top panel). We also examined the expression of the 2100 bp non-muscle β-actin mRNA in the same tissues to verify the quality of the RNA on the blot (FIG. 1D a, bottom panel). Alonso, et al. J Mol Evol 23:11-22, 1986. Greater amounts of RNA from spleen and lung tissues are present, based on the level of actin detected. In spite of this, RetSat mRNA cannot be detected in the corresponding lanes of spleen and lung in the top panel of FIG. 1D (a), while it is clearly present in the kidney and liver at the same exposure of the blot (30 min). Very low levels of Retsat were detectable only after much longer exposure of the blot (5 h) in other tissues beside kidney and liver. This was confirmed by RT-PCR indicating that RetSat is expressed predominantly in the kidney and liver and a very low levels in many other tissues examined. A rabbit polyclonal antiserum and a monoclonal antibody were prepared against recombinant mouse RetSat. For both mice and rabbit immunogens a bacterially expressed fragment of the mouse RetSat protein was used as antigen. The recombinant protein fragment was chosen to eliminate the putative dinucleotide-binding domain that may result in cross-reaction with related proteins. Glycosylation-deficient HEK cells obtained from Dr. Khorana, HEKK, were transfected with the tetR gene and mouse RetSat cDNA under the control of the tetracycline (Tet)-inducible promoter. Reeves, et al. Proc Natl Acad Sci USA 99:13419-13424, 2002. Stable clones of transfected cells were selected, pooled, and used for further analysis. These cells were designated HEKK-RetSat. Both polyclonal (FIG. 1D b) and monoclonal antibodies (FIG. 2D) reacted with a specific protein of 70 kDa, similar to the predicted mass of mouse RetSat protein and identical to the mass of the protein detected in Tet-induced HEKK-Retsat cells. Equal amounts of protein from several tissues were analyzed by SDS-PAGE and immunoblotting with anti-RetSat polyclonal antibody. RetSat protein was detected in many tissues, with the highest expression in liver, kidney, and intestine (FIG. 1D b). This expression pattern was also confirmed by immunoblotting with the monoclonal anti-RetSat antibody.

FIG. 1 shows the identification of vertebrate proteins with similarity to plant and cyanobacteria CRTISO. (A) Sequence comparison of human RetSat (RetSat Hom-gi46329587), macaque-monkey RetSat (RetSat Maq-AY707524 submitted sequence) mouse RetSat (RetSat Mus-AY704159 submitted sequence), and rat RetSat (RetSat Rat-gi34855900) with tomato CRTISO (CRTISO Lyc-gi19550437), Arabidopsis CRTISO (CRTISO Ara-gi42561764), and cyanobacterial CRTISO (CRTISO Syn-gi16331999). White letters on a black background represent identical residues. White letters on gray background represent conserved substitutions in all but one of the species examined, while black letters on light gray background indicate substitutions conserved in four of the seven species examined. Dashed lines represent gaps introduced to maximize the alignment. The alignment was built using the program T-Coffee and the matrix BLOSUM62 with gap penalties: existence-11, extension-1. Sequence-based predictions such as the signal peptide and a putative dinucleotide binding motif are indicated. Henikoff, et al. Proc Natl Acad Sci USA 89:10915-10919, 1992. A phylogenetic tree of CRTISO-like enzymes was built using the Clusta1W-neighbor-joining distance algorithm with numbers indicating evolutionary distances (B). Saitou, et al. Mol Biol Evol 4:406-425, 1987. The percent similarity to human RetSat is indicated in parentheses beside gene name. (C) Gene structure of human RetSat as it is found on minus strand of chromosome 2 from 85,556,195 to 85,543,754. Numbered black boxes indicate exons, white boxes indicate untranslated regions, and lines represent introns. The length of each intron is indicated in kbp. The start (ATG) and stop of translation are also indicated. (D) Tissue distribution of mouse RetSat. (a) Northern Blot analysis of mouse RetSat expression in various mouse tissues (top panel) indicates that mouse RetSat is expressed predominatly in the liver and kidney among the tissues examined. Control hybridization was performed by stripping and reprobing of the same blot using an antisense probe to non-muscle β-actin (bottom panel). The size of detected transcripts is shown at the right side of the panels. Lysates of various mouse tissues containing 10 μg of protein per lane were subjected to immunoblotting using rabbit polyclonal anti-mouse RetSat serum (b). The lane labeled HEKK-RetSat shows the immunoreactivity of the mouse RetSat protein from the lysate of Tet-induced, HEKK-RetSat cells corresponding to 1 μg of total loaded protein. There is no immunoreactive band in the lysate of untransfected cells immunoblotted with either rabbit polyclonal or mouse monoclonal antibody. The apparent molecular mass of mouse RetSat is 70 kDa and is indicated on the right side of the panel.

The subcellular localization of mouse RetSat protein was studied by immunocytochemistry using the anti-RetSat monoclonal antibody. First, the antibody was tested for its specificity by staining Tet-induced HEKK-RetSat cells and untransfected cells, which showed no reaction with the antibody (FIGS. 2A and B). The staining of HEKK-RetSat cells matches that of the perinuclear and ER membrane, indicating that mouse RetSat is targeted to the ER compartment in transfected cells (FIG. 2C). There is no cytoplasmic or plasma membrane staining. Subcellular fractionation confirmed that RetSat was a membrane-associated protein not detectable by immunoblotting of the cytosolic supernatant of mouse liver cells with monoclonal antibody (FIG. 2D). A protein that migrates with an apparent molecular weight of 70 kDa was seen in both liver microsomal membranes and HEKK-RetSat lysate (FIG. 2D). This protein was absent in the lysate of untransfected cells using either RetSat monoclonal antibody or polyclonal anti-RetSat antiserum.

FIG. 2 shows the subcellular localization of mouse RetSat in transfected cells. The anti mouse-RetSat monoclonal antibody was used to stain Tet-induced HEKK-RetSat transfected cells (A) and untransfected cells (B). HEKK-RetSat cells stained with the anti-RetSat monoclonal antibody examined under higher magnification show the perinuclear and reticular membrane localization of RetSat in transfected cells (C). The scale bar represents 20 μm. (D) Subcellular analysis of RetSat protein in mouse liver cells. Immunoblotting of equal amounts of protein from the cytosolic supernatant, postnuclear membrane fraction, and whole cell lysate of mouse liver cells indicates that the RetSat protein is membrane associated. An immunoreactive band of a protein with apparent molecular mass of 70 kDa was identified as the mouse RetSat protein, confirmed by its presence in the lysate of Tet-induced HEKK-RetSat cells. The blots were probed with the anti-mouse RetSat monoclonal antibody.

### EXAMPLE 6

### Tomato CRTISO and Mouse RetSat Exhibit Different Enzyme Activities

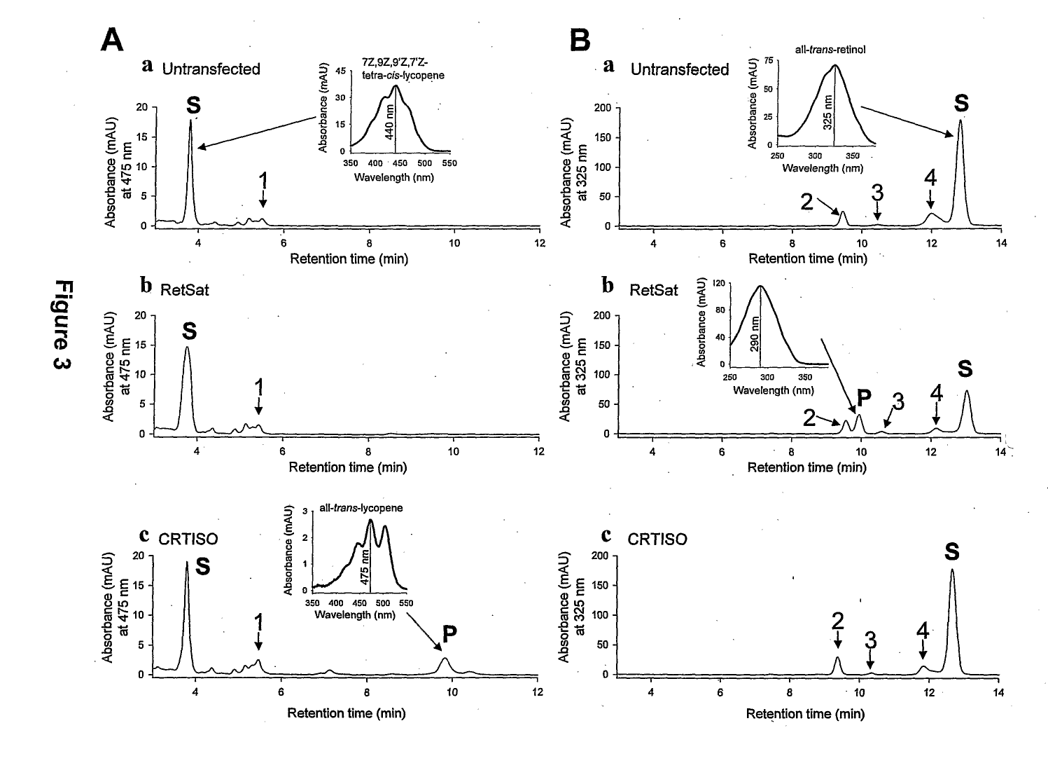

Tomato CRTISO was cloned from RNA isolated from the skin and pulp of a fresh red tomato fruit. Tomato CRTISO was expressed in HEKK cells under the control of an inducible promoter. The natural substrate of tomato CRTISO, 7Z,9Z,9′Z,7′Z-tetra-cis-lycopene, was isolated by organic extraction of a tangerine tomato, which accumulates 7Z,9Z,9′Z,7′Z-tetra-cis-lycopene. Zechmeister, et al. Proc Natl Acad Sci USA 21:468-474, 1941. The tangerine tomato extract consisted mostly of 7Z,9Z,9′Z,7′Z-tetra-cis-lycopene (greater than 90%) as determined by reverse-phase HPLC analysis and the UV absorbance spectrum of the main peak, which matched published spectra (peak S, FIG. 3A a). The 7Z,9Z,9′Z,7′Z-tetra-cis-lycopene exhibits a shifted absorbance maxima λₘₐₓ of 440 nm compared with all-trans-lycopene λmax of 475 nm and has a distinct UV absorbance spectrum. Hengartner, et al. Helvetica Chimica Acta 75:1848-1865, 1992. Untransfected HEKK, RetSat- and CRTISO-expressing cells were incubated in the presence of 7Z,9Z,9′Z,7′Z-tetra-cis-lycopene in the dark (FIG. 3A). The products of the reaction were analyzed by reverse-phase HPLC System I. There was no difference in the profile of eluted carotenoids from either untransfected cells or RetSat-expressing cells (FIGS. 3A a and b). As expected, CRTISO-expressing cells converted the substrate 7Z,9Z,9′Z,7′Z-tetra-cis-lycopene (S in FIG. 3A c) into all-trans-lycopene, with a λmax of 475 nm (P in FIG. 3A c). All-trans-lycopene was identified based on its absorbance spectrum and co-elution with an available standard obtained from Dr. Kurt Bernhard (CaroteNature GmbH, Lupsingen, Switzerland) and Dr. Regina Goralczyk (Roche Vitamins Ltd., Basel, Switzerland). CRTISO also catalyzed the conversion of 7Z,9Z,9′Z,7′Z-tetra-cis-lycopene into a compound labeled 1 in FIG. 3A, which we tentatively identified as 7,9 di-cis-lycopene isomer based on its absorbance spectrum. Hengartner, et al. Helvetica Chimica Acta 75:1848-1865, 1992 This observation would suggest a two-step reaction mechanism for CRTISO, in which both cis- bonds of first one end, then the other of the carotenoid are isomerized. Thermal or light-induced isomerization can convert 7Z,9Z,9′Z,7′Z-tetra-cis-lycopene into the 7Z,9Z-di-cis isomer at a slower rate, as this compound was also present in the original tomato extract (peak 1, FIGS. 3A a and b) and reported by other investigators. Bartley, et al. Eur J Biochem 259:396-403, 1999.

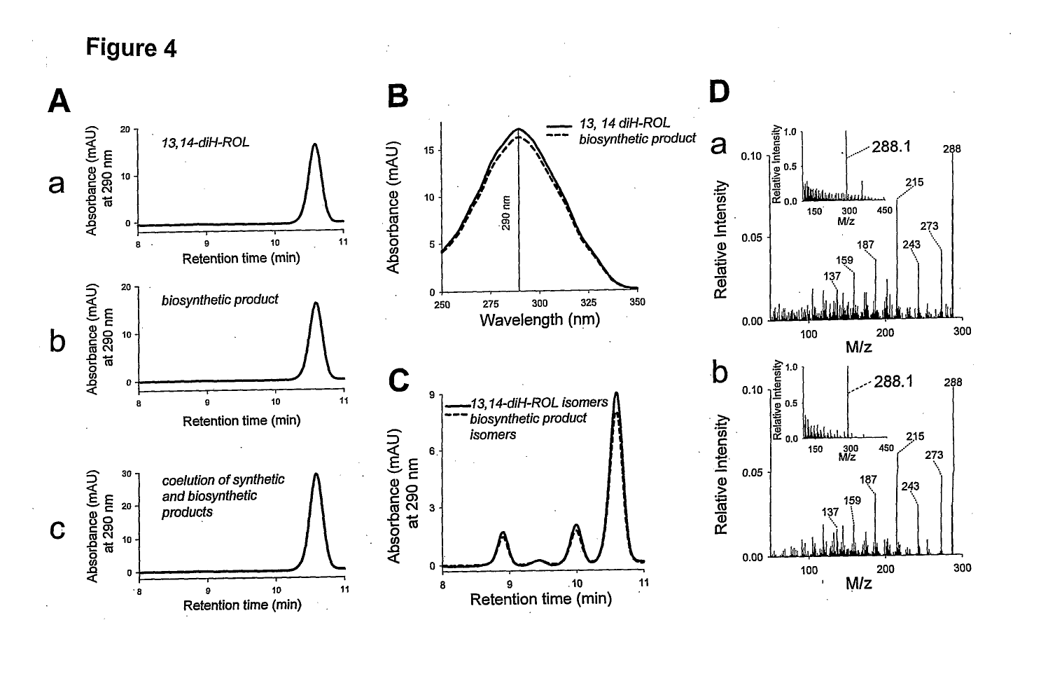

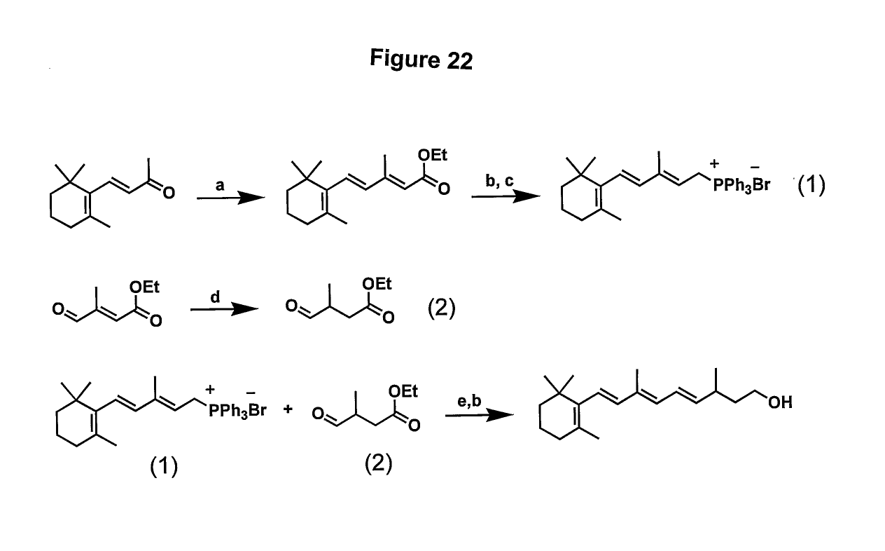

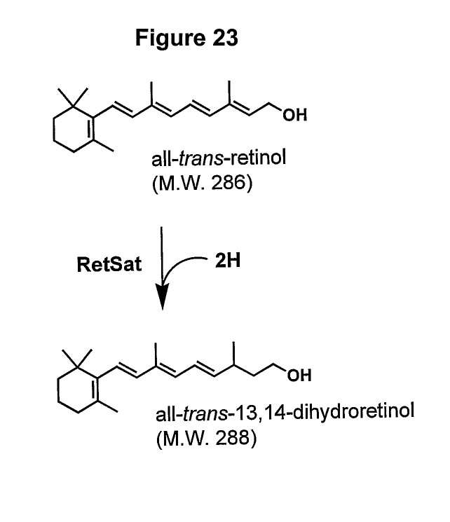

Our analysis of various carotenoid and retinoid substrates led us to investigate the activity of RetSat in the presence of all-trans-retinol. When the products of the reaction were examined by normal-phase HPLC, we noticed that RetSat-expressing cells incubated with all-trans-retinol (peak S, FIG. 3B) in the dark converted it to a less polar compound whose λmax was 290 nm (peak P, FIG. 3B b). The peak was absent in untransfected and CRTISO-expressing cells. Cis-isomers of retinol (peaks 2, 3, and 4, FIG. 3B) generated during the overnight incubation were present in all cells regardless of background. Based on the hypsochromic-shifted UV absorbance maximum of 290 nm, we deduced that the new compound has one less double bond compared with the parent compound retinol exhibiting a λₘₐₓ of 325 nm. A survey of the literature indicates that all-trans-13,14-dihydroretinal exhibits a maximum absorption at 289 nm. Yan, et al. J Biol Chem 270:29668-29670, 1995. To prove the hypothesis that the unknown compound is all-trans-13,14-dihydroretinol, it was chemically synthesized as depicted in FIG. 22, purified by BPLC and characterized by ¹H-NMR spectrum. FIG. 22 shows synthesis of all-trans-13,14-dihydroretinol. a) (EtO)₂P(O)CH₂COOEt. NaH, THF, rt, 24 hr; b) LiAlH₄, Et₂O, O° C., 30 min; c) Ph₃P.HBr, MeOH, rt, 24 hr; d) H₂ (bar), MeOH, Pd/C, rt, 24 hr; e) tert-BuOK, 18-crown-6, CH₂Cl₂, rt to −78° C. to rt, 12 hr. The unknown compound produced by RetSat-expressing cells was purified by collecting the appropriate fraction from a normal-phase HPLC. The purity of the unknown biosynthetic compound and synthetic all-trans-13,14-dihydroretinol was verified by normal-phase HPLC (FIGS. 4A a and b). The amount of the purified unknown compound precluded us from conducting its 1H-NMR analysis. However, both all-trans-13,14-dihydroretinol and the unknown compound exhibit the same chromatographic properties on normal-phase HPLC, as they co-eluted as one peak when combined (FIG. 4A c). The two compounds have identical UV absorbance spectra (FIG. 4B), and light-induced isomerization of equal amounts of the two compounds generates a series of cis-isomers identical in both elution profile and intensity (FIG. 4C). Acetylation of the synthetic all-trans-13,14-dihydroretinol and the extracted compounds produced ester compounds that co-eluted on a normal-phase HPLC (data not shown). More importantly, MS-analysis revealed that the biosynthetic compound has an m/z mass of 288, an increase of 2 Daltons from the mass of the parent compound, all-trans-retinol (m/z=286) (FIG. 4D a, inset). This observed mass is the same as the mass of synthetic all-trans-13,14-dihydroretinol (FIG. 4D b, inset). The MS fragmentation pattern of the both synthetic and biosynthetic compounds is identical (FIGS. 4D a and b). Since C13 becomes a chiral center in 13,14-dihydroretinol, further NMR analysis will be necessary to establish the absolute configuration of the biosynthetic compound. These findings lead us to propose that RetSat catalyzed the saturation reaction of the 13-14 double bond of all-trans-retinol as depicted in FIG. 23. FIG. 23 shows the eaction catalyzed by RetSat converting all-trans-retinol into all-trans-13,14-dihydroretinol.

FIG. 3 shows the enzyme activities of tomato CRTISO and mouse RetSat in transfected cells. (A) Analysis of the effect of tomato CRTISO and mouse RetSat on the conversion of 7Z,9Z,9′Z,7′Z-tetra-cis-lycopene into all-trans-lycopene. Cells were incubated with 7Z,9Z,9′Z,7′Z-tetra-cis-lycopene substrate (S) extracted and examined by reverse-phase HPLC System I for the conversion of S into all-trans-lycopene product (P). The analysis indicates that the conversion occurs in cells expressing tomato CRTISO (c) but not in untransfected (a) or RetSat-expressing cells (b). A compound whose absorbance spectrum corresponds to 7,9 di-cis-lycopene was observed in all cells and more intensely in CRTISO-expressing cells (indicated by Ruiz, et al. J Biol Chem 274:3834-3841, 1999). (B) Analysis of the effect of tomato CRTISO and mouse RetSat on the conversion of all-trans-retinol into a new product. Cells were incubated with all-trans-retinol substrate (S) extracted and examined by normal-phase HPLC for the conversion of S into a novel product (P) whose maximum absorbance peak is 290 nm. The analysis indicates that the conversion occurs in cells expressing mouse RetSat (b) but not in untransfected (a) or CRTISO-expressing cells (c). Additional peaks with absorbance spectra corresponding to 13-cis-retinol, 9,13-di-cis-retinol, and 9-cis-retinol were observed in all cells regardless of background and are most likely the result of thermal isomerization. Batten, et al. J Biol Chem 279:10422-10432, 2004; Imanishi, et al. J Cell Biol 164:373-383, 2004; Kuksa, et al. Vision Res 43:2959-2981, 2003. The experiment was performed in duplicate samples and repeated.

FIG. 4 shows the identification of the biosynthetic product of the conversion of all-trans-retinol by mouse RetSat. The HPLC-purified biosynthetic product of the RetSat reaction was compared to 13,14-dihydroretinol for its elution characteristics on normal-phase HPLC (A). The retention times for both all-trans-13,14-dihydroretinol (a) and the biosynthetic product (b) are identical and when mixed the two compounds co-elute as a single peak (c). The absorbance spectrum for the two compounds is identical with a maximum absorbance at 290 nm (B). Both all-trans-13,14-dihydroretinol and the biosynthetic compound generate the same pattern of isomers following light-induced isomerization (C). Electron-impact MS analysis of the biosynthetic product (a) and all-trans-13,14-dihydroretinol (b) shows they have the same mass of 288 m/z, corresponding to retinol plus 2H (D). The base peak is shown in the inset. The MS fragmentation patterns of biosynthetic compound (a) and all-trans-13,14-dihydroretinol compound (b) show that they generate ions of the same mass and relative intensity.

### EXAMPLE 7

### Substrate Selectivity of Mouse RetSat

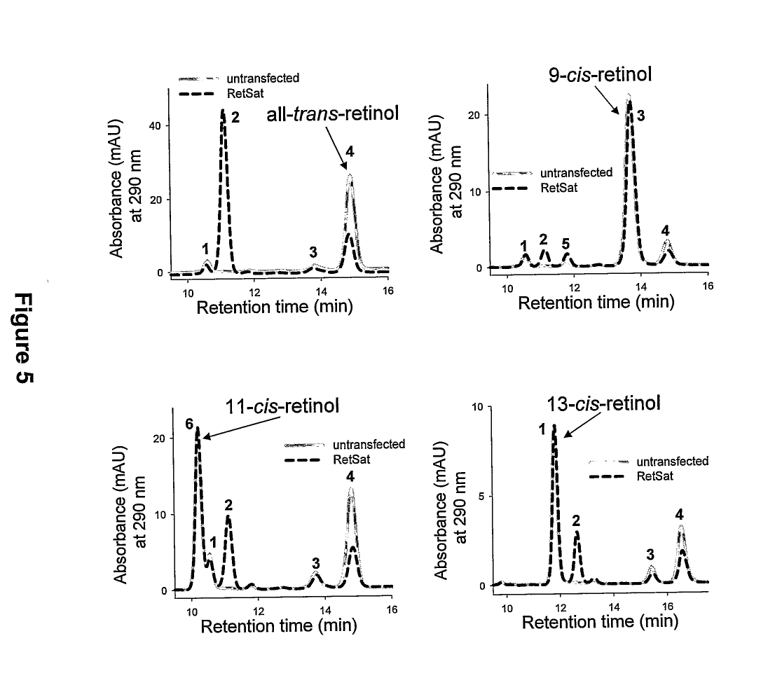

The substrate specificity of RetSat was investigated using purified isomers of retinol. RetSat-expressing cells were incubated overnight with the different retinol isomers. The isomers were more than 95% pure at the time of addition as confirmed by normal-phase HPLC analysis. However, during the overnight incubation cis-isomers of retinol converted to all-trans isomer and then back to other cis-retinol isomers, complicating the interpretation of results. All-trans-retinol was clearly a good substrate for RetSat based on the amount of all-trans-13,14-dihydroretinol produced (peak 2, FIG. 5 top left) and the amount of all-trans-retinol utilized (peak 4, FIG. 5, solid-gray and short dash-black trace representing untransfected and RetSat-expressing cells, respectively). Only all-trans-13,14-dihydroretinol product was formed in all reactions and no cis isomers were detected. In all assays with cis-retinol isomers the amount of all-trans-13,14-dihydroretinol produced correlates with the amount of all-trans-retinol present and utilized in the reaction. Meanwhile, the amount of cis-retinol substrate stayed the same in either RetSat-expressing (short dash-black trace) or untransfected cells (solid-gray trace, FIG. 5). Based on this evidence, it appears that all-trans-retinol was the preferred substrate for RetSat. The all-trans-13,14-dihydroretinol found in cells incubated with cis-retinol isomers was produced from all-trans-retinol derived by spontaneous isomerization of the cis-retinol substrate.

FIG. 5 shows the isomeric form of the substrate of mouse RetSat. Tet-induced HEKK-RetSat cells were incubated overnight with pure isomers of retinol (>95% pure by HPLC, assayed before incubation). Following incubation retinoids were extracted and analyzed by normal-phase HPLC. The appearance of 13,14-dihydroretinol isomers was monitored at 290 nm since the absorbance maxima of most isomers of 13,14-dihydroretinol differ by less than 5 nm from 290 nm, the λmax of all-trans-13,14-dihydroretinol (spectra not shown). In each panel an arrow indicates the substrate investigated to distinguish it from the additional retinol isomers that were generated by thermal isomerization during overnight incubation in tissue culture. Numbers indicate the identity of eluted peaks based on absorbance spectra and comparison with pure standards, specifically, 13-cis-retinol, all-trans-13,14-dihydroretinol, 9-cis-retinol, all-trans-retinol, 9,13-di-cis-retinol, and 11-cis-retinol. Ruiz, et al. J Biol Chem 274:3834-3841, 1999; Batten, et al. J Biol Chem 279:10422-10432, 2004; Imanishi, et al. J Cell Biol 164:373-383, 2004; Kuksa, et al. Vision Res 43:2959-2981, 2003; Chou, et al. J Biol Chem 277:25209-25216, 2002; Duester, et al. Chem Biol Interact 143-144, 201-210, 2003. No isomers of 13,14-dihydroretinol were detected other than the all-trans isomer. The retention times in the bottom right panel are slightly longer due to variations in solvent system. The experiment was performed in triplicate and repeated.

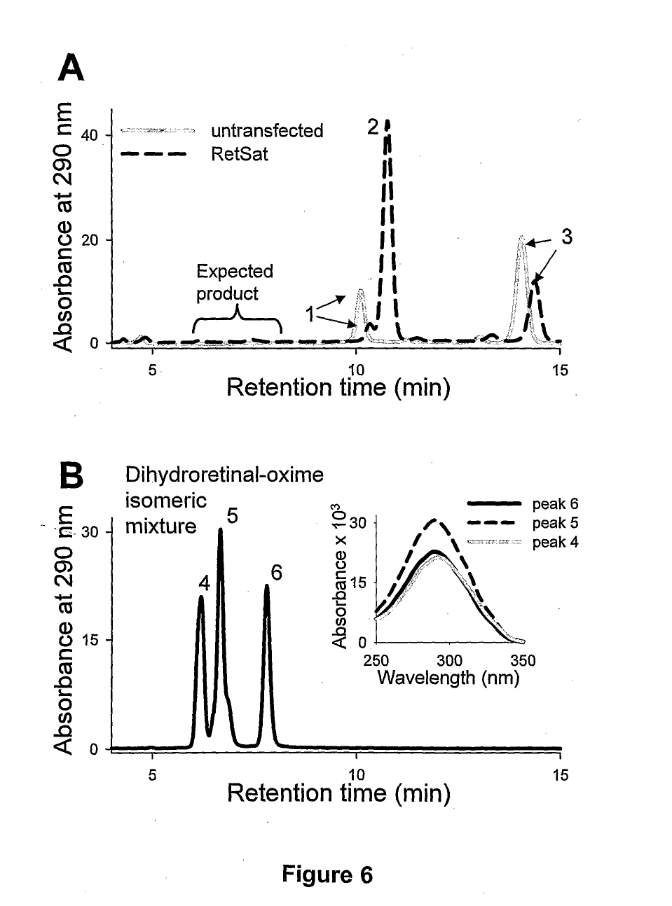

We also examined whether retinal or retinoic acid could be saturated by RetSat to corresponding 13,14-dihydroretinal or 13,14-dihydroretinoic acid. Retinal was almost completely reduced to retinol by incubation with cells as evident by the barely detectable levels of retinal-oximes and the appearance of retinol (peak 3 FIG. 6A). In Retsat-expressing cells all-trans-retinol was then readily converted to all-trans-13,14-dihydroretinol (peak 2, FIG. 6A). Synthetic 13,14-dihydroretinal-oxime derivatives (λₘₐₓ=290 nm) were examined on the same HPLC system to establish product elution conditions (FIG. 6B and inset spectra). However, no dihydroretinal-oximes were detected in RetSat-expressing cells incubated with retinal (6-8 min elution time FIG. 6A). It was not possible to conclusively establish whether retinal is a substrate for RetSat given the rapid conversion of retinal to retinol in cultured cells.

FIG. 6 shows RetSat activity towards all-trans-retinal. (A) Analysis of retinal conversion in RetSat-expressing cells. Tet-induced HEKK-RetSat or untransfected cells were incubated overnight with pure all-trans-retinal (>99% pure by HPLC, assayed before incubation). Following incubation retinals were derivatized with hydroxylamine, extracted and analyzed by normal-phase HPLC. The appearance of syn and anti-oximes of 13,14-dihydroretinal was monitored at 290 nm (expected 6-8 minutes after injection, as indicated). Peak numbers represent 13-cis-retinol, all-trans-13,14-dihydroretinol and all-trans-retinol. Ruiz, et al. J Biol Chem 274:3834-3841, 1999; Batten, et al. J Biol Chem 279:10422-10432, 2004; Kuksa, et al. Vision Res 43:2959-2981, 2003. (B) Synthetic standards of 13,14-dihydroretinal derivatized with hydroxylamine were examined by normal-phase HPLC in order to establish product elution profile. Inset shows the spectra of the different isomers of 13,14-dihydroretinal-oximes.

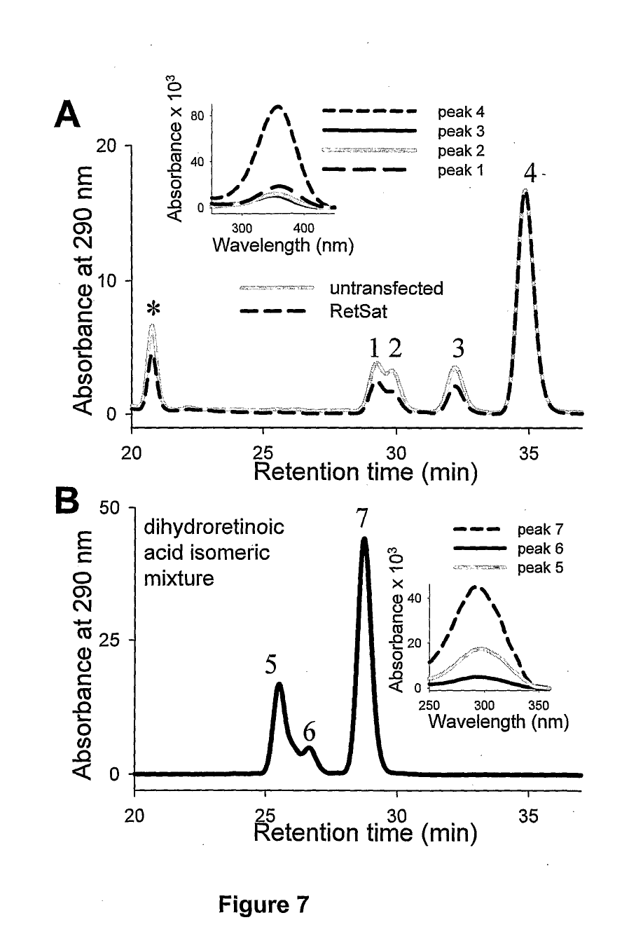

Incubation of cells with retinoic acid indicated that it is not substrate for saturation by RetSat (FIG. 7A). Synthetic 13-14-dihydroretinoic acid standards were examined on the same HPLC system to establish their elution conditions (FIG. 7B). Even though, 13-cis retinoic acid (peak 1, FIG. 7A) coelutes with all-trans-13,14-dihydroretinoic (peak 7, FIG. 7B) the absorbance spectrum of the two compounds is different (FIGS. 7A and B insets) and allowed us to conclude that 13,14-dihydroretinoic acid cannot be detected in RetSat-expressing cells incubated with retinoic acid.

FIG. 7 shows RetSat activity towards all-trans-retinoic acid. (A) Analysis of retinoic acid conversion in RetSat-expressing cells. Tet-induced HEKK-RetSat or untransfected cells were incubated overnight with pure all-trans-retinoic acid (>90% pure by HPLC, assayed before incubation). Following incubation retinoic acid was extracted and analyzed by reverse-phase HPLC System II. The appearance of 13,14-dihydroretinoic acid isomers was monitored at 290 nm (expected 25-30 minutes after injection). Peak numbers represent 13-cis-retinoic acid, 9,13-di-cis-retinoic acid, 9-cis-retinoic acid and all-trans-retinoic acid. Ruiz, et al. J Biol Chem 274:3834-3841, 1999; Batten, et al. J Biol Chem 279:10422-10432, 2004; Kuksa, et al. Vision Res 43:2959-2981, 2003; Imanishi, et al. J Cell Biol 164:373-383, 2004. (B) Mixture of isomers of synthetic standards of 13,14-dihydroretinoic acid were examined by reverse-phase HPLC System II in order to establish product elution profile. Inset shows the spectra of the different isomers of 13,14-dihydroretinoic acid. Star (*) indicates an unrelated compound. The experiment was performed in triplicate samples and repeated.

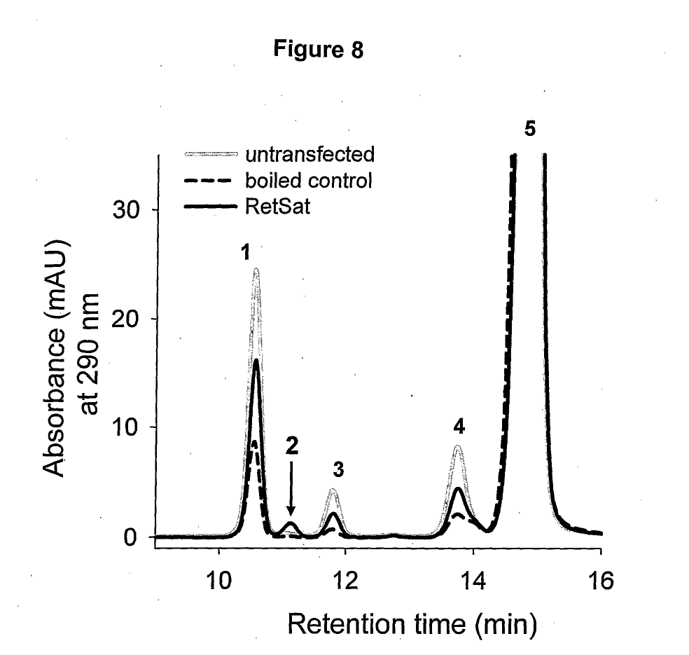

To avoid thermal isomerization, the substrates were examined in homogenized microsomal RetSat membranes with or without additional cofactors. Membrane homogenate from RetSat-expressing cells was incubated with all-trans-retinol, and the product of the reaction was examined by normal-phase HPLC. There was little all-trans-13,14-dihydroretinol produced, as indicated by the elution peak labeled 2, solid-black trace, FIG. 8. The addition of reduced dinucleotide cofactors NADH or NADPH had no effect on the yield of the reaction. For control, membranes from untransfected cells (gray trace) and boiled membranes from RetSat-expressing cells (short dash-black trace) showed no activity. Cell homogenization destroyed the activity of RetSat most likely by affecting the redox status or by the loss of a key cofactor. The low activity of RetSat in vitro is not surprising given the well-documented labile nature of CRTISO and phytoene desaturase enzymes. Park, et al. Plant Cell 14:321-332, 2002; Cunningham, et al. Annual Review of Plant Physiology and Plant Molecular Biology 49:557-583, 1998.

FIG. 8 shows RetSat activity in homogenized cells. Untransfected cells (solid-gray trace) or Tet-induced HEKK-RetSat cells (solid-black trace) were homogenized and incubated with all-trans-retinol substrate, followed by retinoid extraction and normal-phase HPLC analysis. The elution profile was monitored at 290 nm for the appearance of all-trans-13,14-dihydroretinol. In control samples (short dash-black trace) cell homogenate from HEKK-RetSat cells was boiled 10 min at 95° C. prior to incubation with substrate. The addition of 0.4 mM NADH or NADPH had no effect on the yield of all-trans-13,14-dihydroretinol. The experiment was performed in duplicate.

### EXAMPLE 8

All-trans-13,14-dihydroretinol can be Detected in Several Tissues of Animals Maintained on a Normal Diet.

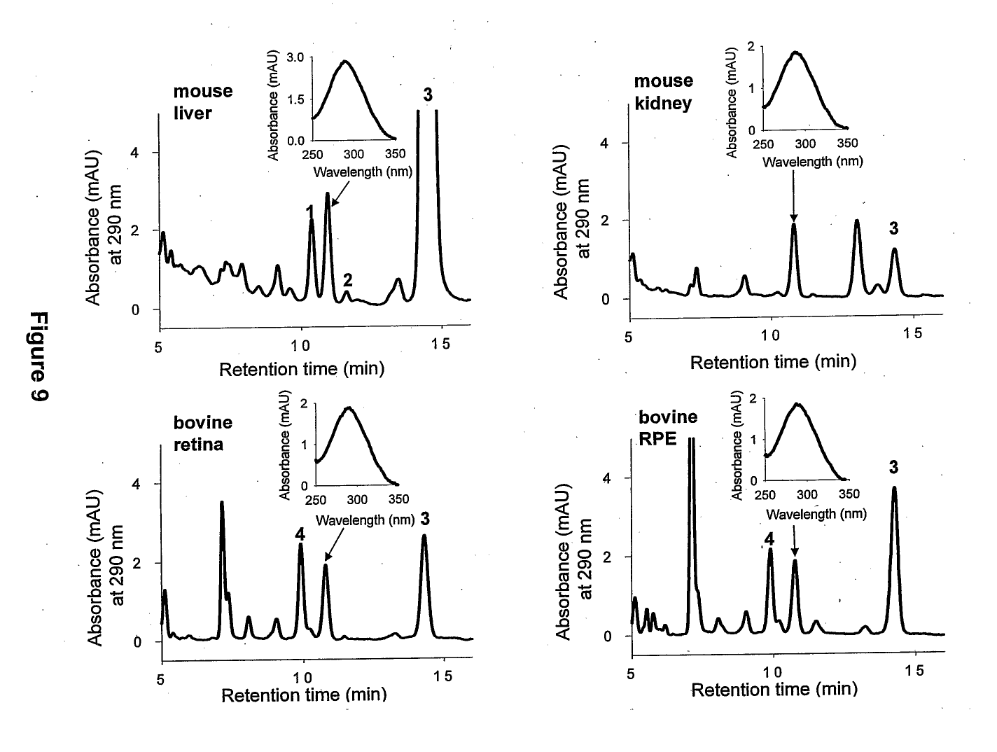

The presence of RetSat in major organs such as the liver and kidney led us to investigate if all-trans-13,14-dihydroretinol could be detected in tissues. All-trans-13,14-dihydroretinol could be readily detected by normal-phase HPLC analysis of mouse liver and kidney and bovine retina and RPE (FIG. 9). All-trans-13,14-dihydroretinol was recognized based on its UV absorbance spectrum and chromatographic retention time, which both matched those of the synthetic compound. Retinol isomers such as 13-cis-retinol (peak 1), 9,13-di-cis-retinol (peak 2), all-trans-retinol (peak 3) and 11-cis-retinol (peak 4) (FIG. 9) were also detected and recognized based on available standards and UV absorbance maxima. We conclude that all-trans-13,14-dihydroretinol represents a minor but readily detectable retinoid in many tissues examined from animals maintained on a normal diet not supplemented with vitamin A.

FIG. 9 shows the identification of all-trans-13,14-dihydroretinol in various tissues. Retinoids were extracted from mouse liver (0.3 g, top left panel), kidney (0.2 g, top right panel), bovine retina (0.2 g, bottom left panel), and RPE (0.2 g, bottom right panel) and examined by normal-phase HPLC. The elution of 13,14-dihydroretinol was monitored at 290 nm. Based on its retention time and absorbance spectrum a peak corresponding to all-trans-13,14-dihydroretinol was identified in all tissues examined; it elutes on normal-phase HPLC between 13-cis-retinol (1) and 9,13-di-cis-retinol (2). Other peaks corresponding to all-trans-retinol (3) and 11-cis-retinol (4, bovine retina and RPE) were also identified. The experiment was performed in duplicate from tissues of different animals. The yield of all-trans-13,14-dihydroretinol was slightly higher (<10%) by saponification of the extract before HPLC analysis.

### EXAMPLE 9

Esterification of all-trans-13,14-dihydroretinol in RPE Microsomes and HEKK-LRAT Cells.

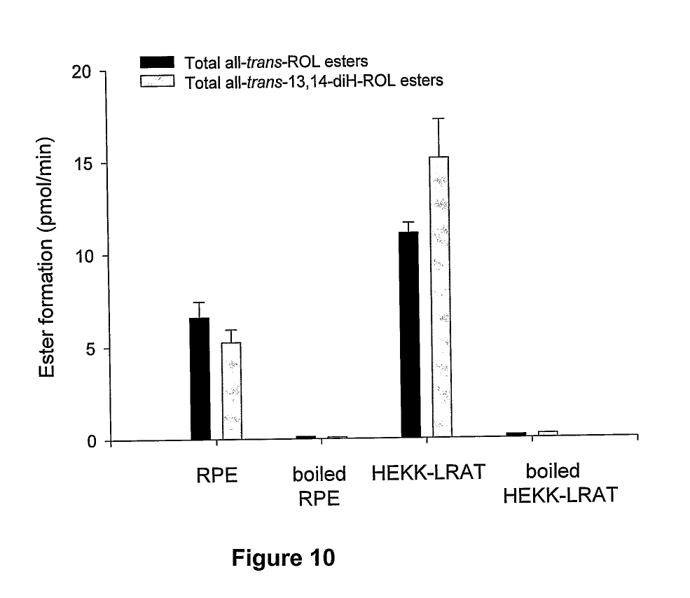

LRAT converts all-trans-retinol to all-trans-retinyl esters, thereby controlling its availability and absorption. Ruiz, et al. J Biol Chem 274:3834-3841, 1999; Batten, et al. J Biol Chem 279:10422-10432, 2004. To better understand the metabolism of all-trans-13,14-dihydroretinol we assayed whether it could be esterified by LRAT present in RPE or expressed in transfected cells according to previously published procedures. Kuksa, et al. Vision Res 43:2959-2981, 2003. We found that all-trans-13,14-dihydroretinol was as good a substrate for LRAT as all-trans-retinol by being converted to all-trans-13,14-dihydroretinyl esters (FIG. 10). This is in agreement with the esterification of all-trans-13,14-dihydroretinol by amphibian RPE. Law, et al. Journal of the American Chemical Society 110:5915-5917, 1988. From this we conclude that esterification may be a metabolic/storage pathway for all-trans-13,14-dihydroretinol, which will have to be confirmed in vivo.

FIG. 10 shows LRAT activity. Two nmol of retinols were incubated with RPE microsomes and with homogenized HEKK-LRAT cells for 10 min. The production of esters was monitored by HPLC measuring absorbance at 325 nm for all-trans-retinol (black bars) and 290 nm for all-trans-13,14-dihydroretinol (gray bars). Protein concentrations were not equalized. No activity was observed in controls with protein boiled for 10 min at 95° C. Experiments were performed in triplicate.

### EXAMPLE 10

RetSat Catalyzes the Saturation of the 13-14 Double Bond of all-trans-retinol

The findings presented in this report suggest that we uncovered a novel and potentially important pathway in the metabolism of vitamin A. RetSat, a novel enzyme, catalyzes a brand new activity, the saturation of the 13-14 double bond of all-trans-retinol. The product of the RetSat reaction, all-trans-13,14-dihydroretinol, was detected for the first time in vivo. The all-trans-13,14-dihydroretinol metabolite may be bioactive or may lead to other bioactive compounds; alternatively, it may be part of a catabolic pathway. Now that the enzyme and reaction have been identified, altering the activity of RetSat will allow us to investigate its role and that of all-trans-13,14-dihydroretinol in vivo.

Vertebrate (13,14)-all-trans-retinol saturase: an ancient enzyme. In addition to RetSat, enzymes involved in retinoid processing such as RALDH and CYP26 and one retinoic acid receptor (RAR) can be found in the translation of the draft genomic sequence of the primitive chordates, the ascidians Ciona intestinalis and Ciona savignyi. Dehal, et al. Science 298:2157-2167, 2002. The ascidian tadpole-larva contains a notochord and a dorsal tubular nerve cord much like a vertebrate tadpole and is considered a good approximation of the chordate ancestor. The acquisition of the anterioposterior organized body plan in chordates coincides with the innovation of RA and its nuclear receptor to control development. No RARs have so far been found in non-chordate species. Fujiwara, et al. Zoolog Sci 20:809-818, 2003. Identification of a putative ascidian RetSat underscores the potential importance of the pathway that starts with the saturation of the 13-14 double bond of retinol. As retinoid metabolism evolved, chordate metabolism modified an existing enzyme, possibly an ancient phytoene dehydrogenase, in order to create new metabolites with novel functions.

Carotenoid and retinoid-modifying enzymes share many features determined by the highly related nature of their substrates. The 9-cis-neoxanthin cleavage enzyme from plants, VP14, is similar to β,β-carotene-oxygenases BCO-I and -II from flies, ninaB, and vertebrates. Giuliano, et al. Trends Plant Sci 8:145-149, 2003. Another vertebrate protein related to carotenoid cleavage enzymes is RPE65, which is essential for the production of 11-cis-retinol, a key step of the visual cycle. Redmond, et al. Nat Genet 20:344-351, 1998. The function of RPE65 is not clear, as it was shown to bind retinyl esters, yet no catalytic role has been ascribed to it. Mata, et al. J Biol Chem 279:635-643, 2004. In vertebrates, P450 enzymes CYP26A1 and B1 convert retinoic acid (a diterpenoid) to hydroxylated metabolites. Fujii, et al. Embo J 16:4163-4173, 1997; White, et al. Proc Natl Acad Sci USA 97:6403-6408, 2000. Closely related P450 enzymes from plants hydroxylate abscisic acid, a sesquiterpene hormone that controls the plant life cycle, and taxol, a plant diterpenoid. Saito, et al. Plant Physiol 134:1439-1449, 2004; Kushiro, et al. Embo J 23:1647-1656, 2004. Based on its activity, RetSat is a retinoid-saturating enzyme related to carotenoid desaturases (phytoene desaturases Pds, Zds and CrtI), while the primary amino acid sequence relates to carotenoid isomerases, CRTISO.

### EXAMPLE 11

### Structural Analysis of the RetSat Enzyme.

Sequence analysis of the vertebrate RetSat family proteins reveals a dinucleotide-binding motif: U4G(G/A)GUXGL(X₂)(A/S)(X2)L(X₆₋₁₂)UX(L/V)UE(X4)UGG(X₉₋₁₃)(G/V)(X3)(D/E)XG where U is a hydrophobic residue and X is any residue. Wierenga, et al. J Mol Biol 187:101-107, 1986; Buehner, et al. J Mol Biol 82:563-585, 1974. Many proteins with this motif including monoamine oxidases, protoporphyrinogen oxidases, and many phytoene dehydrogenases have been shown to be stimulated by FAD and others or by NAD or NADP. Raisig, et al. J Biochem (Tokyo) 119:559-564, 1996; Al-Babili, et al. Plant J 9:601-612, 1996; Schneider, et al. Protein Expr Purif 10:175-179, 1997. The presence of a putative dinucleotide-binding motif in the sequence of RetSat argues that saturation of the double bond occurs through the transfer of a hydride (H—) ion from a reduced cofactor (NAD(P)H or FADH₂) and a proton from the solution. This may explain the labile nature of RetSat in homogenized cells, i.e., cells in which the redox state has been altered.

We show that mouse RetSat is membrane-associated and appears to localize to the ER compartment of transfected cells. A cleavable signal sequence can be readily identified at the amino terminus of the protein, indicating that the protein is targeted to the ER membrane. In addition, a stretch of hydrophobic amino acids from residue 568 to 588 is a strong candidate for a transmembrane domain.

### EXAMPLE 12

### 13,14-Dihydroretinols in Biological Systems

Retinoids containing saturated 13-14 double bonds such as 9-cis-13,14-dihydroretinoic acid and its taurine conjugate, 9-cis-4-oxo-13,14-dihydroretinoic acid were previously identified in animals supplemented with 9-cis-retinoic acid or retinyl palmitate, respectively. Shirley, et al. Drug Metab Dispos 24:293-302, 1996; Schmidt, et al. Biochim Biophys Acta 1583:237-251, 2002. Another saturated all-trans-13,14-dihydroxy-retinol was detected in retinol-treated lymphoblastoma 5/2 cells and was shown to support the proliferation of lymphocytes. Derguini, et al. J Biol Chem 270:18875-18880, 1995. The RetSat-catalyzed saturation reaction prefers all-trans-retinol as a substrate, which leads to specific synthesis of all-trans-13,14-dihydroretinol. Here we show that all-trans-13,14-dihydroretinol is detectable in unsupplemented animals (FIG. 9). It is preferable to demonstrate the existence of metabolite in vivo in animals maintained on a normal diet or receiving physiological levels of labeled precursor. This is the first report of this retinoid in vivo. Future studies will examine whether all-trans-13,14-dihydroretinol has biological activity or is metabolized to other active compounds. Though it is possible that all-trans-13,14-dihydroretinol is a breakdown product of all-trans-retinol, we find this unlikely since retinol and retinoic acid are degraded through oxidation to polar catabolites. The precise role of all-trans-13,14-dihydroretinol in vivo remains to be established, although it is involved in the metabolism of retinols.

### EXAMPLE 13

Relationship between Plant and Vertebrate Enzymes: a Productive Pathway of Discovery.

Carotenoids and retinoids play essential roles in biology. Their unique light-absorbing properties allow carotenoids to mediate photosynthesis and photoprotection and allow retinoids to form the visual chromophore. Through metabolites they can also regulate gene expression as seen for abscisic and retinoic acid. The only natural source of carotenoids, and hence retinoids, are plants and photosynthetic bacteria. Even though vertebrates do not synthesize carotenoids or retinoids, they are able to transform them to generate a unique series of metabolites. Vertebrate enzymes involved in carotenoid and retinoid processing probably evolved by substrate-switching an existing terpenoid modifying enzyme or by reactivating an ancestral gene inherited from a common ancestor of animals, plants, and photosynthetic bacteria. Studying the relationship between plant and vertebrate enzymes is a productive pathway of discovery. Both carotenoid and retinoid biochemistry can gain a new level of understanding through cross-fertilization of the two fields.

### EXAMPLE 14

### Materials and Methods

Metabolism of Retinoids in Vivo. All animal experiments employed procedures approved by the University of Washington and conformed to recommendations of the American Veterinary Medical Association Panel on Euthanasia and recommendations of the Association of Research for Vision and Ophthalmology. Animals were maintained on a 12-h light and 12-h dark cycle. All manipulations were done under dim red or infrared light (>560 nm). Most experiments used 6-12-week-old mice. Lrat−/− mice were genotyped as described previously. Batten, et al. J. Biol. Chem. 279:10422-10432, 2004. Animals were maintained on a control chow diet up to 1 h prior to oral gavage. The appropriate amount of all-trans-ROL palmitate, all-trans-DROL, or all-trans-RA was dissolved in vegetable oil and administered by oral gavage 3 h prior to analysis.

Analysis of Retinoids. Liver (1 g) from retinoid gavaged or naive mice was homogenized in 2 ml of 137 mM NaCl, 2.7 mM KCl, and 10 mM sodium phosphate (pH 7.4) for 30 s using a Polytron homogenizer. 10 μl of 5 M NaOH was added to 3 ml of the ethanolic extract, and the nonpolar retinoids were extracted using 5 ml of hexane. The extraction was repeated, and the organic phases were combined, dried under vacuum, resuspended in hexane, and examined by normal phase HPLC using a normal phase column (Beckman Ultrasphere Si 5μ, 4.6×250 mm). The elution condition was an isocratic solvent system of 10% ethyl acetate in hexane (v/v) for 25 min at a flow rate of 1.4 ml/min at 20° C. with detection at 325 and 290 nm for the detection of nonpolar retinoids and 13,14-dihydroretinoids, respectively. The aqueous phase was acidified with 40 μl of 12 N HCl, and polar retinoids were extracted with 5 ml of hexane. The extraction was repeated, and the organic phases of the polar retinoid extractions were combined, dried, resuspended in solvent composed of 80% CH₃CN, 10 mM ammonium acetate, 1% acetic acid, and examined by reverse phase HPLC. Analysis of polar retinoids from tissues was done by reverse phase HPLC using a narrowbore, 120-Å, 5-μm, 2.1×250 mm, Denali C18 column (Grace-Vydac, Hesperia, Calif.). The solvent system was composed of buffer A, 80% methanol, 20% 36 mM ammonium acetate (pH 4.7 adjusted with acetic acid), and buffer B, 100% methanol. The HPLC elution conditions were 0.3 mmin, 100% buffer A for 40 min, 100% buffer B for 10 min, and 10 min equilibration in buffer A. The elution profiles of RA and DRA were monitored using an online diode array detector set at 350 and 290 nm, respectively. The peaks were identified based on their UV-visible spectra and/or coelution with synthetic or commercially available standards. The measured area of absorbance was converted to picomoles based on a calibration of the HPLC columns using a known amount of all-trans-RA or all-trans-ROL (Sigma) and all-trans-DROL or all-trans-DRA (synthetic standards). The extraction efficiency was monitored by spiking a tissue sample with [³H]RA (PerkinElmer Life Sciences) and monitoring the radioactivity recovered from the HPLC column. In the case of liver samples the extraction efficiency was 95% or better. Mass spectrometry analyses of synthesized retinoids and of natural retinoids purified by HPLC were performed using a Kratos profile HV-3 direct probe mass spectrometer.

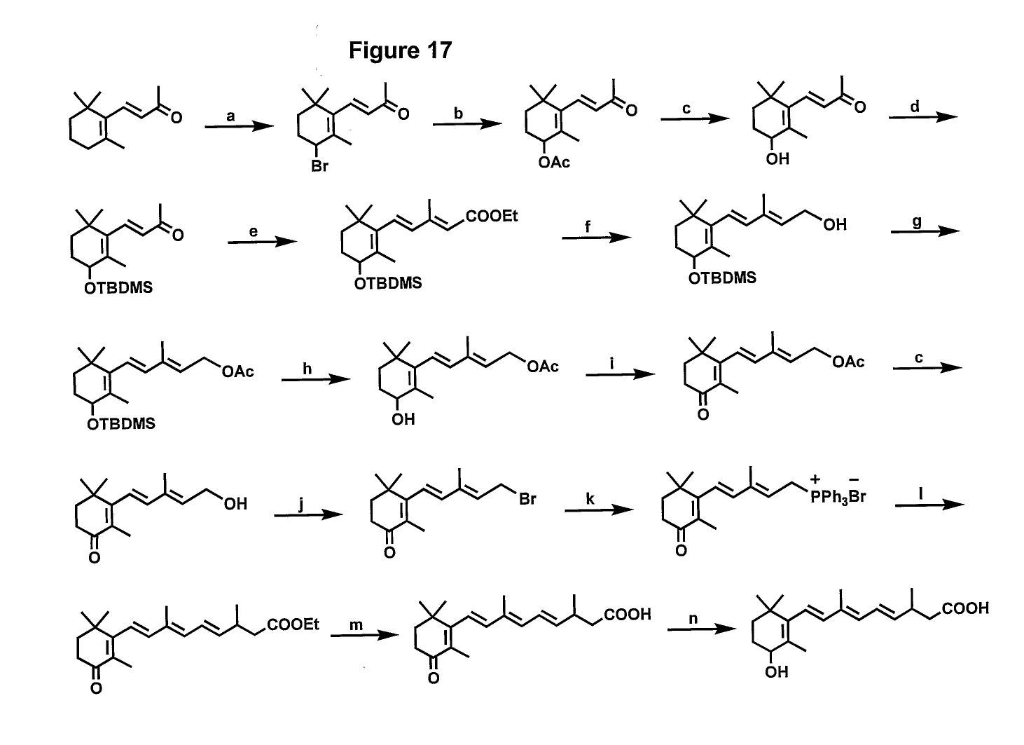

Synthesis and Analysis of 13,14-Dihydroretinoids. The synthetic scheme is depicted in FIG. 17. β-Ionone (I) was first brominated with N-bromosuccinimide in CCl₄ followed by substitution of bromine with an acetoxyl group in hexamethylphosphoramide. The acetate ester of ionone was hydrolyzed with K₂CO₃ in methanol:water, and then the hydroxyl group was protected with tetra-butyldimethylsilyl group. The silylated 4-hydroxy-ionone (II) was then condensed under Horner-Emmons conditions with triethylphosphonoacetate, and the ester of silyl-protected ethyl 4-hydroxy-β-ionylidene acetate was reduced to alcohol with LiAlH₄. The alcohol was acetylated with acetic anhydride in the presence of N,N-dimethylaminopyridine (DMAP); the silyl group was removed by tetrabutylammonium fluoride, and the alcohol was oxidized to a ketone group with MnO2 to give 15-acetoxy-4-oxo-β-ionylidene ethanol (III). Next, ester (III) was hydrolyzed, and the hydroxyl group was brominated with PBr₃ in ether. The bromide was reacted with PPh₃ to give Wittig salt (IV), which was further condensed with ethyl 4-oxo-3-methylbutyrate under conditions described previously to obtain a mixture of ethyl 13,14-dihydro-4-oxoretinoate isomers (V) with all-trans- as a major compound. Moise, et al. J. Biol. Chem. 279:50230-50242, 2004. The isomers were separated by normal phase HPLC (HP1100, Beckman Ultrasphere Si 5μ, 10×250 mm, 5% ethyl acetate:hexane, and detection at 325 nm) and characterized by their UV, mass, and NMR spectra. NMR data were recorded on a Bruker 500-MHz spectrometer using CDCl₃ as an internal standard, and their chemical shift values are listed in Table I. The order of delution was as follows: 9,11-di-cis-, all-trans-, 9-cis, 11-cis-13,14-dihydro-4-oxoretinoate. To obtain free retinoic acid (VI), the ethyl ester was hydrolyzed with NaOH in ethanol:H₂O. To obtain 13,14-dihydro-RAL (DRAL), previously prepared ethyl 13,14-dihydroretinoate was reduced with diisobutyl aluminum hydride at −78° C. All-trans-4-oxo-DRA has the following UV-visible absorbance spectrum in ethanol, λₘₐₓ=328 nm and shoulder λ=256 nm, and in hexane, λₘₐₓ=314 nm and shoulder at λ=252 nm.

Cloning and Expression Constructs. Total embryo and liver RNA was obtained from Ambion (Austin, Tex.) and reverse-transcribed using SuperScript II reverse transcriptase (Invitrogen) and oligo(dT) primers according to manufacturer's protocol. Embryo cDNA was used to amplify the cDNAs of specific genes using Hotstart Turbo Pfu polymerase (Stratagene, La Jolla, Calif.) and the following primers: RALDH1, forward 5′-CACCGCAATGTCTTCGCCTGCACAAC and reverse 5′-GCTGGCTTCTTTAGGAGTTCTTC; RALDH3 forward CACCTGCGAACCAGTTATGGCTACC and reverse 5′-GCCTGTTCCTCAGGGGTTCTT; CYP26B1, forward 5′-CACCAAGCGGCTGCCAACATGC and reverse 5′-GCTGAGACCAGAGTGAGGCTA; and CYP26C1 forward 5′-CACCCATTCTCGCCATGATTTCCT and reverse 5′-CCAAGGCTAGAGAAGCAACG. The full-length cDNA of RALDH2 (MGC:76772, IMAGE: 30471325), RALDH4 (MGC:46977, IMAGE:4223059), and CYP26A1 (MGC:13860, IMAGE:4210893) mRNA was obtained from the Mammalian Gene Collection (MGC). These clones were used as templates to amplify the respective cDNAs using Hotstart Turbo Pfu polymerase (Stratagene) and the following primers: RALDH2, forward 5′-CACCATGGCCTCGCTGCAGCTCCTGC and reverse 5′-GGAGTTCTTCTGGGGGATCTTCA; RALDH4, forward 5′-CACCTGTACACAGAGGGCACTTTCC and reverse 5′-GTATTTAATGGTAATGGTTTTTATTTCAGTAAAG; and CYP26A1, forward 5′-CACCATGGGGCTCCCGGCGCTGCT and 5′-GATATCTCCCTGGAAGTGGGTAAAT. The cDNAs for RALDH1, -2, -3, and -4 and CYP26A1, -B1, and -C1 were cloned in the pcDNA3.1 Directional TOPO vector under the control of the CMV promoter to express a recombinant protein fused with a C-terminal V5 epitope peptide (GKPIPNPLLGLDST) and a His₆ tag (Invitrogen). Both strands of the expression constructs were sequenced to ensure no mutations were present.

Mouse RXR-α was cloned using the primers 5′-GGGCATGAGTTAGTCGCAGA and 5′-AGCTGAGCAGCTGTGTCCA from reverse-transcribed mouse liver cDNA. The RXR-α open reading frame was then subcloned into the pcDNA3.1 Directional TOPO vector (Invitrogen) using the primers 5′-CACCATGGACACCAAACATTTCCT and 5′-AGCTGAGCAGCTGTGTCCA under the control of the CMV promoter. The RXRE from the vector RXR (2) translucent reporter vector (Panomics, Redwood City, Calif.) was amplified using the primers 5′-CTCAACCCTATCTCGGTCTATTCT and 5′-ATGCCAGCTTCATTATATACCCA and cloned upstream of the minimal promoter and β-galactosidase open reading frame of pBLUE-TOPO (Invitrogen) to create the pRXRE-BLUE expression construct. This construct places five consecutive DR1 elements upstream of β-galactosidase, the expression of which becomes dependent on activation of RXR and formation of RXR homodimers. Both strands of all constructs were sequenced to ensure no mutations were present.

Oxidation of All-trans-ROL and All-trans-DROL Using Liver Alcohol Dehydrogenase. Equine liver ADH (EC 1.1.1.1 [EC] ) was obtained from Sigma and dissolved in 50 mM Tris (pH 8.8) to a concentration of 5 units/ml (8.6 mg/ml). NAD and NADP were mixed together (1:1) at a concentration of 10 mM each. A substrate solution, 2 μl of 2 mM stock of all-trans-ROL or all-trans-DROL in N,N-dimethylformamide, was added to a 1.5-ml Eppendorf tube containing 20 μl of 10% bovine serum albumin, 20 μl of ADH, 2 μl of cofactor mixture, and 50 mM Tris (pH 8.8) to a total volume of 200 μl. The solutions were incubated at 37° C. for 60 min, after which 50 μl of 0.8 M NH₂OH solution (pH 7.0) was added, followed by addition of 300 μl of methanol, 15 min at room temperature, and extraction with 300 μl of hexane. The organic phase was dried and analyzed by normal phase HPLC as described in the analysis of nonpolar retinoids extracted from tissue samples. As a control for the nonenzymatic reaction, boiled protein (90° C. for 5 min) was used with or without addition of cofactors.

RALDH Oxidation Assay. N-Acetylglucosaminyltransferase I-negative HEK-293S cells, obtained from Dr. G. Khorana (Massachusetts Institute of Technology, Boston) were cultured in Dulbecco's modified Eagle's medium, 10% fetal calf serum and maintained at 37° C., 5% CO₂, and 100% humidity. Reeves, et al. Proc. Natl. Acad. Sci. U.S.A. 99:13419-13424, 2002. For RALDH enzyme assays, cells were transiently transfected with RALDH1, -2, -3, or -4 expression constructs using Lipofectamine 2000 (Invitrogen) according to the manufacturer's protocol. After 48 h post-transfection, the cells were collected by scraping and were centrifuged. The cell pellet was washed in 137 mM NaCl, 2.7 mM KCl, and 10 mM phosphate (pH 7.4), resuspended in 50 mM Tris (pH 8.0) containing 250 mM sucrose, and homogenized with the aid of a Dounce homogenizer. Cofactors were added to a final concentration of 5 mM NAD, 5 mM NADP, and 1 mM ATP. An aliquot of the cell lysate was boiled for 10 min at 95° C. to provide the control for the nonenzymatic reaction. Substrates in the form of all-trans-RAL or a mixture of isomers of DRAL were added to the cell lysates at a final concentration of 60 μM. The reactions were allowed to proceed for 2 h at 37° C. with shaking and were stopped by the addition of 2 volumes of CH₃CN. Samples were treated for 30 min at room temperature with 100 mM NH2OH (final concentration from a freshly made stock of 1 M (pH 7.0)) followed by centrifugation at 12,000×g for 10 min. The clear supernatant was acidified with 0.1 volume of 0.5 M ammonium acetate (pH 4.0) and examined by reverse phase HPLC system (Zorbax ODS, 5 μm, 4.6×250 nm; Agilent, Foster City, Calif.) with an isocratic mobile phase A of 80% CH₃CN, 10 mM ammonium acetate, 1% acetic acid, and a flow rate of 1.6 ml/min held for 15 min. After each run, the column was washed with mixture B (60% tert-butylmethyl ether, 40% methanol) for 10 min at 1.6 ml/min, followed by re-equilibration in phase A. The elution of RA and DRA isomers was monitored at 340 and 290 nm, respectively. The peaks were identified based on their spectra and coelution with standards. The cell lysate was examined for expression of RALDH1-4 by SDS-PAGE and immunoblotting of the V5 epitope-tagged recombinant protein using an anti-V5 epitope monoclonal antibody (Invitrogen).

CYP26A1 Oxidation Assay. N-Acetylglucosaminyltransferase I-negative HEK-293S cells were transiently transfected with cDNAs of CYP26A1, -B1, and -C1 under the control of CMV promoter using Lipofectamine 2000 (Invitrogen) according to the manufacturer's protocol. After 24 h, the transfected cells were split into 12-well plates to ensure an equal number of transfected cells in each assay well. All-trans-RA or all-trans-DRA was added to the cell monolayer at 0.1 mM final concentration in complete media and incubated for 4 h. Media and cells were collected by scraping, and proteins were precipitated with an equal amount of CH3CN by vigorous vortexing followed by centrifugation at 12,000×g for 10 min. For RA analysis the clear supernatant was acidified with 0.1 volume of 0.5 M ammonium acetate (pH 4.0) and examined by reverse phase HPLC as described for the RALDH assays. The elution of all-trans-RA, all-trans-DRA, and their oxidized metabolites was monitored at 340 and 290 nm. The peaks were identified based on their spectra and coelution with standards. The cell lysate was examined for expression of CYP26A1, -B1, and -C1 by SDS-PAGE and immunoblotting of the V5 epitope-tagged recombinant protein using an anti-V5 epitope monoclonal antibody (Invitrogen).

Conversion of DROL to DRA in RPE. UV-treated RPE microsomes were prepared as described previously. Stecher, et al. J. Biol. Chem. 274:8577-8585, 1999. Twenty μl of UV-treated RPE microsomes (3 mg/ml) were mixed with 20 μM DROL or ROL substrates, 1% bovine serum albumin, and 50 mM Tris (pH 8.8) and were incubated at 37° C. for 60 min in the presence or absence of NAD NADP cofactor mixture at 50 μM each. In order to stop the reaction, proteins were precipitated by mixing with an equal volume of CH3CN followed by high speed centrifugation. The clear supernatant was acidified with 0.1 volume of 0.5 M ammonium acetate (pH 4.0) and examined by reverse phase HPLC as described for the RALDH assays. A boiled RPE membrane control was used to assay nonenzymatic conversion of DROL. The elution of all-trans-DROL metabolites was monitored at 290 nm.

RARE and RXRE Activation Assay. The RARE reporter cell line F9-RARE-lacZ (SIL15-RA) was a kind gift from Dr. Michael Wagner (State University of New York Downstate Medical Center) and Dr. Peter McCaffery (University of Massachusetts Medical School, E. K. Shriver Center). The RA-responsive F9 cell line was transfected with a reporter construct of an RARE derived from the human retinoic acid receptor-β gene (RAR-β) placed upstream of the Escherichia coli lacZ gene. Wagner, et al. Development (Camb.) 116:55-66, 1992. Cells were grown in L15-CO₂ media containing N-3 supplements and antibiotics. Cells were stimulated for 24 h in the dark at 37° C. and 100% humidity with all-trans-RA or all-trans-DRA dissolved in ethanol at the indicated concentrations, lysed, and assayed for the expression of -galactosidase using the -galactosidase enzyme assay system (Promega, Madison Wis.). For RXRE activation assays N-acetylglucosaminyltransferase I-negative HEK-293S cells were transfected with the pRXRE-BLUE reporter construct with or without the RXR-expression construct using Lipofectamine 2000 (Invitrogen) according to the manufacturer's protocol. After 24 h, cells were split into 24-well plates to ensure an equal number of transfected cells in each assay well. Cells were stimulated with appropriate concentrations of all-trans-RA, 9-cis-RA, or all-trans-DRA. After 48 h, the expression of β-galactosidase was assayed as described above.

### EXAMPLE 15

Identification of All-trans-DROL and its Metabolites in the Liver of Lrat−/− Mice Gavaged with All-trans-ROL Palmitate

ROL absorption in mammals is an active process driven by esterification and hydrolysis cycles. Esterification of ROL is carried out mainly by the LRAT enzyme. Ruiz, et al. J. Biol. Chem. 274:3834-3841, 1999. In the absence of LRAT, the equilibrium between ROL and ROL esters is shifted in favor of free ROL. Mice deficient in LRAT expression (Lrat−/−) mice are severely impaired in their ROL uptake and storage capacity. Batten, et al. J. Biol. Chem. 279:10422-10432, 2004. Wild type mice, on the other hand, convert most of the ingested ROL to esters, which sequester ROL from circulation and metabolism. Thus, we chose to study the saturation and oxidation of all-trans-ROL to 13,14-dihydroretinoid metabolites in Lrat−/− mice.

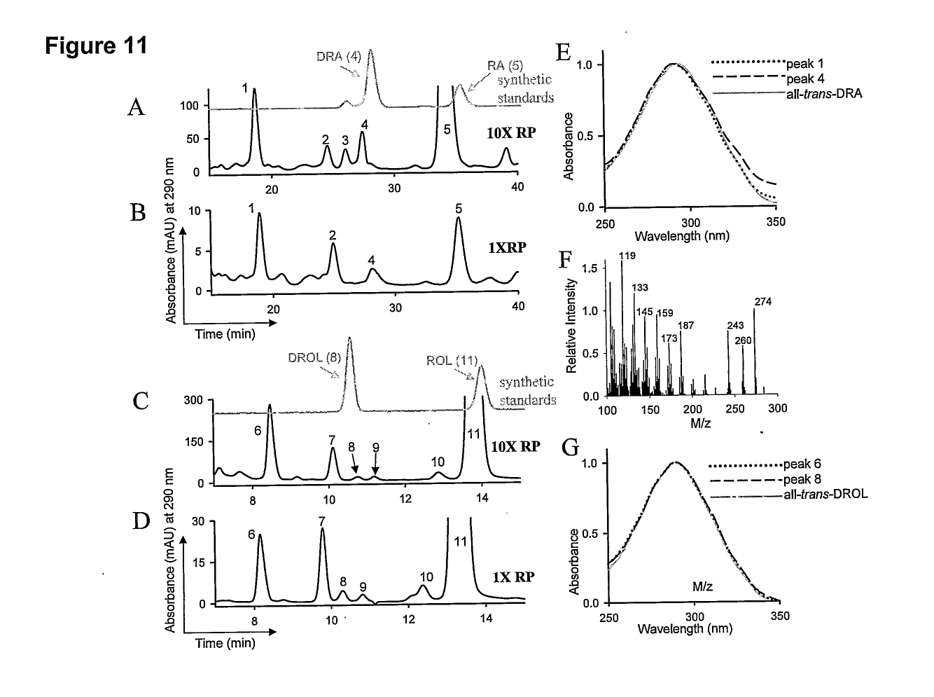

Given their similar chemical properties, it is not surprising that all-trans-DROL and all-trans-ROL follow parallel metabolic pathways. Two different groups of Lrat−/− mice were dosed with either 106 units of all-trans-ROL palmitate/kg body weight or 105 units of all-trans-ROL palmitate/kg body weight, and their livers were examined for polar and nonpolar retinoid metabolites at 3 h post-gavage. Reverse phase HPLC analysis of polar hepatic retinoids indicated the presence of all-trans-RA (FIGS. 11, A and B, peak 5) and all-trans-DRA (FIGS. 11, A and B, peak 4), as well as a cis-DRA isomer (FIGS. 11, A and B, peak 2). We also observed another polar DROL metabolite, which eluted earlier than all-trans-DRA, on reverse phase HPLC (FIGS. 11, A and B, peak 1) and had the same absorbance spectrum as all-trans-DRA standard (FIG. 11E). This metabolite was not chemically characterized; however, based on its polar character, it could represent a taurine or glucuronide DRA conjugate. The spectra and elution profiles of synthetic all-trans-DRA and all-trans-DRA isolated from liver matched (FIG. 11E). All-trans-DRA was synthesized according to procedures published previously and was characterized by 1H NMR (Table I). Moise, et al. J. Biol. Chem. 279:50230-50242, 2004.

TABLE I
[¹H]-NMR chemical shift values of relevant 13,14-dihydroretinoids.

| Dihydroretinoid, | | | | | | | | | | | | | | | | |
|---|---|---|---|---|---|---|---|---|---|---|---|---|---|---|---|---|
| CDCl₃, 500 MHz | H-2 | H-3 | H-4 | H-7 | H-8 | H-10 | H-11 | H-12 | H-13 | H-14 | H-15 | H-16, 17 | H-18 | H-19 | H-20 | Et |
| Et-9-cis-DRA, ppm | 1.47 | 1.63 | 2.03 | 6.13 | 6.56 | 5.88 | 6.51 | 5.56 | 2.76 | 2.31 | N/A | 1.02 | 1.91 | 1.73 | 1.08 | 4.12, 1.24 |
| Hz | | | 5.9 | 16.0 | 16.0 | 11.2 | 14.9 | 14.9 | 6.7 | | | | | | 6.7 | 7.0, 7.0 |
| Hz | | | | | | | 11.2 | 7.6 | 7.3 | | | | | | | |
| Et-9,11-dicis-DRA, ppm | 1.46 | 1.61 | 2.01 | 6.18 | 6.60 | 6.21 | 6.41 | 5.20 | 3.21 | 2.29 | N/A | 1.02 | 1.96 | 1.71 | 1.05 | 4.10, 1.24 |
| Hz | | | 6.2 | 16.0 | 16.0 | 16.2 | 11.0 | 10.4 | | | | | | 6.7 | | 7.3 |
| Hz | | | | | | | | | | | | | | | | |
| Et-all-trans-DRA, ppm | 1.46 | 1.60 | 2.00 | 6.11 | 6.04 | 5.98 | 6.42 | 5.63 | 2.78 | 2.32 | N/A | 1.00 | 1.89 | 1.69 | 1.09 | 4.13, 1.25 |
| Hz | | | 6.4 | 16.1 | 16.1 | 10.8 | 15.1 | 15.1 | 7.0 | | | | | | 6.6 | 6.8, 7.0 |
| Hz | | | | | | | 11.4 | 7.7 | | | | | | | | |
| Et-11-cis-DRA, ppm | 1.46 | 1.61 | 2.01 | 6.14 | 6.14 | 6.31 | 6.31 | 5.27 | 3.21 | 2.30 | N/A | 1.02 | 1.90 | 1.71 | 1.05 | 4.09, 1.23 |
| Hz | | | | | | | | 8.6 | | | | | | | 6.7 | 7.2, 7.3 |
| Hz | | | | | | | | | | | | | | | | |
| Et-all-trans-DORA, ppm | 1.84 | 2.49 | N/A | 6.17 | 6.26 | 6.10 | 6.43 | 5.73 | 2.80 | 2.34 | N/A | 1.16 | 1.83 | 1.92 | 1.09 | |
| Hz | 6.9 | 6.9 | | 16.2 | 16.2 | 11.1 | 12.2 | 15.0 | 7.6 | | | | | | 7.2 | |
| Hz | | | | | | | 11.1 | 7.6 | | | | | | | | |
| Et-11-cis-DORA, ppm | 1.85 | 2.50 | N/A | 6.22 | 6.33 | 6.43 | 6.33 | 5.38 | 3.21 | 2.30 | N/A | 1.20 | 1.85 | 1.92 | 1.05 | |
| Hz | | 6.8 | | 16.2 | | 11.8 | | 10.4 | | | | | | | 6.9 | |
| Hz | | | | | | | | 10.1 | | | | | | | | |
| Et-9-cis-DORA, ppm | 1.86 | 2.52 | N/A | 6.20 | 6.78 | 6.04 | 6.47 | 5.66 | 2.77 | 2.32 | N/A | 1.18 | 1.87 | 1.94 | 1.08 | |
| Hz | 6.9 | 6.9 | | 16.1 | 16.1 | 10.8 | 14.5 | 7.8 | 7.3 | | | | | | 7.0 | |
| Hz | | | | | | | 10.9 | 15.3 | | | | | | | | |
| Et-9,11-dicis-DORA, | 1.83 | 2.48 | N/A | 6.22 | 6.79 | 6.35 | 6.35 | 5.27 | 3.19 | 2.28 | N/A | 1.15 | 1.83 | 1.97 | 1.04 | |
| ppm | | | | | | | | | | | | | | | | |
| Hz | | 6.6 | | 16.1 | 16.1 | | | 9.5 | | | | | | | 6.7 | |
| Hz | | | | | | | | | | | | | | | | |
| All-trans-DROL, ppm | 1.46 | 1.61 | 2.00 | 6.11 | 6.05 | 5.99 | 6.41 | 5.60 | 2.41 | 1.61 | 3.67 | 1.01 | 1.90 | 1.69 | 1.06 | |
| Hz | | | 6.1 | 16.2 | 16.2 | 11.2 | 14.9 | 15.3 | 7.6 | | | | | | 6.8 | |
| Hz | | | | | | | 11.0 | 8.5 | | | | | | | | |
| 11-cis-DROL, ppm | 1.46 | 1.61 | 2.01 | 6.16 | 6.10 | 6.28 | 6.33 | 5.27 | 2.86 | 1.66 | 3.63 | 1.02 | 1.91 | 1.71 | 1.03 | |
| Hz | | | | 16.0 | 16.0 | 11.9 | 11.5 | 10.1 | | | | | | | | |
| Hz | | | | | | | 10.7 | | | | | | | | | |

NMR data were recorded on a Bruker 500-MHz spectrometer using CDCl3 as an internal standard.

We examined the nonpolar hepatic retinoid metabolites by normal phase HPLC. At 3 h post-gavage with ROL palmitate, the livers of the examined mice contained high levels of all-trans-ROL (FIGS. 11, C and D, peak 11), whereas all-trans-DROL (FIGS. 11, C and D, peak 8) was found at 280-330-fold lower levels (Table II). The absorbance spectra and elution profile of all-trans-DROL matched the synthetic standard prepared according to published procedures and characterized by 1H NMR (FIGS. 11, C, D, and G, and Table I). Moise, et al. J. Biol. Chem. 279:50230-50242, 2004.

TABLE II
Level of liver retinoids 3 hr following gavage with ROL palmitate^{a}.

| | 10⁶ IU/kg body | 10⁵ IU/kg body |
|---|---|---|
| | weight-dose | weight-dose |
| | level of all-trans-ROL | level of all-trans-ROL |
| | palmitate | palmitate |
| Compound identified | (pmol/g tissue) | (pmol/g tissue) |
| all-trans-RA | 9,400 ± 300 | 320 ± 240 |
| all-trans-DRA | 190 ± 17 | 10 ± 2 |
| cis-DRA | 180 ± 53 | 22 ± 4 |
| Peak 1 FIG. 11 A & B | 460 ± 50 | 37 ± 9 |
| all-trans-ROL | 28,000 ± 300 | 7,000 ± 1,200 |
| all-trans-DROL | 100 ± 18 | 21 ± 4 |
| Peak 6 FIG. 11 C & D | 1,800 ± 370 | 200 ± 8 |

^{a}The analysis was carried out as described in the Materials and Methods.

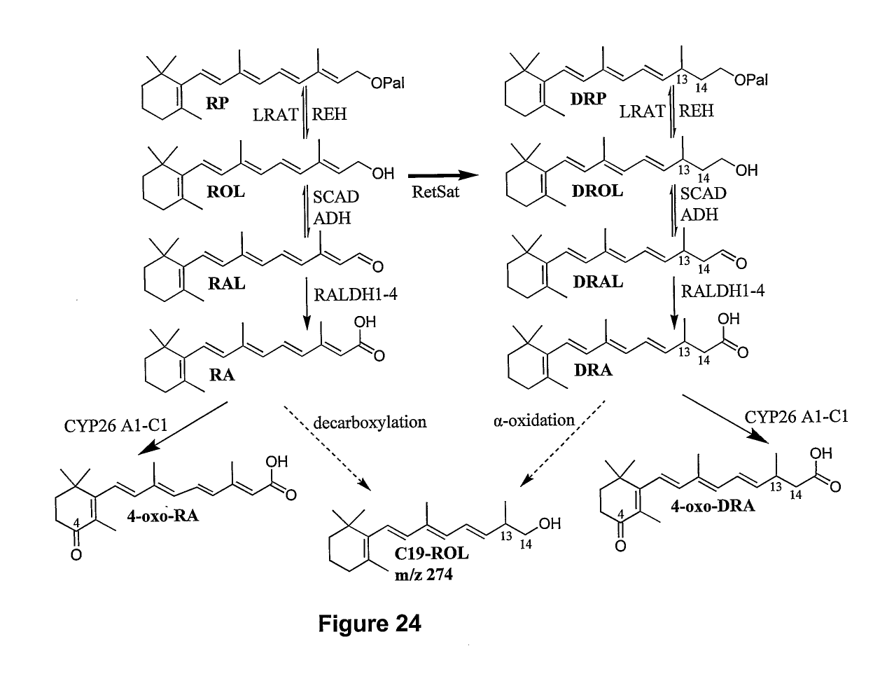

Another nonpolar 13,14-dihydroretinoid metabolite (FIGS. 11, C and D, peak 6) that was present at higher levels than DROL was identified in the liver of mice gavaged with all-trans-ROL palmitate. The spectra of this compound also matched that of all-trans-DROL (FIG. 11G). The compound does not coelute with cis-DROL isomers and has a different UV-visible absorbance maximum than cis-DROL isomers. We were able to esterify the compound, whereas NH2OH treatment had no effect on its elution profile . Thus, we conclude that the functional group of the compound eluting as peak 6 (FIGS. 11, C and D) is alcohol. Electron-impact mass spectrometry analysis of the collected fraction corresponding to peak 6 indicates the presence of a compound with an m/z of 274 (FIG. 11F). This suggests that peak 6 could include the chain-shortened C19-ROL derivative (C₁₉H₃₀O, m/z=274, depicted in FIG. 24).

FIG. 11 shows the analysis of metabolism of all-trans-ROL palmitate in the liver of Lrat−/− mice. A-D, HPLC analysis of the polar and nonpolar retinoids from the liver of Lrat−/− mice gavaged with all-trans-ROL palmitate. Mice were gavaged with all-trans-ROL palmitate at a high dose of 10⁶ units/kg body weight (marked as 10XRP, n=3) in A and C or with a lower dose of all-trans-ROL palmitate of 10⁵ units/kg body weight (marked as 1XRP, n=3) in B and D. Three h after gavage, the polar and nonpolar retinoids from liver were extracted. The retinoids were analyzed by reverse phase HPLC on a narrowbore column system (A and B), and the nonpolar retinoids were analyzed by normal phase HPLC (C and D). Compounds were identified based on comparison with the elution profile and absorbance spectra of authentic standards. E, the spectrum of peak 4 matches that of all-trans-DRA standard, with which it coelutes. The absorbance spectrum of another compound, peak 1, eluting earlier than all-trans-DRA by reverse phase HPLC, also matches that of all-trans-DRA. F, the electron impact mass spectrometry analysis of the compound eluting as peak 6 in C and D indicates it is a possible mixture of compounds with m/z of 274 and 260. G, the compound eluting as peak 6 exhibits a UV-visible absorbance profile identical to the one of biological all-trans-DROL (peak 8) and of synthetic all-trans-DROL. Elution of all-trans-RA was monitored at 350 nm, all-trans-ROL at 325 nm, and all-trans-DROL and all-trans-DRA at 290 nm. Only the absorbance at 290 nm is shown here for simplicity. The extraction efficiency was >95% and was calculated based on spiking samples with [³H]RA and measuring the radioactivity associated with the RA peak. Based on elution time, absorbance spectra, and comparison with authentic standards, the peaks were identified as the following compounds: peak 2, cis-DRA; peak 3, 13-cis-RA; peak 4, all-trans-DRA; peak 5, all-trans-RA; peak 6, C19-ROL derivative; peak 7, 13-cis-ROL; peak 8, all-trans-DROL; peak 9, 9,13-di-cis-ROL; peak 10, 9-cis-ROL; and peak 11, all-trans-ROL.

FIG. 24 shows the metabolism of all-trans-ROL and all-trans-DROL. RetSat saturates all-trans-ROL to all-trans-DROL, which was previously shown to be esterified by LRAT. Here we present evidence demonstrating that the oxidative metabolism of DROL closely follows that of ROL. Broad spectrum enzymes such as SDR and ADH carry out the reversible oxidation of all-trans-DROL to all-trans-DRAL. RALDH1, -2, -3, and -4 oxidize all-trans-DRAL to all-trans-DRA. Several members of the cytochrome P450 enzymes CYP26A1, -B1, and -C1 oxidize all-trans-DRA to all-trans-4-oxo-DRA, identified in vivo and in vitro. Other oxidized all-trans-DRA metabolites, which are not depicted, could be all-trans-4-hydroxy-DRA, all-trans-5,6-epoxy-DRA, all-trans-5,8-epoxy-DRA, and all-trans-18-hydroxy-DRA. The short-chain metabolite C19-ROL is shown here with its possible chemical structure. Its synthetic pathway may proceed from either all-trans-RA by decarboxylation and/or from all-trans-DRA via α-oxidation.

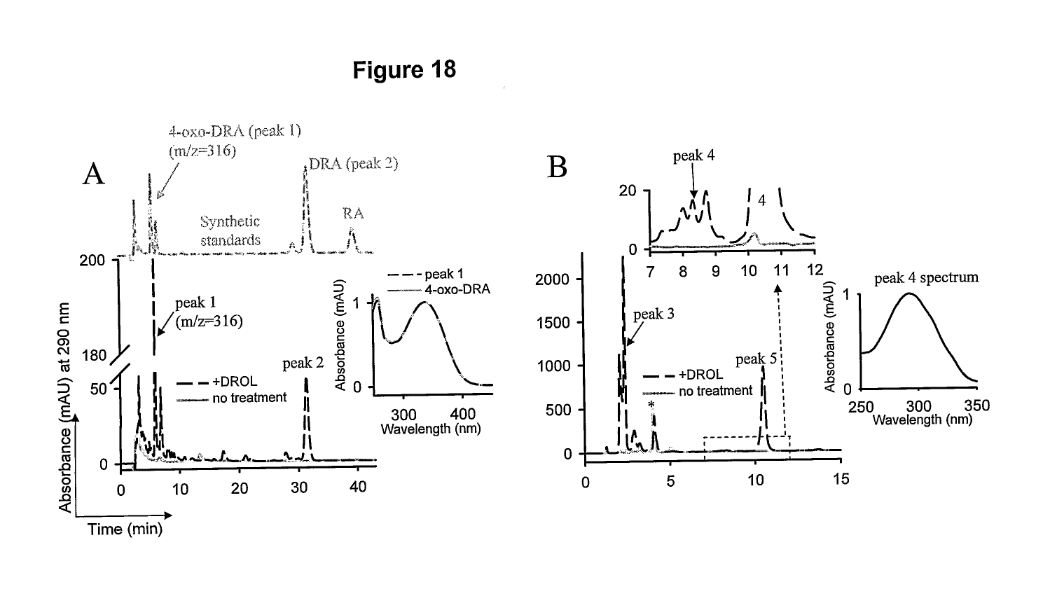

Following gavage of Lrat−/− mice with synthetic all-trans-DROL, we observed significant levels of all-trans-DRA and all-trans-4-oxo-DRA. These were identified based on their chromatographic profile, m/z, and absorbance spectra, which matched those of synthetic standards (FIG. 18A and inset spectra). All-trans-4-oxo-DRA was synthesized according to the scheme depicted in FIG. 17 and was characterized by ¹H NMR (Table I). The livers of mice gavaged with DROL were also found to contain low levels of C19-ROL (FIG. 18B, peak 4, and inset spectrum). This is in contrast to the high levels of C19-ROL observed in all-trans-ROL palmitate gavaged mice.

FIG. 17 shows compound all-trans-4-oxo-DRA (VI) was characterized by [1H]-NMR. Synthetic scheme for the preparation of 4-oxo-DRA and 4-hydroxy-DRA: a, NBS, (PhCOO)2, CC14, reflux, 20 min; b, KOAc, HMPA, room temperature, 24 hr; c, K2CO3, MeOH:H2O, room temperature, 6 hr; d, TBDMS-Cl, CH2Cl2, DMAP, room temperature, 18 hr; e, (EtO)2P(O)CH2COOEt, NaH, THF, reflux, 24 hr; f, LiAliH4, Et2O, 0° C., 30 min; g, Ac2O, DMAP, CH2Cl2, room temperature, 2 hr; h, TBAF, THF, room temperature, 16 hr; i, MnO2, CH2Cl2, room temperature, 24 hr; j, PBr3, Py, Et2O, −20° C., 1 hr; k, PPh3, toluene, room temperature, 24 hr; 1, t-BuO-K+, 18-crown-6, CH2Cl2, −78° C. to room temperature, 6 hr; m, 5 M NaOH, EtOH/H2O, 37° C., 1 hr; n, NaBH4, EtOH, 0° C., 30 min.

FIG. 18 shows analysis of metabolism of all-trans-DROL in the liver of Lrat−/− mice. HPLC analysis of the polar and non-polar retinoids from the liver of Lrat−/− mice gavaged with all-trans-DROL (n=3). Three hr after gavage of Lrat−/− mice with all-trans-DROL, the polar and non-polar retinoids were extracted and analyzed by reverse-phase HPLC (A, black dashed line chromatogram) or normal-phase HPLC (B, black dashed line chromatogram). Synthetic standards all-trans-4-oxo-DRA, all-trans-DRA, and all-trans-RA were examined by reverse-phase HPLC (A, top chromatogram, gray dashed line). Hepatic retinoids isolated from control unsupplemented Lrat−/− mice were also examined (A and B gray solid line chromatograms). (B) The 7 to 12 min area of the chromatogram was expanded to indicate peak 4, attributed to C19-ROL. Peak 3 consisted of a mixture of ester compounds including all-trans-DROL-decanoate (m/z=442) and all-trans-DROL-palmitate (m/z=526) as determined by electron-impact mass spectrometry. The following compounds were identified based on their elution profile, absorbance spectra, and comparison with synthetic standards: (1), all-trans-4-oxo-DRA and (A) inset spectrum; (2), all-trans-DRA; (3), all-trans-DROL esters; (4), C19-ROL and (B) inset spectrum; (5) all-trans-DROL. Compounds that could not be identified are indicated with an asterisk (*).

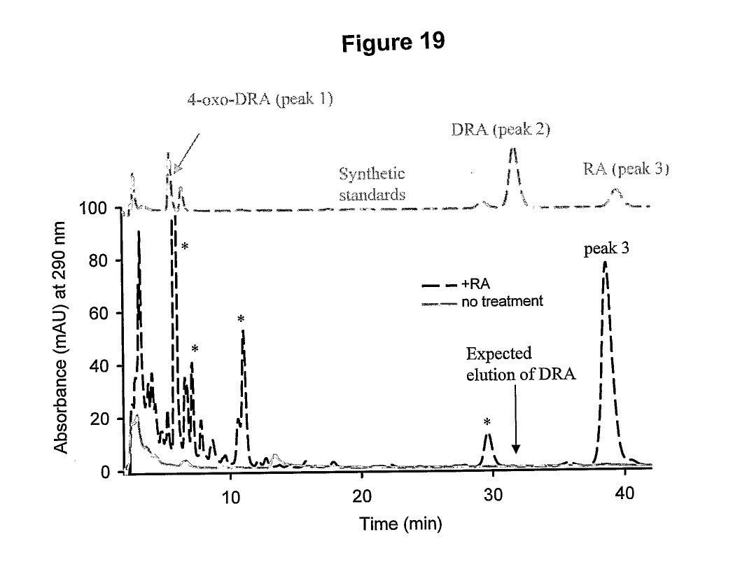

It has been reported that rats can convert exogenously administered 9-cis-RA to 9-cis-DRA and its taurine conjugate. Shirley, et al. Drug Metab. Dispos. 24:293-302, 1996. We have shown that RetSat does not saturate all-trans-RA or 9-cis-RA. Moise, et al. J. Biol. Chem. 279:50230-50242, 2004 This would suggest that another pathway is responsible for saturation of the C₁₃₋₁₄ bond of RA to produce DRA. In the current study, we found no evidence of all-trans-DRA or all-trans-4-oxo-DRA formation in the livers of Lrat−/− mice gavaged with all-trans-RA at 3 h post-gavage (FIG. 19). A compound different from all-trans-DRA (FIG. 19, marked with *) with a maximum absorbance of 257 nm eluted before the expected elution time of all-trans-DRA. This would suggest that 13,14-dihydroretinoid metabolites can only be derived from all-trans-DROL after saturation of all-trans-ROL by RetSat, emphasizing the key role played by RetSat at this branch of vitamin A metabolism. We also found no evidence of C19-ROL in the livers of Lrat−/− mice gavaged with all-trans-RA at 3 h post-gavage.

FIG. 19 shows analysis of metabolism of all-trans-RA in the liver of Lrat−/− mice. Reverse-phase HPLC analysis of the polar retinoids from the liver of Lrat−/− mice 3 hr post-gavage with all-trans-RA (black dashed line chromatogram). Synthetic standards all-trans-4-oxo-DRA, all-trans-DRA, and all-trans-RA were examined by reverse-phase HPLC (top chromatogram, gray dashed line). Hepatic retinoids isolated from control unsupplemented Lrat−/− mice were also examined (gray solid line). Compounds that could not be identified are indicated with an asterisk (*).

The levels of all-trans-RA, all-trans-DRA, and the compounds eluting as peak 1 in FIGS. 11, A and B, and as peak 6 in C and D, are indicated in Table II and reflect the different starting levels of ingested ROL palmitate. The levels of all-trans-DRA are much lower (30-50-fold) than those of all-trans-RA, which could indicate that saturation by RetSat is a limiting step. The low levels of all-trans-DROL in comparison with all-trans-ROL also support this explanation. The levels of all-trans-DROL and all-trans-DRA may also be low because of further processing to shorter chain or to other more oxidized metabolites.

### EXAMPLE 16

### Characterization of the Metabolic Pathway of All-trans-DROL to All-trans-DRA

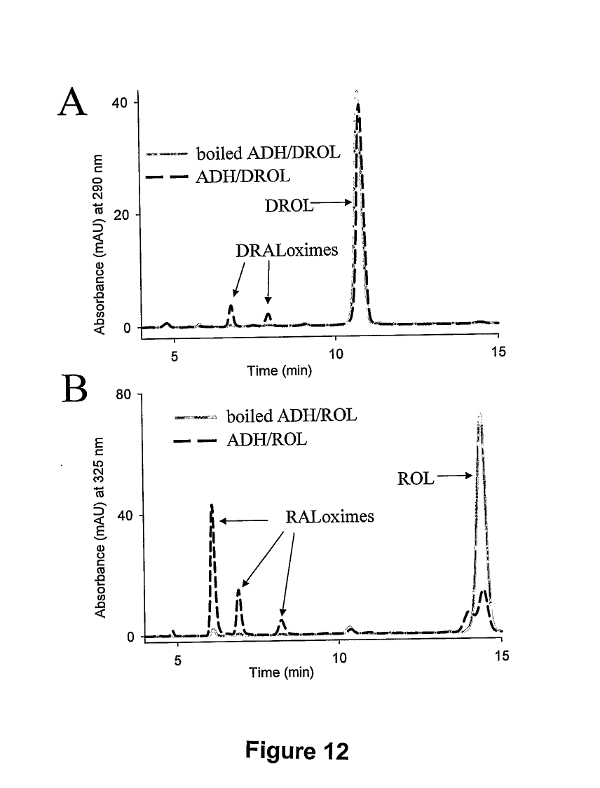

Given that all-trans-DRA is detected in vivo as a metabolite of all-trans-DROL, we decided to examine its possible mode of synthesis using reconstituted enzyme systems. To oxidize all-trans-DROL to the corresponding aldehyde all-trans-DRAL, we used ADH purified from horse liver (EC 1.1.1.1 [EC]), which is active toward both primary and secondary alcohols. All-trans-DROL and all-trans-ROL were incubated with purified enzyme and the appropriate cofactors. Following the reaction the samples were treated with NH2OH, extracted into the organic phase, and examined by normal phase HPLC. All-trans-RAL or all-trans-DRAL oximes were identified by comparison with synthetic standards. Moise, et al. J. Biol. Chem. 279:50230-50242, 2004. ADH efficiently carried out the conversion of all-trans-ROL to all-trans-RAL and of all-trans-DROL to all-trans-DRAL in the presence of NAD and NADP cofactors (FIGS. 12, A and B) and not in their absence. The boiled enzyme did not exhibit any activity toward either substrate. Next, photoreceptor-specific RDH (prRDH) and RDH12 were tested for ability to catalyze the oxidation of all-trans-DROL to all-trans-DRAL. Both prRDH and RDH12 were active in converting all-trans-ROL to all-trans-RAL but much less so in converting all-trans-DROL to all-trans-DRAL (results not shown).

FIG. 12 shows the oxidation of all-trans-ROL and all-trans-DROL to the respective aldehyde. Purified ADH (Sigma) catalyzed the oxidation of all-trans-DROL to all-trans-DRAL (A) and all-trans-ROL to all-trans-RAL (B) in the presence of NAD and NADP. Control reactions using boiled enzyme were negative and show that the conversion is enzymatic. Retinoids were extracted and analyzed by normal phase HPLC. The products of the reaction were syn- and anti-all-trans-DRAL oximes (A) and syn- and anti-all-trans-RAL oximes (B). The experiment was performed in triplicate and repeated.

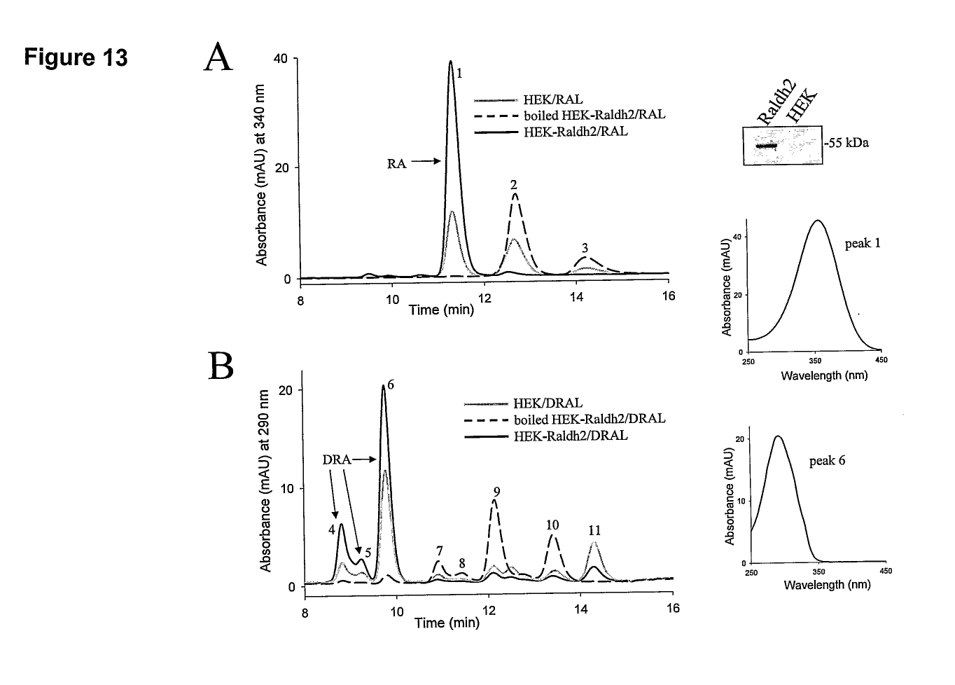

Conversion of all-trans-DRAL to DRA is mediated by RALDH enzymes. Mouse RALDH1-4 cDNAs were cloned and fused at their C terminus with a tag containing a V5 epitope and His₆ stretch. Glycosylation-deficient HEK-293S cells were transiently transfected with the tagged constructs of RALDH1, -2, -3, or -4 under the control of the CMV promoter. These cells allow the reproducible, high level expression of recombinant proteins. Reeves, et al. Proc. Natl. Acad. Sci. U.S.A. 99:13419-13424, 2002. The cell homogenate of transfected cells was supplemented with NAD, NADP, and ATP cofactors and with all-trans-RAL or all-trans-DRAL substrates. RALDH2 and -3 both efficiently converted all-trans-RAL and all-trans-DRAL into all-trans-RA and all-trans-DRA, respectively (FIGS. 13, A and B). The products all-trans-RA and all-trans-DRA were identified based on their elution time, absorbance spectra, and comparison with authentic standards (FIG. 13A, peak 1, and 13B, peak 6, and inset spectra). Other cis-DRA isomers were also produced as a result of oxidation of cis-DRAL isomers present in the synthetic mixture. The expression level of recombinant protein in transfected cell homogenate was verified by immunoblotting using anti-V5 monoclonal antibody for the presence of V5-tagged RALDH protein. This is shown for RALDH2-V5-His₆ in FIG. 13 (top right panel). Based on the intensity of the immunoreactive band, similar expression levels of RALDH1, -2, -3, or -4 were attained in transfected cells. Homogenates of RALDH1- and RALDH4-transfected cells were less efficient in oxidizing all-trans-RAL or all-trans-DRAL, possibly a consequence of the C-terminal tag affecting some isozymes more than others. Alternatively, some isozymes maybe more active than others, as seen for mouse RALDH2 (Kₘ=0.66 μm for all-trans-RAL) versus mouse RALDH1 (Kₘ=11.6 μM for all-trans-RAL) (31, 32). Untransfected cells also exhibited significant activity toward both all-trans-RAL and all-trans-DRAL (FIG. 13, gray line chromatogram), suggesting endogenous RALDH activity in HEK-293S cells.

FIG. 13 shows the oxidation of all-trans-RAL and all-trans-DRAL to all-trans-RA and all-trans-DRA, respectively. Cells were transiently transfected with vector carrying the cDNA of RALDH2 fused at its C terminus to a V5-His₆ tag. The expression of RALDH2-V5-His₆-tagged protein was confirmed by immunoblotting with anti-V5 monoclonal antibody and is shown in the top panel on the right in the lane labeled Raldh2. Cell homogenates of transfected HEK-RALDH2 cells (black solid line graph) or untransfected control cells (gray solid line graph) were incubated with all-trans-RAL (A) or all-trans-DRAL (B). Boiled control cells (black dashed line graph) were incubated with substrates under the same conditions. Retinoids were extracted and analyzed by reverse phase HPLC as described under “Methods and Materials.” The products of the reaction were identified based on their absorbance spectra and coelution with available standards. These are as follows: peak 1, all-trans-RA; peaks 2 and 3, syn- and anti-RAL oxime, respectively; peaks 4 and 5, cis-isomers of DRA; peak 6, all-trans-DRA; peaks 7-10, syn- and anti-oximes of several isomers of DRAL; peak 11, all-trans-DROL. The UV-visible absorbance spectra of peak 1 (identified as RA) and peak 6 (identified as DRA) are shown in middle and bottom panels on the right, respectively. The experiment was performed in duplicate and repeated three times. Similar results were obtained with cells transfected with RALDH3 tagged at the C terminus with V5-His₆ tag.

### EXAMPLE 17

### Oxidation of All-trans-DRA

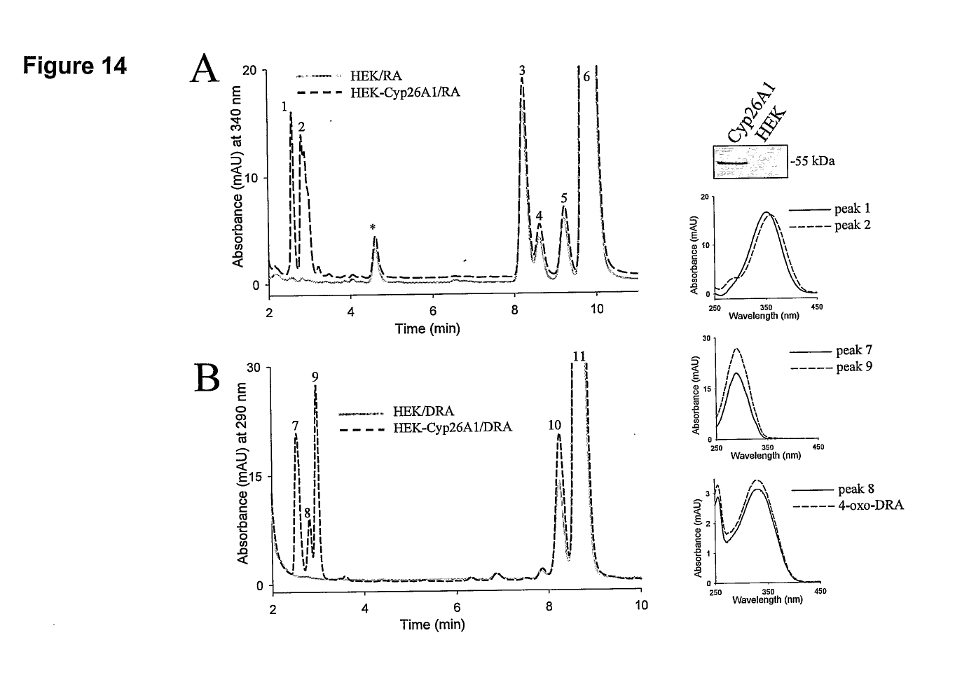

The level of RA is tightly controlled by both spatially and temporally regulated synthesis and degradation. RA catabolism is carried out by cytochrome P450 enzymes CYP26A1, -B1, and -C1. It is important to determine whether DRA could also be catabolized in a similar manner. HEK-293S cells were transfected with expression constructs of CYP26A1, -B1, and -C1 fused at their C termini with a V5 epitope and His6 stretch. Transfected and untransfected cells were incubated with all-trans-RA or all-trans-DRA substrate in culture because CYP26A1, -B1, and -C1 activity was adversely affected by homogenization of cells. Oxidized metabolites of all-trans-RA and all-trans-DRA were present in CYP26A1-transfected cells but not in untransfected cells (FIGS. 14, A and B). These metabolites, which could include all-trans-4-oxo-(D)RA, all-trains-4-hydroxy-(D)RA, all-trans-5,8-epoxy-(D)RA, and all-trans-18-hydroxy-(D)RA, were identified as polar compounds eluting shortly after the injection spike (FIGS. 14, A and B, peaks 1 and 2 and peaks 7-9, and inset spectra). One of the oxidized all-trans-DRA compounds was identified as all-trans-4-oxo-DRA because it matched the elution profile and absorbance spectrum of a synthetic standard (FIG. 14, lower right, inset panel). The level of tagged enzyme expressed in transfected cells was assayed by SDS-PAGE analysis of transfected cell lysates, followed by immunoblotting using an anti-V5-monoclonal antibody (FIG. 14, top right panel). The level of expression of CYP26A1, -B1, and -C1 in transfected cells was similar, and all three enzymes efficiently carried out the oxidation of all-trans-RA and all-trans-DRA to polar metabolites.

FIG. 14 shows the oxidation of all-trans-RA and all-trans-DRA. The metabolism of RA and DRA was examined in untransfected cells or cells transfected with CYP26A1. The expression of CYP26A1-V5-His6-tagged protein in transfected cells was examined by SDS-PAGE and immunoblotting with anti-V5 monoclonal antibody and is shown in the top panel on the right in the lane labeled Cyp26A1. Transfected HEK-CYP26A1 cells (black dashed line graph) or untransfected control cells (gray solid line graph) were incubated with RA (A) or DRA (B). Retinoids were extracted and analyzed by reverse phase HPLC as described under “Materials and Methods.” The spectra of oxidized RA metabolites, peaks 1 and 2 (right top inset panel), resemble those of all-trans-4-hydroxy-RA and all-trans-4-oxo-RA, respectively. Peaks 3-5 are cis-isomers of RA; peak 6 is all-trans-RA. Peaks 7 and 9 represent oxidized DRA metabolites and have a max λ=290 nm shown in the middle inset panel on the right. Peak 8 corresponds to all-trans-4-oxo-DRA based on its absorbance spectra and elution time (absorbance spectra shown in lower inset panel on the right). Peaks 10 and 11 represent cis- and all-trans-DRA, respectively. The experiment was performed in duplicate and repeated three times. Similar results were obtained with cells transfected with CYP26B1 and -C1.

### EXAMPLE 18

### Conversion of All-trans-DROL to All-trans-DRA in RPE

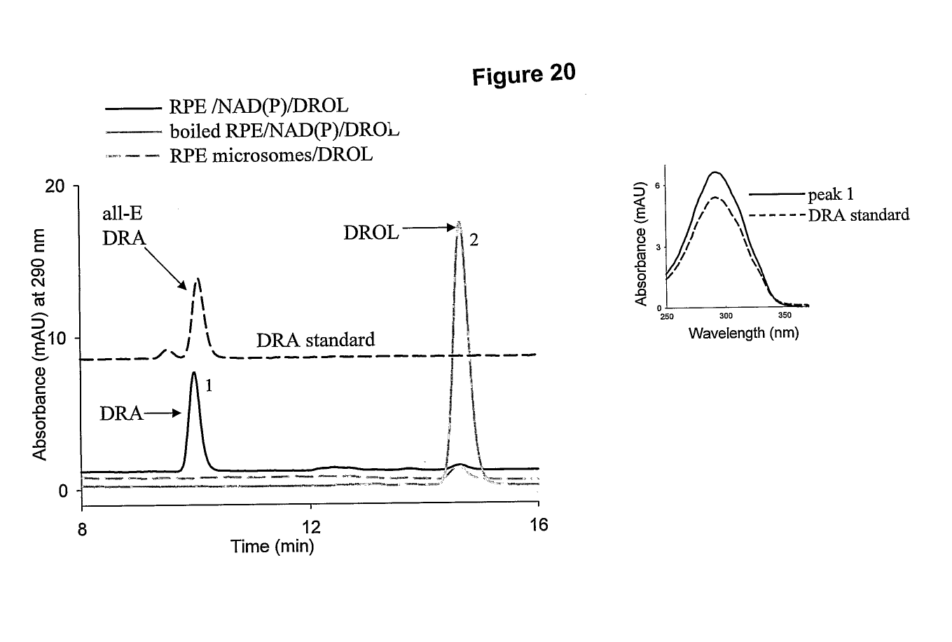

Retinoid metabolism occurs in many embryonic and adult tissues. Thus, it is important to determine whether the entire pathway of synthesis of all-trans-DRA can be reconstituted with tissue extracts. All-trans-DROL (FIG. 20, peak 2) was efficiently converted to all-trans-DRA (FIG. 20, peak 1) by microsomes prepared from RPE cells in the presence of dinucleotide cofactors NAD and NADP. All-trans-DRA was identified based on its elution profile and absorbance spectrum in comparison with synthetic all-trans-DRA (FIG. 20 and inset spectra). RPE microsomes also catalyzed the conversion of all-trans-ROL into all-trans-RA (results not shown), which indicates that adult RPE could be an active all-trans-RA, all-trans-DRA synthesis site. The main ROL oxidizing activity in the RPE is catalyzed by SDR family enzymes. The efficient conversion of all-trans-DROL to all-trans-DRA in the RPE supports the existence of SDR enzymes that can convert all-trans-DROL into all-trans-DRAL. Further studies are required to examine the substrate specificity of the known SDR enzymes from the RPE with respect to all-trans-DROL.

FIG. 20 shows conversion of all-trans-DROL into all-trans-DRA by RPE microsomes. RPE microsomes were incubated with all-trans-DROL in the presence or absence of dinucleotide cofactors NAD and NADP. As a control we incubated boiled RPE microsomes with all-trans-DROL in the presence of cofactors NAD and NADP (gray solid line). Proteins were precipitated using an equal volume of CH3CN and high-speed centrifugation. The supernatant was injected into a reverse-phase HPLC system and the elution of all-trans-DROL metabolites was monitored at 290 nm. Peak 2 (DROL) was converted to peak 1, identified as all-trans-DRA based on its coelution with an authentic standard (black dashed-line chromatogram). (B) The spectra of peak 1 and the all-trans-DRA standard are shown in inset panel. The experiment was performed in triplicate and repeated.

Based on the known all-trans-ROL oxidation pathway and results presented here, we propose that following saturation of all-trans-ROL to all-trans-DROL, all-trans-DROL is oxidized to all-trans-DRA and later to all-trans-4-oxo-DRA and possibly other oxidized metabolites of all-trans-DRA. We showed that the same enzymes involved in the oxidation of ROL to RA are also involved in the oxidation of DROL to DRA as depicted in FIG. 24. All-trans-DROL and other more oxidized metabolites occur naturally and represent a novel and potentially important pathway in the metabolism of vitamin A. This hypothesis is supported by the unequivocal identification of all-trans-DROL and all-trans-DRA in Lrat−/− mice gavaged with all-trans-ROL palmitate.

### EXAMPLE 19

### Characterization of the Transactivation Activity of All-trans-DRA

All-trans-RA binding to RAR and 9-cis-RA binding to RAR or RXR can control the expression of genes containing RA-response element (RARE) sequences within their promoter region. RARE elements are composed of direct repeats (DR) of the canonical sequence PuG(G/T)TCA separated by one to five nucleotides. Activated RAR/RXR heterodimers can associate with RARE composed of DR separated by five nucleotides (DR5), which are found in the promoter region of many genes including the RAR gene. Sucov, et al. Proc. Natl. Acad. Sci. U.S.A. 87:5392-5396, 1990.

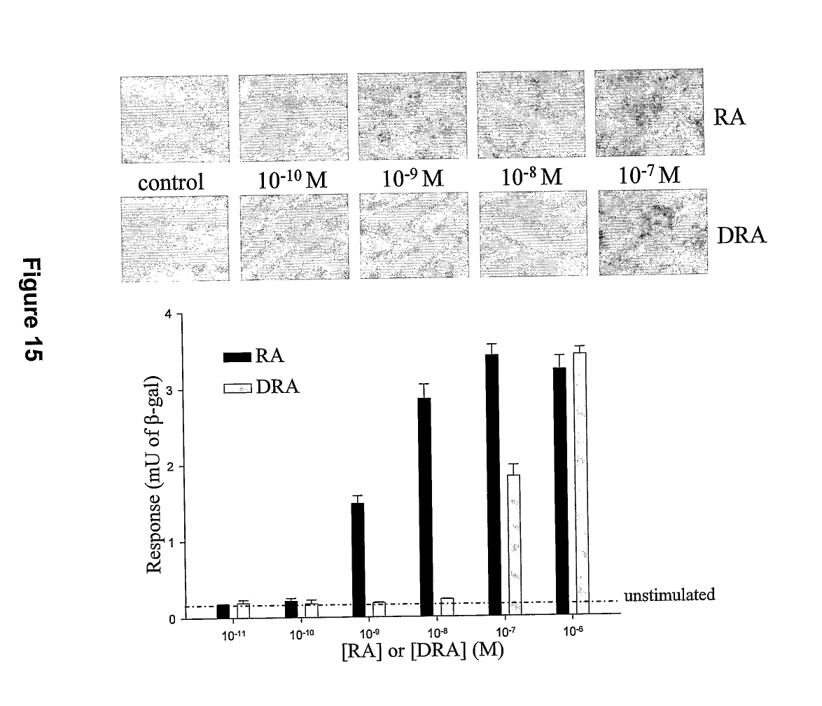

We studied whether DRA could also control gene expression through RAR activation by using a DR5 RARE-reporter cell line. The F9 teratocarcinoma cell line expresses endogenous RAR and RXR and is exquisitely sensitive to the effects of RA. This cell line has been transfected with lacZ under the control of a minimal promoter and upstream DR5 elements. Wagner, et al. Development (Camb.) 116:55-66, 1992. F9-RARE-lacZ cells were treated with different doses of all-trans-RA or all-trans-DRA for 24 h, after which the cells were harvested, and the β-galactosidase activity was evaluated by X-gal staining (FIG. 15, top panels). All-trans-DRA transactivation of DR5-induced β-galactosidase expression was observed at higher concentrations than the equivalent effect produced by RA. All-trans-RA and all-trans-DRA induction activity was quantified by using the soluble substrate o-nitrophenyl-D-galactopyranoside. The colorless substrate was cleaved by β-galactosidase to yellow colored o-nitrophenol, whose absorbance was measured at 420 nm using a spectrophotometer (FIG. 15). All-trans-DRA induction of DR5 elements is much less efficient than that of all-trans-RA. Induction of DR5 reporter cells with 10−9 M all-trans-RA had a magnitude similar to the one obtained with 10−7 M all-trans-DRA. The response measured in the linear part of the dose-response curve showed that all-trans-DRA is about 100-fold less effective than all-trans-RA in activating DR5-response elements.

FIG. 15 shows the response of F9-RARE-lacZ reporter cell line to RA and DRA. F9-RARE-lacZ cells express endogenous RAR and RXR and were transfected with a construct of lacZ under the control of a minimal promoter and upstream DR5 elements. Wagner, et al. Development (Camb.) 116:55-66, 1992. F9-RARE-lacZ cells were treated with different doses of all-trans-RA or all-trans-DRA for 24 h. The RARE-driven lacZ gene produces β-galactosidase, which hydrolyzes X-gal to an insoluble blue product, which was visualized in responder cells by light microscopy (top panels). Alternatively, the response of the cell population was quantified by measuring the β-galactosidase activity using the substrate o-nitrophenyl β-D-galactopyranoside. The colorless substrate was hydrolyzed by β-galactosidase to soluble, yellow-colored o-nitrophenol, whose absorbance was measured at 420 nm using a spectrophotometer (bottom, bar graph). The background β-galactosidase activity in unstimulated cells is indicated by dashed line. The experiment was repeated twice with similar results.

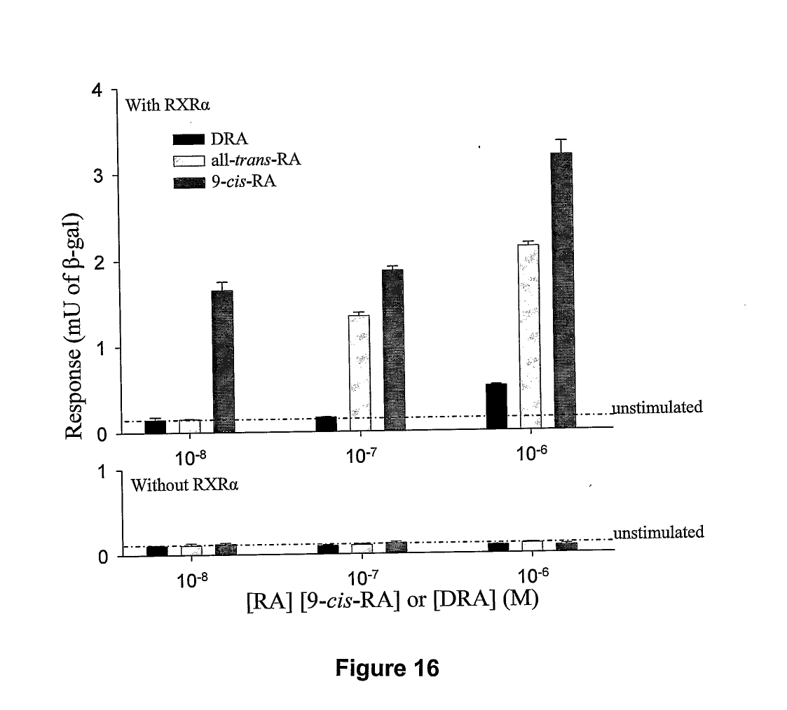

RXR homodimers can be activated by 9-cis-RA, phytanic acid, docosahexanoic acid, and other unsaturated fatty acids. Heyman, et al. Cell 68:397-406, 1992; Lemotte, et al. Eur. J. Biochem. 236:328-333, 1996; de Urquiza, et al. Science 290:2140-2144, 2000; Goldstein, et al. Arch. Biochem. Biophys. 420-185-193, 2003. RXR homodimers can bind DR1 elements of hexameric motifs separated by a single base pair as found in the CRBP II promoter. Mangelsdorf, et al. Cell 66:555-561, 1991. We studied activation of RXR based on a DR1-reporter cell assay using HEK-293S cells with a construct of lacZ under the control of a minimal promoter and five consecutive upstream DR1 elements, which we termed pRXRE-BLUE. Because HEK-293S cells express little endogenous RXR, there is no induction of DR1 elements by 9-cis-RA in the absence of exogenous RXR (FIG. 16, bottom graph). Thus, we cloned mouse RXR-α and expressed it under the control of the CMV promoter in HEK-293S cells, which we cotransfected with pRXRE-BLUE. In our assay 9-cis-RA activates RXR-mediated transcription, whereas all-trans-DRA was a very weak RXR activator (FIG. 16, top graph). Even though all-trans-RA does not bind RXR, we found that addition of all-trans-RA also resulted in robust induction of RXR homodimers in comparison with all-trans-DRA. This result could be a consequence of all-trans-RA isomerization to 9-cis-RA during the overnight incubation.

FIG. 16 shows the activation of DR1 elements by all-trans-DRA, all-trans-RA, and 9-cis-RA. HEK-293S cells were transfected with a construct of lacZ under the control of a minimal promoter and five consecutive upstream DR1 elements. Top, HEK-293S cells were cotransfected with both DR1-reporter construct and mouse RXR-α under the control of the CMV promoter. The cells were then treated with the indicated levels of all-trans-RA, 9-cis-RA, or all-trans-DRA for 48 h. The cells were harvested, and β-galactosidase activity was assayed as described under “Materials and Methods.” Bottom, DR1-reporter transfected cells were treated with different doses of all-trans-RA, 9-cis-RA, or all-trans-DRA in the absence of RXR for 48 h. The background β-galactosidase activity in unstimulated cells is indicated by the dashed line in both upper and lower graphs. The cells were harvested, and β-galactosidase activity was assayed as described under “Materials and Methods.” The experiment was repeated twice with similar results.

### EXAMPLE 20

Identification of All-trans-DRA and other 13,14-Dihydroretinoid Metabolites

In this study we identify all-trans-DRA and other 13,14-dihydroretinoid metabolites in the tissues of Lrat−/− mice supplemented with ROL palmitate, and we demonstrate that all-trans-DRA can control gene expression in reporter cell assays. All-trans-DRA stimulated expression of a DR5-RARE reporter gene by activating RAR/RXR heterodimers in F9-RARE-lacZ cells. All-trans-DRA did not activate RXR homodimers in HEK-293S cells cotransfected with a DR1-lacZ reporter construct and mouse RXR-α. In combination with a previous report on the identification of all-trans-DROL as the product of RetSat, this study characterized the enzymatic pathway responsible for the formation of all-trans-DRA from all-trans-ROL. Moise, et al. J. Biol. Chem. 279:50230-50242, 2004. Saturation of the C13-14 bond of all-trans-ROL by RetSat produces all-trans-DROL, which is oxidized to the corresponding retinaldehyde, all-trans-DRAL, by ADH-1 and possibly by SDR family RDHs present in the RPE. All-trans-DRAL is oxidized to all-trans-DRA by RALDH1-4. All-trans-DRA can be oxidized to all-trans-4-oxo-DRA in mice gavaged with all-trans-DROL and in vitro by cytochrome P450 enzymes CYP26A1, -B1, and -C1, suggesting a possible pathway for its degradation (FIG. 24). All the substrates and products of reactions and metabolites isolated from mouse tissues were identified by comparing their UV-visible absorbance spectra and chromatographic profile with authentic synthetic standards characterized by NMR and mass spectrometry. Contrary to a previous report indicating the conversion of 9-cis-RA to 9-cis-DRA, we found no evidence of in vivo conversion of all-trans-RA into all-trans-DRA. Shirley, et al. Drug Metab. Dispos. 24:293-302, 1996. Thus, all-trans-DRA can only be derived from oxidation of all-trans-DROL, and RetSat is the sole known enzyme responsible for catalyzing the key step in all-trans-DRA formation. These findings indicate that saturation of all-trans-ROL by RetSat is an active and possibly important step in the metabolism of retinoids in vivo.

Synthesis and Degradation of All-trans-DRA. Many of the ADH and SDR families and some RALDHs are expressed in the retina and RPE. Mic, et al. Mech. Dev. 97:227-230, 2000; Haeseleer, et al. Methods Enzymol 316:372-383, 2000; Wagner, et al. Dev. Biol. 222:460-470, 2000; Mic, et al. Dev. Dyn. 231:270-277, 2004; Fischer, et al. J. Neurocytol. 28:597-609, 1999; Mey, et al. Res. Dev. Brain Res. 127:135-148, 2001. We demonstrate in the current study that a pathway of conversion of all-trans-DROL into all-trans-DRA exists and is efficient in RPE microsomes (FIG. 20). This implies that all-trans-DRA synthesis can occur in the same tissues where all-trans-RA synthesis occurs and that all-trans-DRA could have a concentration gradient in different tissues. This gradient will be determined by the availability of synthetic and catabolic enzymes as well as the availability of primary substrate, i.e. all-trans-DROL.

RA bioavailability is tightly regulated by the balance between its biosynthesis and catabolism. Niederreither, et al. Nat. Genet. 31:84-88, 2002. The cytochrome P450-type enzymes, which include ubiquitously expressed CYP26A1, -B1, and -C1, oxidize RA to 4-OH—RA, 4-oxo-RA, 18-OH—RA, and 5,8-epoxy-RA. Fujii, et al. EMBO J. 16:4163-4173, 1997; White, et al. J. Biol. Chem. 272:18538-18541, 1997; Taimi, et al. J. Biol. Chem. 279:77-85, 2004; MacLean, et al. Mech. Dev. 107:195-201, 2001. Thus, CYP26 enzymes are involved in limiting spatial and temporal levels of RA, and in concert with ADH, SDR, and RALDH they guard a desirable level of RA, protecting against fluctuations in the nutritional levels of ROL. As shown here, CYP26A1, -B1, and -C1 enzymes also metabolize all-trans-DRA. This could also contribute to a temporal and spatial gradient of DRA in vivo.

Identification of Chain-shortened ROL Metabolites. In this study we report the identification of an ROL metabolite that contains an alcohol functional group and is saturated at the C13-14 bond and chain-shortened at C-15. Chain-shortened ROL metabolites have been described in early studies that followed the fate of radioactive ¹⁴C-labeled RA or all-trans-ROL. Wolf, et al. J. Am. Chem. Soc. 79:1208-1212, 1957; Roberts, et al. J. Lipid Res. 9:501-508, 1968. Yagishita, et al. Nature 203:411-412, 1964. One possible pathway for their synthesis could be through α-oxidation of all-trans-DRA as suggested previously by others. Shirley, et al. Drug Metab. Dispos. 24:293-302, 1996. The C19-ROL metabolite could be the product of a reduced C19-aldehyde intermediate produced during the α-oxidation of all-trans-DRA (equivalent to the pristanal intermediate of the phytanic acid degradation pathway). Only low amounts of C19-ROL were observed in Lrat−/− mice supplemented with all-trans-DROL compared with the levels obtained in mice gavaged with all-trans-ROL palmitate. This discrepancy might be accounted for by the fact that endogenous all-trans-DROL has access to a different repertoire of enzymes than does all-trans-DROL administered by gavage. The definite pathway of synthesis of the C19-ROL could be established by using knock-out animal models deficient in specific enzymes of this pathway.

Potential Role of 13,14-Dihydroretinoids in Vertebrate Physiology. Based on experiments using mice deficient in specific enzymes involved in retinoid metabolism, it was shown that ADH1 and RALDH1 are involved in a protection mechanism in response to pharmacological doses of ROL. Adh1−/− and Raldh1−/− mice were much more sensitive to ROL-induced toxicity than their wild type counterparts. Molotkov, et al. J. Biol. Chem. 278:36085-36090, 2003; Molotkov, et al. Biochem. J. 383:295-302, 2004. It was proposed that conversion of ROL to RA protects against excess levels of dietary ROL. This idea is counterintuitive considering the well known toxic effects of RA. Here we show that ADH1 and RALDH1 are also involved in DROL oxidation to DRA and that all-trans-DRA is a much weaker activator of RAR- or RXR-mediated transcription compared with all-trans or 9-cis-RA. Thus, it is possible that saturation of the C13-14 bond of all-trans-ROL could be the first step in a degradation pathway, which provides protection against pharmacological doses of all-trans-ROL and circumvents the formation of RA. Our findings show that the combined amounts of hepatic DROL and DROL metabolites amount to less than one-third of the amount of hepatic all-trans-RA at 3 h post-gavage with 10° IU ROL palmitate/kg body weight. This would suggest that saturation by RetSat is a rate-limiting reaction in the metabolic pathway.

Another possibility is that RetSat activity leads to production of novel bioactive 13,14-dihydroretinoids. We identify all-trans-DRA as an activator of RAR/RXR heterodimer-mediated transcription. The tissue concentration and transactivation profile of all-trans-DRA are both lower than those of all-trans-RA. It is possible that all-trans-DRA and other DROL metabolites could have important transactivation activity in certain physiological circumstances. The local concentration of 13,14-dihydroretinoid ligand might reach higher levels as a result of being trapped by receptors or binding proteins. Given that the local concentration and binding affinity are sufficient, all-trans-DRA could be an important endogenous ligand for RAR or possibly for other nuclear receptors. The finding that the same enzymes that were thought to act specifically in the formation of RA are also responsible for the formation of DRA has to be considered in attempts to rescue with RA the phenotype of knockout animal models deficient in these enzymes. In one such example, Raldh2−/− mouse embryos cannot be completely rescued by maternal RA supplementation and die prenatally. Niederreither, et al. Development (Camb.) 130:2525-2534, 2003. It is interesting to speculate if other retinoid metabolites, including 13,14-dihydroretinoids, in addition to RA may be necessary for a complete rescue of Raldh2−/− embryos. The identification of the all-trans-DRA metabolic pathway is the first step in this process, and more studies are necessary to establish the physiological role of DRA and other DROL metabolites in controlling gene expression.

In summary, we describe a new metabolic pathway for vitamin A that leads to a new class of endogenous bioactive retinoids. We demonstrate that all-trans-ROL saturation to all-trans-DROL followed by oxidation to all-trans-DRA occurs in vivo. All-trans-DRA can activate transcription of reporter genes by binding RAR but does not bind RXR. The oxidative pathway of all-trans-DROL employs the same enzymes as that of all-trans-ROL. We expect that these previously unknown metabolites will help us better understand the vital functions of retinoids in vertebrate physiology.

The abbreviations used are: ROL, retinol; ROL palmitate, retinyl palmitate; ADH, medium-chain alcohol dehydrogenases; 9-cis-DRA, 9-cis-13,14-dihydroretinoic acid; C19-ROL, (3E,5E,7E)-2,6-dimethyl-8-(2,6,6-trimethylcyclohex-1-enyl)octa-3,5,7-trien-1-ol; DRAL, 13,14-dihydroretinaldehyde; DROL, 13,14-dihydroretinol; LRAT, lecithin:retinol acyltransferase; RA, retinoic acid; RAL, retinaldehyde; RALDH, RAL dehydrogenase; RAR, retinoic acid receptor; RetSat, all-trans-ROL:all-trans-DROL saturase; RXR, retinoid X receptor; SDR, short-chain dehydrogenase/reductase; RARE, RAR element; RXRE, RXR element; cytomegalovirus; HPLC, high pressure liquid chromatography; MGC, Mammalian Gene Collection; DR, direct repeats; X-gal, 5-bromo-4-chloro-3-indolyl-D-galactopyranoside.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method of producing all-trans-(13,14)-dihydroretinol, comprising expressing a heterologous nucleic acid which hybridizes under stringent conditions comprising hybridization in aqueous solution containing 4-6×SSC at 65-68° C., or 42° C. in 50% formamide, to a polynucleotide that codes for human RetSat (GenBank Accession Number gi46329587), mouse RetSat (GenBank Accession Number AY704159) or monkey (macaque) RetSat (GenBank Accession Number AY707524), or a functionally active fragment thereof, in a host cell.

2. The method of claim 1, wherein the host cell is a mammalian host cell.

3. An isolated polypeptide comprising the contiguous sequence of human, mouse or monkey all-trans-retinol: all-trans-13,14-dihydroretinol saturase, or a functionally active fragment thereof.

4. The isolated polypeptide of claim 3 further comprising the contiguous sequence of human all-trans-retinol: all-trans-13,14-dihydroretinol saturase (GenBank Accession Number gi46329587).

5. An isolated polynucleotide comprising the contiguous sequence of human, mouse or monkey all-trans-retinol: all-trans-13,14-dihydroretinol saturase, or a functionally active fragment thereof.

6. An expression construct comprising the following operably linked elements: a transcriptional promoter; a RETSAT polynucleotide which hybridizes under stringent conditions comprising hybridization in aqueous solution containing 4-6×SSC at 65-68°, or 42° C. in 50% formamide, to a polynucleotide encoding human RetSat (GenBank Accession Number gi46329587), mouse RetSat (GenBank Accession Number AY704159) and monkey (macaque) RetSat (GenBank Accession Number AY707524) or the full length complement of the polynucleotide, wherein the Retsat polypeptide comprises the contiguous amino acid sequence of the human, mouse or monkey polypeptide or a functionally active fragment thereof; and a transcriptional terminator.

7. The expression construct of claim 6, wherein the transcriptional promoter is a heterologous promoter.

8. A cultured prokaryotic or eukaryotic cell transformed or transfected with the expression construct of claim 6.

9. The eukaryotic cell of claim 8, wherein the eukaryotic cell is a mammalian cell.

10. A vector comprising the expression construct of claim 6.

11. An isolated host cell comprising the vector of claim 10.

12. A method for producing a Retsat polypeptide, which comprises: growing cells transformed or transfected with the vector of claim 10; and isolating the Retsat polypeptide from the cells.

13. The method of claim 12, wherein the cells are bacterial cells or mammalian cells.

14. An antibody that binds to human Retsat polypeptide.

15. The antibody of claim 14, which is a monoclonal antibody, a polyclonal antibody, a single chain antibody, a heavy chain antibody, an F(ab′)₂, F(ab′), or Fv fragment.

16. A method of identifying agonists or antagonists of a eukaryotic Retsat polypeptide comprising: administering a candidate compound to a first cell that expresses a Retsat polypeptide, and determining whether the candidate compound produces a physiological change by the first cell.

17. A pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

18. The pharmaceutical composition of claim 17, formulated for topical administration, oral administration, intravenous administration, intraocular injection or perioccular injection.

19. The pharmaceutical composition of claim 17, wherein the all-trans 13,14-dihydroretinoid derivative is a retinyl ester.

20. A method or treating a neoplastic disease in a mammalian subject comprising administering to the mammalian subject a pharmaceutical composition comprising a nucleic acid construct expressing the polynucleotide of claim 3.

21. A method for treating for treating retinal disease or blindness in a mammalian subject comprising administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

22. A method for treating a retinal disease state or blindness in a mammalian subject comprising administering to the mammalian subject a compound that activates all-trans-retinol: all-trans-13,14-dihydroretinol saturase activity in the mammalian subject.

23. A method for treating for treating autoimmune disease in a mammalian subject comprising administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

24. A method for treating autoimmune disease in a mammalian subject comprising administering to the mammalian subject a compound that activates all-trans-retinol: all-trans-13,14-dihydroretinol saturase activity in the mammalian subject.

25. A method for treating for treating a skin condition or disorder in a mammalian subject comprising administering to the mammalian subject a pharmaceutical composition comprising all-trans-13,14-dihydroretinol, all-trans-13,14-dihydroretinoic acid and/or all-trans-13,14-dihydroretinoid derivative, and a pharmaceutically acceptable carrier.

26. A method for treating a skin condition or disorder in a mammalian subject comprising administering to the mammalian subject a compound that activates all-trans-retinol: all-trans-13,14-dihydroretinol saturase activity in the mammalian subject.

27. A method for treating a neoplastic disease state in a mammalian subject comprising administering to the mammalian subject a compound that inhibits all-trans-retinol: all-trans-13,14-dihydroretinol saturase activity in a neoplastic cell.


SEQUENCE LISTING (41 sequences)

SEQ ID NO: 1 - DNA, 25 bp, Artificial Sequence
  Oligonucleotide primer
atgtggatcactgctctgctgctgg

SEQ ID NO: 2 - DNA, 28 bp, Artificial Sequence
  Oligonucleotide primer
tctggctcttctctgaacggactacatc

SEQ ID NO: 3 - DNA, 23 bp, Artificial Sequence
  Oligonucleotide primer
cagtcggagctgtcccatttacc

SEQ ID NO: 4 - DNA, 25 bp, Artificial Sequence
  Oligonucleotide primer
aaattcctctgactcctccctgatg

SEQ ID NO: 5 - DNA, 20 bp, Artificial Sequence
  Oligonucleotide primer
ctttccagggagcccaaaat

SEQ ID NO: 6 - DNA, 27 bp, Artificial Sequence
  Oligonucleotide primer
acatctagatatcatgctagtgtcctt

SEQ ID NO: 7 - DNA, 39 bp, Artificial Sequence
  Oligonucleotide primer
cctctagagccaccatgtggatcactgctctgctgctgg

SEQ ID NO: 8 - DNA, 39 bp, Artificial Sequence
  Oligonucleotide primer
actagtctacatcttcttcttttgtgccttgacctttga

SEQ ID NO: 9 - DNA, 44 bp, Artificial Sequence
  Oligonucleotide primer
tctagaaggaggacagcaatggtagatgtagacaaaagagtgga

SEQ ID NO: 10 - DNA, 27 bp, Artificial Sequence
  Oligonucleotide primer
acatctagatatcatgctagtgtcctt

SEQ ID NO: 11 - DNA, 30 bp, Artificial Sequence
  Oligonucleotide primer
gccaccatgaagaacccaatgctggaagct

SEQ ID NO: 12 - DNA, 24 bp, Artificial Sequence
  Oligonucleotide primer
acatacacgttgacctgtggactg

SEQ ID NO: 13 - DNA, 26 bp, Artificial Sequence
  Oligonucleotide primer
caccgcaatgtcttcgcctgcacaac

SEQ ID NO: 14 - DNA, 23 bp, Artificial Sequence
  Oligonucleotide primer
gctggcttctttaggagttcttc

SEQ ID NO: 15 - DNA, 25 bp, Artificial Sequence
  Oligonucleotide primer
cacctgcgaaccagttatggctacc

SEQ ID NO: 16 - DNA, 21 bp, Artificial Sequence
  Oligonucleotide primer
gcctgttcctcaggggttctt

SEQ ID NO: 17 - DNA, 22 bp, Artificial Sequence
  Oligonucleotide primer
caccaagcggctgccaacatgc

SEQ ID NO: 18 - DNA, 21 bp, Artificial Sequence
  Oligonucleotide primer
gctgagaccagagtgaggcta

SEQ ID NO: 19 - DNA, 24 bp, Artificial Sequence
  Oligonucleotide primer
cacccattctcgccatgatttcct

SEQ ID NO: 20 - DNA, 20 bp, Artificial Sequence
  Oligonucleotide primer
ccaaggctagagaagcaacg

SEQ ID NO: 21 - DNA, 26 bp, Artificial Sequence
  Oligonucleotide primer
caccatggcctcgctgcagctcctgc

SEQ ID NO: 22 - DNA, 23 bp, Artificial Sequence
  Oligonucleotide primer
ggagttcttctgggggatcttca

SEQ ID NO: 23 - DNA, 25 bp, Artificial Sequence
  Oligonucleotide primer
cacctgtacacagagggcactttcc

SEQ ID NO: 24 - DNA, 34 bp, Artificial Sequence
  Oligonucleotide primer
gtatttaatggtaatggtttttatttcagtaaag

SEQ ID NO: 25 - DNA, 24 bp, Artificial Sequence
  Oligonucleotide primer
caccatggggctcccggcgctgct

SEQ ID NO: 26 - DNA, 25 bp, Artificial Sequence
  Oligonucleotide primer
gatatctccctggaagtgggtaaat

SEQ ID NO: 27 - protein, 14 aa, Artificial Sequence
  Synthetic construct
GKPIPNPLLGLDST

SEQ ID NO: 28 - DNA, 20 bp, Artificial Sequence
  Oligonucleotide primer
gggcatgagttagtcgcaga

SEQ ID NO: 29 - DNA, 19 bp, Artificial Sequence
  Oligonucleotide primer
agctgagcagctgtgtcca

SEQ ID NO: 30 - DNA, 24 bp, Artificial Sequence
  Oligonucleotide primer
caccatggacaccaaacatttcct

SEQ ID NO: 31 - DNA, 24 bp, Artificial Sequence
  Oligonucleotide primer
ctcaaccctatctcggtctattct

SEQ ID NO: 32 - DNA, 23 bp, Artificial Sequence
  Oligonucleotide primer
atgccagcttcattatataccca

SEQ ID NO: 33 - protein, 610 aa, Homo sapiens [sequence omitted]

SEQ ID NO: 34 - protein, 610 aa, Macaque
MWLPLVLFLAVLLLAVVCKVYLGLFSGKSPNPFSEDVKRPPAPLVTDKEARKKVLKQAFSASRVPEKLDVVVIGSGFGGLAAAAILAKAGKRVLVLEQHTKAGGACHTFGENGLEFDTGIHYIGRMEEGSIGRFILDQITEGQLDWVPMSSPFDIMVLEGPNGRKEYPMYSGEKAYIQGLKEKFPQEEAIIDKYIKLVKVVSNGVAHAILLKFLPLPVIQLLDRCGLLTRFSPFLHASTQSLAEVLQQLGASSELQAVLSYIFPTYGVTPRHSAFSMHALLVNHYLKGAFYPRGGSSEIAFHTIPVIQRAGGAVLTRATVQSVLLDSAGKACGVSVKKGHELVNIYCPVVVSNAGLFNTYEHLLPGNARCLPGVKQQLGMVRPGLGMMSVFICLQGTKEDLHLPSTNYYVYHDTDMDQAMERYVSMPREKAAEHIPLLFIAFPSAKDPTWEDRFPGRSSMIMLIPSAYEWFEEWQAELKGKRGSDYETYKNSFVEASMSVAMKLFPQLEGKVESVTAGSPLTNQFYLAAPRGACYGADHDLGRLHPRVMASLRAQSPIPNLYLTGQDIFTCGLVGALQGALLCSSAILKRNLYSDLKDLGSRIQAQKKKN

SEQ ID NO: 35 - protein, 609 aa, Mus musculus
MWITALLLAVLLLVILHRVYVGLYAASSPNPFAEDVKRPPEPLVTDKEARKKVLKQAFSVSRVPEKLDAVVIGSGIGGLASAAVLAKAGKRVLVLEQHTKAGGCCHTFGENGLEFDTGIHYIGRMREGNIGRFILDQITEGQLDWAPMASPFDLMILEGPNGRKEFPMYSGRKEYIQGLKKKFPKEEAVIDKYMELVKVVARGVSHAVLLKFLPLPLTQLLSKFGLLTRFSPFCRASTQSLAEVLQQLGASRELQAVLSYIFPTYGVTPSHTAFSLHALLVDHYIQGAYYPRGGSSEIAFHTIPLIQRAGGAVLTRATVQSVLLDSAGRACGVSVKKGQELVNIYCPVVISNAGMFNTYQHLLPETVRHLPDVKKQLAMVRPGLSMLSIFICLKGTKEDLKLQSTNYYVYFDTDMDKAMERYVSMPKEKAPEHIPLLFIAFPSSKDPTWEERFPDRSTMTALVPMAFEWFEEWQEEPKGKRGVDYETLKNAFVEASMSVIMKLFPQLEGKVESVTGGSPLTNQYYLAAPRGATYGADHDLARLHPHAMASIRAQTPIPNLYLTGQDIFTCGLMGALQGALLCSSAILKRNLYSDLQALGSKVKAQKKKM

SEQ ID NO: 36 - protein, 609 aa, Rattus
MWITALLLLVLLLVVVHRVYVGLFTGSSPNPFAEDVKRPPEPLVTDKEARKKVLKQAFSVSRVPEKLDAVVIGSGIGGLASAAILAKAGKRVLVLEQHTKAGGCCHTFGENGLEFDTGIHYIGRMREGNIGRFILDQITEGQLDWAPMASPFDLMILEGPNGRKEFPMYSGRKEYIQGLKEKFPKEEAVIDKYMELVKVVAHGVSHAILLKFLPLPLTQLLNKFGLLTRFSPFCRASTQSLAEVLKQLGASPELQAVLSYIFPTYGVTPSHTTFSLHALLVDHYIQGAYYPRGGSSEIAFHTIPLIQRAGGAVLTRATVQSVLLDSAGRACGVSVKKGQELVNIYCPVVISNAGMFNTYQHLLPESVRYLPDVKKQLTMVKPGLSMLSIFICLKGTKEELKLQSTNYYVYFDTDMDKAMERYVSMPKEKAPEHIPLLFIAFPSSKDPTWEDRFPDRSTMTVLVPTAFEWFEEWQEEPKGKRGVDYETLKNTFLEASMSVIMKLFPQLEGKVESVTGGSPLTNQYYLAAHRGATYGADHDLARLHPHAMASLRAQTPIPNLYLTGQDIFTCGLMGALQGALLCSSAILKRNLYSDLQALGSKVRAQKKKK

SEQ ID NO: 37 - protein, 615 aa, Solanum lycopersicum
MCTLSFMYPNSLLDGTCKTVALGDSKPRYNKQRSSCFDPLIIGNCTDQQQLCGLSWGVDKAKGRRGGTVSNLKAVVDVDKRVESYGSSDVEGNESGSYDAIVIGSGIGGLVAATQLAVKGAKVLVLEKYVIPGGSSGFYERDGYKFDVGSSVMFGFSDKGNLNLITQALAAVGRKLEVIPDPTTVHFHLPNDLSVRIHREYDDFIEELVSKFPHEKEGIIKFYSECWKIFNSLNSLELKSLEEPIYLFGQFFKKPLECLTLAYYLPQNAGSIARKYIRDPGLLSFIDAECFIVSTVNALQTPMINASMVLCDRHFGGINYPVGGVGEIAKSLAKGLDDHGSQILYRANVTSIILDNGKAVGVKLSDGRKFYAKTIVSNATRWDTFGKLLKAENLPKEEENFQKAYVKAPSFLSIHMGVKADVLPPDTDCHHFVLEDDWTNLEKPYGSIFLSIPTVLDSSLAPEGHHILHIFTTSSIEDWEGLSPKDYEAKKEVVAERIISRLEKTLFPGLKSSILFKEVGTPKTHRRYLARDSGTYGPMPRGTPKGLLGMPFNTTAIDGLYCVGDSCFPGQGVIAVAFSGVMCAHRVAADLGFEKKSDVLDSALLRLLGWLRTLA

SEQ ID NO: 38 - protein, 595 aa, Arabidopsis thaliana
MDLCFQNPVKCGDRLFSALNTSTYYKLGTSNLGFNGPVLENRKKKKKLPRMVTVKSVSSSVVASTVQGTKRDGGESLYDAIVIGSGIGGLVAATQLAVKEARVLVLEKYLIPGGSSGFYERDGYTFDVGSSVMFGFSDKGNLNLITQALKAVGRKMEVIPDPTTVHFHLPNNLSVRIHREYDDFIAELTSKFPHEKEGILGFYGDCWKIFNSLNSLELKSLEEPIYLFGQFFQKPLECLTLAYYLPQNAGAIARKYIKDPQLLSFIDAECFIVSTVNALQTPMINASMVLCDRHYGGINYPVGGVGGIAKSLAEGLVDQGSEIQYKANVKSIILDHGKAVGVRLADGREFFAKTIISNATRWDTFGKLLKGEKLPKEEENFQKVYVKAPSFLSIHMGVKAEVLPPDTDCHHFVLEDDWKNLEEPYGSIFLSIPTILDSSLAPDGRHILHIFTTSSIEDWEGLPPKEYEAKKEDVAARIIQRLEKKLFPGLSSSITFKEVGTPRTHRRFLARDKGTYGPMPRGTPKGLLGMPFNTTAIDGLYCVGDSCFPGQGVIAVAFSGVMCAHRVAADIGLEKKSRVLDVGLLGLLGWLRTLA

SEQ ID NO: 39 - protein, 501 aa, Synechocystis sp.
MTVSPSYDAIVIGSGIGGLVTATQLVSKGLKVLVLERYLIPGGSAGYFEREGYRFDVGASMIFGFGDRGTTNLLTRALAAVGQALETLPDPVQIHYHLPGGLDPKVHREYEAFLQELIAKFPQEAQGIRRFYDECWQVFNCLNTMELLSLEEPRYLMRVFFQHPGACLGLVKYLPQNVGDIARRHIQDPDLLKFIDMECYCWSVVPADLTPMINAGMVFSDRHYGGINYPKGGVGQIAESLVAGLEKFGGKIRYGARVTKIIQENNQAIGVELANGEKIYGRRIVSNATRWDTFGALTGDQPLPGKEKRWRRNYQQSPSFLSLHLGVEADLLPEGTECHHILLEDWDDLEKEQGTIFVSIPTLLDPSLAPDGYHIIHTFTPSWLESWQNLSPQEYEAKKEADSGKLIDRLEAIFPGLDRALDYMEIGTPRSHRRFLGRQNGTYGPIPRRRLPGLLPMPFNRTAIPGLYCVGDSTFPGQGLNAVAFSGFACAHRLAVDLGVR

SEQ ID NO: 40 - protein, 610 aa, Homo sapiens
MWLPLVLLLAVLLLAVLCKVYLGLFSGSSPNPFSEDVKRPPAPLVTDKEARKKVLKQAFSANQVPEKLDVVVIGSGFGGLAAAAILAKAGKRVLVLEQHTKAGGCCHTFGKNGLEFDTGIHYIGRMEEGSIGRFILDQITEGQLDWAPLSSPFDIMVLEGPNGRKEYPMYSGEKAYIQGLKEKFPQEEAIIDKYIKLVKVVSSGAPHAILLKFLPLPVVQLLDRCGLLTRFSPFLQASTQSLAEVLQQLGASSELQAVLSYIFPTYGVTPNHSAFSMHALLVNHYMKGGFYPRGGSSEIAFHTIPVIQRAGGAVLTKATVQSVLLDSAGKACGVSVKKGHELVNIYCPIVVSSAGLFNTYEHLLPGNARCLPGVKQQLGTVRPGLGMTSVFICLRGTKEDLHLPSTNYYVYYDTDMDQAMERYVSMPREEAAEHIPLLFFAFPSAKDPTWEDRFPGRSTMIMLIPTAYEWFEEWQAELKGKRGSDYETFKNSFVEASMSVVLKLFPQLEGKVESVTAGSPLTNQFYLAAPRGACYGADHDLGRLHPCVMASLRAQSPIPNLYLTGQDIFTCGLVGALQGALLCSSAILKRNLYSDLKNLDSRIRAQKKKN

SEQ ID NO: 41 - protein, 609 aa, Mus musculus
MWITALLLAVLLLVILHRVYVGLYAASSPNPFAEDVKRPPEPLVTDKEARKKVLKQAFSVSRVPEKLDAVVIGSGIGGLASAAVLAKAGKRVLVLEQHTKAGGCCHTFGENGLEFDTGIHYIGRMREGNIGRFILDQITEGQLDWAPMASPFDLMILEGPNGRKEFPMYSGRKEYIQGLKKKFPKEEAVIDKYMELVKVVARGVSHAVLLKFLPLPLTQLLSKFGLLTRFSPFCRASTQSLAEVLQQLGASRELQAVLSYIFPTYGVTPSHTTFSLHALLVDHYIQGAYYPRRGSSEIAFHTIPLIQRAGGAVLTRATVQSVLLDSAGRACGVSVKKGQELVNIYCPVVISNAGMFNTYQHLLPETVRHLPDVKKQLAMVRPGLSMLSIFICLKGTKEDLKLQSTNYYVYFDTDMDKAMERYVSMPKEKAPEHIPLLFIAFPSSKDPTWEERFPDRSTMTALVPMAFEWFEEWQEEPKGKRGVDYETLKNAFVEASMSVIMKLFPQLEGKVESVTGGSPLTNQYYLAAPRGATYGADHDLARLHPHAMASIRAQTPIPNLYLTGQDIFTCGLMGALQGALLCSSAILKRNLYSDLQALGSKVKAQKKKM